(19) United States
(12) Patent Application Publication
(10) Pub. No.: US 2024/0398500 A1
(43) Pub. Date: Dec. 5, 2024

(54) TISSUE SPACERS WITH VISUAL ADDITIVE

(71) Applicant: Palette Life Sciences, Inc., Santa Barbara, CA (US)

(72) Inventors: Brent Travis GAY, Santa Barbara, CA (US); Michelle SVATOS, Santa Barbara, CA (US)

(21) Appl. No.: 18/799,913

(22) Filed: Aug. 9, 2024

Related U.S. Application Data
(63) Continuation of application No. PCT/US2023/062438, filed on Feb. 10, 2023.
(60) Provisional application No. 63/309,430, filed on Feb. 11, 2022.

(51) Int. Cl. A61B 90/00; G06T 7/00

(52) U.S. Cl. CPC A61B 90/04; G06T 7/00; A61B 2090/0815; G06T 2207/30024

(57) ABSTRACT

Provided herein are methods for decreasing the toxicity of advanced ablative cancer therapies on neighboring organs. The methods herein provide spacing between single or multiple tumor cites and immediate healthy organs while maintaining or increasing patient quality of life. Such toxicity isolation can be performed by inserting a spacer around the one or more tumor sites.

### CROSS-REFERENCE

This application is a continuation of International Application Number PCT/US2023/062438, filed Feb. 10, 2023, which claims the benefit of U.S. Provisional Application No. 63/309,430, filed Feb. 11, 2022, which are hereby incorporated by reference in their entirety.

### BACKGROUND

Generally, radiotherapy is considered as a palliative treatment option for severe or uncommon cases of melanoma. However there has recently been an increased demand for new systems and methods of melanoma management. Brachytherapy techniques also involve balloon or strut multicatheter brachytherapy, with the applicator being placed in the surgical cavity by the breast surgeon at the time of or shortly after the wide local excision.

Current therapies possess a plethora of shortcomings known in the art, including, difficulty of imaging across all imaging modalities across all cavity locations, difficulty of application, and movement of the balloon, struct, or spacer, subsequent to placement thereof.

### SUMMARY

The formulations and methods described herein comprise improved methods of radiotherapy. More specifically, the formulations and methods described herein comprise reducing a dose of radiotherapy to tissue proximate to the site of radiotherapy.

An aspect of the disclosure described herein comprises a composition comprising a viscoelastic medium; and a first visual additive, wherein said first visual additive comprises a metal, and wherein said metal has a particle diameter of greater than or equal to 80 micrometers (μm). In some embodiments, said metal is a precious metal. In some embodiments, said metal or said precious metal is chosen from the group consisting of gold (Au), iodine (I), gadolinium (Gd), iron (Fe), barium (Ba), calcium (Ca), magnesium (Mg), and combinations thereof. In some embodiments, said metal or precious metal is an isotope of gold (Au), iodine (I), gadolinium (Gd), iron (Fe), barium (Ba), calcium (Ca), magnesium (Mg), or combinations thereof. In some embodiments, said metal or precious metal is a powder. In some embodiments, said first visual additive has a radiographic density of between about 1.0 g/cm3 and 2.0 g/cm3. In some embodiments, half of said first visual additive is configured to disperse within a tissue within nine months. In some embodiments, said particle diameter of said metal or precious metal is between about 80 μm and about 120 μm. In some embodiments, said particle diameter of said metal or precious metal is about 100 μm. In some embodiments, said first visual additive further comprises one or more microbubbles. In some embodiments, the composition comprises a second visual additive. In some embodiments, said second visual additive is different than said first visual additive. In some embodiments, said second visual additive comprises one or more microbubbles. In some embodiments, said metal or precious metal is between about 0.015 wt % and about 1.5 wt % of said composition. In some embodiments, said composition is configured to be present on an imaging modality for at least 9 months. In some embodiments, said first visual additive is configured to not substantially migrate prior to or during imaging. In some embodiments, said composition is configured to be imaged within 30 min, within 90 min, within 4 hours, within 8 hours, or within 4 days of disposition.

Another aspect of the disclosure described herein comprises a method of spacing a first tissue site of a subject in need thereof from a second tissue site of said subject in need thereof, the method comprising: disposing a viscoelastic medium in a space between said first tissue site and said second tissue site, wherein said viscoelastic medium comprises one or more visualization additives. In some embodiments, the method further comprises monitoring or imaging said space between said first tissue site and said second tissue site. In some embodiments, said space between said first tissue site and said second tissue site is in a range of about 0.1 cm to about 10 cm. In some embodiments, said visualization additive is present in an amount sufficient to generate contrast when imaged by an imaging modality. In some embodiments, said viscoelastic medium comprises a volume of about 1 ml to about 50 ml. In some embodiments, said viscoelastic medium is disposed through a 10-25 gauge needle. In some embodiments, said viscoelastic medium comprises hyaluronic acid, polyethylene glycol, or dextranomers at a concentration of a range of from about 5 mg/ml to about 100 mg/ml. In some embodiments, said viscoelastic medium comprises gel particles at a size range of about 0.08 mm to about 5 mm. In some embodiments, said viscoelastic medium is disposed subcutaneous or subepidermal. In some embodiments, said first tissue site and said second tissue site are selected from a group consisting of the subject's breast, head & neck, cervix, vagina, uterus, ovaries, base of spine, skin, pancreas, liver, rectum, or lung. In some embodiments, said imaging comprises real-time imaging. In some embodiments, said imaging is performed within 30 minutes, within 90 minutes, within 4 hours, within 8 hours, or within 4 days of said disposing said viscoelastic medium. In some embodiments, said imaging comprises MRI, CT, ultrasound, or a combination thereof. In some embodiments, said imaging modality comprises MRI, CT, ultrasound, or a combination thereof. In some embodiments, said viscoelastic medium does not substantially migrate prior to and during said imaging. In some embodiments, said visualization additives comprise one or more nanoparticles. In some embodiments, said visualization additives comprise a precious metal. In some embodiments, said precious metal comprises iron or gold. In some embodiments, said viscoelastic medium is bioabsorbable. In some embodiments, said visualization additive comprises gold, iodine, gadolinium, iron, barium, calcium, magnesium, or a combination thereof. In some embodiments, the visualization additive is gold particles.

Another aspect of the disclosure described herein comprises a method of preventing or decreasing damage to a tissue proximate to a site of a radiotherapy in a subject undergoing the radiotherapy comprising injecting a bioabsorbable viscoelastic medium at the site of the radiotherapy, wherein the bioabsorbable viscoelastic medium comprises a visualization additive. In some embodiments, the injection displaces the tissue by a distance in the range of about 0.1 cm to about 10 cm. In some embodiments, the viscoelastic medium comprises gel particles. In some embodiments, the gel particles comprise hyaluronic acid or derivatives thereof. In some embodiments, the injection comprises a volume of about 1 ml to about 50 ml. In some embodiments, the injection is performed through a 10-25 gauge needle. In some embodiments, the concentration of hyaluronic acid is in the range of from about 5 mg/ml to about 100 mg/ml. In some embodiments, the gel particles have a size range of about 0.2 mm to about 5 mm. In some embodiments, the injection is subcutaneous or subepidermal. In some embodiments, migration of the viscoelastic medium is prevented or decreased. In some embodiments, the visualization additive comprises one or more nanoparticles. In some embodiments, the nanoparticles comprise a precious metal. In some embodiments, the visual additive comprises gold, iodine, gadolinium, iron, barium, calcium, magnesium, or a combination thereof. In some embodiments, the precious metal is gold. In some embodiments, a dose of the radiotherapy contacting the tissue proximate to the site of radiotherapy is reduced by about 10% to about 80%. In some embodiments, the site of the radiotherapy is selected from a group consisting of the subject's breast, head & neck, cervix, vagina, base of spine, skin, pancreas, liver, rectum or lung. The method of any one of embodiments 35 to 48, further comprising an administration of hyaluronidase at the site of radiotherapy. In some embodiments, the volume of the viscoelastic medium at the site of radiotherapy is reduced by about 1% to about 95%. In some embodiments, the administration of hyaluronidase occurs between about 0.1 hours to about 24 hours after the injection of the bioabsorbable viscoelastic medium. In some embodiments, the method further comprises imaging the site of the radiotherapy. In some embodiments, the imaging comprises continuous imaging. In some embodiments, the imaging comprises MRI, a CT scan, ultrasound, or a combination thereof.

Another aspect of the disclosure described herein comprises a method of reducing a dose of radiotherapy to a tissue proximate to a site of a radiotherapy in a subject undergoing the radiotherapy comprising an injection of a bioabsorbable viscoelastic medium at the site of the radiotherapy. In some embodiments, the injection displaces the tissue by a distance in the range of about 0.1 cm to about 10 cm. In some embodiments, the viscoelastic medium comprises gel particles. In some embodiments, the gel particles comprise hyaluronic acid or derivatives thereof. In some embodiments, the injection comprises a volume of about 1 ml to about 50 ml. In some embodiments, the injection is performed through a 10-25 gauge needle. In some embodiments, the concentration of hyaluronic acid is in the range of from about 5 mg/ml to about 100 mg/ml. In some embodiments, the gel particles have a size range of about 0.2 mm to about 5 mm. In some embodiments, the injection is subcutaneous or subepidermal. In some embodiments, migration of the viscoelastic medium is prevented or decreased. In some embodiments, the viscoelastic medium further comprises one or more nanoparticles. In some embodiments, the nanoparticles comprise a precious metal. In some embodiments, the dose of radiotherapy is reduced by about 10% to about 80%. In some embodiments, the site of the radiotherapy is selected from a group consisting of the subject's breast, head & neck, cervix, vagina, base of spine, skin, pancreas, liver, rectum, or lung. In some embodiments, the method further comprises an administration of hyaluronidase at the site of radiotherapy. In some embodiments, the volume of the viscoelastic medium at the site of radiotherapy is reduced by about 1% to about 95%. In some embodiments, the administration of hyaluronidase occurs between about 0.1 hours to about 24 hours after the injection of the bioabsorbable viscoelastic medium.

Another aspect of the disclosure described herein comprises a method of temporarily super-spacing a tissue proximate to a site of radiotherapy comprising injecting a formulation comprising cross-linked hyaluronic acid or derivatives thereof and an amount of degradable nanoparticles encapsulating hyaluronidase. In some embodiments, the amount of degradable nanoparticles encapsulating hyaluronidase is directly proportionate to a desired distance of super spacing relative to a desired time period of super spacing. In some embodiments, the method further comprises injecting a bioabsorbable viscoelastic medium in a blood vessel wherein the blood vessel is directly coupled to a tumor. In some embodiments, the viscoelastic medium comprises gel particles. In some embodiments, the gel particles comprise hyaluronic acid or derivatives thereof. In some embodiments, the injection comprises a volume of about 1 ml to about 50 ml. In some embodiments, the injection is performed through a 10-25 gauge needle. In some embodiments, the concentration of hyaluronic acid is in the range of from about 5 mg/ml to about 100 mg/ml. In some embodiments, the gel particles have a size range of about 0.2 mm to about 5 mm. In some embodiments, blood flow to the tumor is prevented or decreased. In some embodiments, migration of the viscoelastic medium is prevented or decreased. In some embodiments, the method further comprises an administration of hyaluronidase at the site of radiotherapy. In some embodiments, the administration of hyaluronidase occurs between about 0.1 hours to about 24 hours to about 30 days after the injection of the bioabsorbable viscoelastic medium. In some embodiments, the method further comprises excising the remaining tumor cells from the subject.

Another aspect of the disclosure described herein comprises a composition comprising a viscoelastic medium and a visualization additive. In some embodiments, said visualization additive is present in an amount sufficient to generate contrast when imaged by an imaging modality. In some embodiments, said viscoelastic medium comprises a volume of about 1 ml to about 50 ml. In some embodiments, said viscoelastic medium is configured to be disposed through a 10-25 gauge needle. In some embodiments, said viscoelastic medium comprises hyaluronic acid, polyethylene glycol, or dextranomers at a concentration of a range of from about 5 mg/ml to about 100 mg/ml. In some embodiments, said viscoelastic medium comprises gel particles at a size range of about 0.08 mm to about 5 mm. In some embodiments, said visualization additive configures said viscoelastic medium to be imaged, wherein said imaging comprises real-time imaging. In some embodiments, said visualization additive configures said viscoelastic medium to be imaged within 30 minutes, within 90 minutes, within 4 hours, within 8 hours, or within 4 days of said disposing said viscoelastic medium. In some embodiments, said visualization additive configures said viscoelastic medium to be imaged wherein said imaging comprises MRI, CT, ultrasound, or a combination thereof. In some embodiments, said imaging modality comprises MRI, CT, ultrasound, or a combination thereof. In some embodiments, said viscoelastic medium is configured to not substantially migrate upon displacement. In particular, in those embodiments, the viscoelastic medium is configured to not collect in organs that are not where the viscoelastic medium was injected. In some embodiments, said visualization additives comprise one or more nanoparticles. In some embodiments, said visualization additives comprise a precious metal. In some embodiments, said precious metal comprises gold. In some embodiments, the precious metal comprises gold, iodine, gadolinium, iron, barium, calcium, or a combination thereof. In some embodiments, particles of the visual additives are about 80 micrometers to about 120 micrometers. In some embodiments, said viscoelastic medium is bioabsorbable.

Also provided herein is use of a viscoelastic medium for the manufacture of a medicament. In some embodiments, more than 70% (v/v) of the particles are within the given size limits under physiological conditions, including physiological conditions associated with man. Provided herein are particles of a viscoelastic medium, which are injectable gel particles having a size, when subjected to a physiological salt solution, in the range of from 1 to 5 mm. Sub-epidermal administration of an implant comprising gel particles made of a viscoelastic medium which are considerably larger than previously used in implants made of viscoelastic media are useful in avoiding migration and/or displacement of the implant, or part thereof, from the desired site of radiative protection. Moreover, the limited displacement of the implant in combination with the considerable particle size can facilitate easy removal of the implant, if desired. In an embodiment herein, said particle size is in the range of from 1 to 2.5 mm. In some embodiments, said size is in the range of from 2.5 to 5 mm. In embodiments herein, said viscoelastic medium is selected from the group consisting of polysaccharides and derivatives thereof. In some embodiments, said viscoelastic medium is selected from stabilized glycosaminoglycans and derivatives thereof. In some embodiments, said viscoelastic medium is selected from the group consisting of stabilized hyaluronic acid, stabilized chondroitin sulfate, stabilized heparin, and derivatives thereof. In some embodiments herein, said viscoelastic medium is selected from the group consisting of cross-linked hyaluronic acid and derivatives thereof. In some embodiments, the concentration of said viscoelastic medium in said gel particles, when subjected to a physiological salt solution, is in the range of from 5 to 100 mg/ml. In some embodiments, the particles herein are injectable through a 20 gauge or larger needle by application of a pressure of 15-50 N.

Further, provided herein is a method of producing injectable gel particles of a viscoelastic medium, comprising the steps of (i) manufacturing a gel with a desired concentration of said viscoelastic medium; and (ii) mechanically disrupting said gel into gel particles having a size, when subjected to a physiological salt solution, in the range of from 1 to 5 mm.

Further, provided herein is a radiative protection implant comprising particles of a viscoelastic medium, wherein a major volume of said particles are injectable gel particles having a size, when subjected to a physiological salt solution, in the range of from 1 to 5 mm. In one embodiment of the implant, said size is in the range of from 1 to 2.5 mm. In other one embodiments of the implant, said size is in the range of from 2.5 to 5 mm.

In some embodiments, the injectable gel particles have a size of about 0.5 mm to about 5 mm. In some embodiments, the injectable gel particles have a size of about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 4.5 mm, about 0.5 mm to about 5 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 4.5 mm, about 1.5 mm to about 5 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 3 mm to about 3.5 mm, about 3 mm to about 4 mm, about 3 mm to about 4.5 mm, about 3 mm to about 5 mm, about 3.5 mm to about 4 mm, about 3.5 mm to about 4.5 mm, about 3.5 mm to about 5 mm, about 4 mm to about 4.5 mm, about 4 mm to about 5 mm, or about 4.5 mm to about 5 mm. In some embodiments, the injectable gel particles have a size of about 0.5 mm, about 1 mm, about 1.5 mm, about 2 mm, about 2.5 mm, about 3 mm, about 3.5 mm, about 4 mm, about 4.5 mm, or about 5 mm. In some embodiments, the injectable gel particles have a size of at least about 0.5 mm, about 1 mm, about 1.5 mm, about 2 mm, about 2.5 mm, about 3 mm, about 3.5 mm, about 4 mm, or about 4.5 mm. In some embodiments, the injectable gel particles have a size of at most about 1 mm, about 1.5 mm, about 2 mm, about 2.5 mm, about 3 mm, about 3.5 mm, about 4 mm, about 4.5 mm, or about 5 mm. In some embodiments, the injectable gel particles have a size of about 1 mm to about 2 mm. In some embodiments, the injectable gel particles have a size of about 1 mm to about 1.1 mm, about 1 mm to about 1.2 mm, about 1 mm to about 1.3 mm, about 1 mm to about 1.4 mm, about 1 mm to about 1.5 mm, about 1 mm to about 1.6 mm, about 1 mm to about 1.7 mm, about 1 mm to about 1.8 mm, about 1 mm to about 1.9 mm, about 1 mm to about 2 mm, about 1.1 mm to about 1.2 mm, about 1.1 mm to about 1.3 mm, about 1.1 mm to about 1.4 mm, about 1.1 mm to about 1.5 mm, about 1.1 mm to about 1.6 mm, about 1.1 mm to about 1.7 mm, about 1.1 mm to about 1.8 mm, about 1.1 mm to about 1.9 mm, about 1.1 mm to about 2 mm, about 1.2 mm to about 1.3 mm, about 1.2 mm to about 1.4 mm, about 1.2 mm to about 1.5 mm, about 1.2 mm to about 1.6 mm, about 1.2 mm to about 1.7 mm, about 1.2 mm to about 1.8 mm, about 1.2 mm to about 1.9 mm, about 1.2 mm to about 2 mm, about 1.3 mm to about 1.4 mm, about 1.3 mm to about 1.5 mm, about 1.3 mm to about 1.6 mm, about 1.3 mm to about 1.7 mm, about 1.3 mm to about 1.8 mm, about 1.3 mm to about 1.9 mm, about 1.3 mm to about 2 mm, about 1.4 mm to about 1.5 mm, about 1.4 mm to about 1.6 mm, about 1.4 mm to about 1.7 mm, about 1.4 mm to about 1.8 mm, about 1.4 mm to about 1.9 mm, about 1.4 mm to about 2 mm, about 1.5 mm to about 1.6 mm, about 1.5 mm to about 1.7 mm, about 1.5 mm to about 1.8 mm, about 1.5 mm to about 1.9 mm, about 1.5 mm to about 2 mm, about 1.6 mm to about 1.7 mm, about 1.6 mm to about 1.8 mm, about 1.6 mm to about 1.9 mm, about 1.6 mm to about 2 mm, about 1.7 mm to about 1.8 mm, about 1.7 mm to about 1.9 mm, about 1.7 mm to about 2 mm, about 1.8 mm to about 1.9 mm, about 1.8 mm to about 2 mm, or about 1.9 mm to about 2 mm. In some embodiments, the injectable gel particles have a size of about 1 mm, about 1.1 mm, about 1.2 mm, about 1.3 mm, about 1.4 mm, about 1.5 mm, about 1.6 mm, about 1.7 mm, about 1.8 mm, about 1.9 mm, or about 2 mm. In some embodiments, the injectable gel particles have a size of at least about 1 mm, about 1.1 mm, about 1.2 mm, about 1.3 mm, about 1.4 mm, about 1.5 mm, about 1.6 mm, about 1.7 mm, about 1.8 mm, or about 1.9 mm. In some embodiments, the injectable gel particles have a size of at most about 1.1 mm, about 1.2 mm, about 1.3 mm, about 1.4 mm, about 1.5 mm, about 1.6 mm, about 1.7 mm, about 1.8 mm, about 1.9 mm, or about 2 mm.

Further, provided herein is a method of radiative protection of neighboring organs in a mammal, including man, comprising subepidermal administration at a site in said mammal where soft tissue radiative protection is desirable, of an implant comprising injectable gel particles of a viscoelastic medium, a major volume of said particles having a size, when subjected to a physiological salt solution, in the range of from 1 to 5 mm. In some embodiments, said administration is selected from the group consisting of subcutaneous administration, submuscular administration and supraperiostal administration. In some embodiments, said size is in the range of from 1 to 2.5 mm. In some embodiments, said site of radiative protection is selected from facial tissue and other tissues covered by exposed skin. In some embodiments, said size is in the range of from 2.5 to 5 mm. In some embodiments, said administration is a selected from the group consisting of single administration and multiple-layer administration.

Further, provided herein are injectable gel particles according to an embodiment herein for use as a medicament. Further, provided herein is an injectable radiative protection implant comprising injectable gel particles according to an embodiment herein for use as a medicament.

Further, provided herein is a method of using an injectable gel particles of a viscoelastic medium according to an embodiment herein. In some embodiments, the particles have an average size when subjected to a physiological salt solution, in the range of from 1 to 5 mm, for the manufacture of a medicament for therapeutic radiative protection in a mammal, including man, wherein said medicament is suitable for subepidermal administration according to an embodiment herein at a site in said mammal where therapeutic radiative protection is desirable.

Further provided herein are particles of a viscoelastic medium, which are injectable gel particles having a size, when subjected to a physiological salt solution, in the range of from 1 to 5 mm. The particles are useful in a radiative protection implant comprising particles of a viscoelastic medium, wherein a major volume of said particles are injectable gel particles having a specific size or range of sizes, when subjected to a physiological salt solution, in the range of from 1 to 5 mm. The implant, in turn, is useful in a method of radiative protection in a mammal, including man, comprising subepidermal administration at a site in said mammal where radiative protection is desirable, of an implant comprising injectable gel particles of a viscoelastic medium, a major volume of said particles having a size, when subjected to a physiological salt solution, in the range of from 1 to 5 mm.

Another aspect provided herein is a method of preventing or decreasing damage to a tissue proximate to a site of a radiotherapy in a subject undergoing the radiotherapy comprising an injection of a bioabsorbable viscoelastic medium at the site of the radiotherapy. In some embodiments, the viscoelastic medium comprises gel particles. In some embodiments, the gel particles comprise hyaluronic acid or derivatives thereof.

In some embodiments, the injection displaces the tissue by a distance of about 0.1 cm to about 10 cm. In some embodiments, the injection displaces the tissue by a distance of about 0.1 cm to about 0.2 cm, about 0.1 cm to about 0.5 cm, about 0.1 cm to about 1 cm, about 0.1 cm to about 2 cm, about 0.1 cm to about 3 cm, about 0.1 cm to about 4 cm, about 0.1 cm to about 5 cm, about 0.1 cm to about 6 cm, about 0.1 cm to about 7 cm, about 0.1 cm to about 8 cm, about 0.1 cm to about 10 cm, about 0.2 cm to about 0.5 cm, about 0.2 cm to about 1 cm, about 0.2 cm to about 2 cm, about 0.2 cm to about 3 cm, about 0.2 cm to about 4 cm, about 0.2 cm to about 5 cm, about 0.2 cm to about 6 cm, about 0.2 cm to about 7 cm, about 0.2 cm to about 8 cm, about 0.2 cm to about 10 cm, about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 10 cm, about 4 cm to about 5 cm, about 4 cm to about 6 cm, about 4 cm to about 7 cm, about 4 cm to about 8 cm, about 4 cm to about 10 cm, about 5 cm to about 6 cm, about 5 cm to about 7 cm, about 5 cm to about 8 cm, about 5 cm to about 10 cm, about 6 cm to about 7 cm, about 6 cm to about 8 cm, about 6 cm to about 10 cm, about 7 cm to about 8 cm, about 7 cm to about 10 cm, or about 8 cm to about 10 cm. In some embodiments, the injection displaces the tissue by a distance of about 0.1 cm, about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection displaces the tissue by a distance of at least about 0.1 cm, about 0.2 cm, about 0.5 cm, about 1 cm, about 2 cm, about 3 cm, about 4 cm, about 5 cm, about 6 cm, about 7 cm, or about 8 cm. In some embodiments, the injection displaces the tissue by a distance of at most about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the injection is performed by a needle having a gauge of about 10 to about 26. In some embodiments, the injection is performed by a needle having a gauge of about 10 to about 11, about 10 to about 12, about 10 to about 13, about 10 to about 14, about 10 to about 15, about 10 to about 16, about 10 to about 18, about 10 to about 20, about 10 to about 22, about 10 to about 24, about 10 to about 26, about 11 to about 12, about 11 to about 13, about 11 to about 14, about 11 to about 15, about 11 to about 16, about 11 to about 18, about 11 to about 20, about 11 to about 22, about 11 to about 24, about 11 to about 26, about 12 to about 13, about 12 to about 14, about 12 to about 15, about 12 to about 16, about 12 to about 18, about 12 to about 20, about 12 to about 22, about 12 to about 24, about 12 to about 26, about 13 to about 14, about 13 to about 15, about 13 to about 16, about 13 to about 18, about 13 to about 20, about 13 to about 22, about 13 to about 24, about 13 to about 26, about 14 to about 15, about 14 to about 16, about 14 to about 18, about 14 to about 20, about 14 to about 22, about 14 to about 24, about 14 to about 26, about 15 to about 16, about 15 to about 18, about 15 to about 20, about 15 to about 22, about 15 to about 24, about 15 to about 26, about 16 to about 18, about 16 to about 20, about 16 to about 22, about 16 to about 24, about 16 to about 26, about 18 to about 20, about 18 to about 22, about 18 to about 24, about 18 to about 26, about 20 to about 22, about 20 to about 24, about 20 to about 26, about 22 to about 24, about 22 to about 26, or about 24 to about 26. In some embodiments, the injection is performed by a needle having a gauge of about 10, about 11, about 12, about 13, about 14, about 15, about 16, about 18, about 20, about 22, about 24, or about 26. In some embodiments, the injection is performed by a needle having a gauge of at least about 10, about 11, about 12, about 13, about 14, about 15, about 16, about 18, about 20, about 22, or about 24. In some embodiments, the injection is performed by a needle having a gauge of at most about 11, about 12, about 13, about 14, about 15, about 16, about 18, about 20, about 22, about 24, or about 26.

In some embodiments, the concentration of the hyaluronic acid in the spacer material is about 1 mg/ml to about 100 mg/ml. In some embodiments, the concentration of the hyaluronic acid in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the hyaluronic acid in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the hyaluronic acid in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the hyaluronic acid in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the gel particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, the injection is subcutaneous or subepidermal. In some embodiments, migration of the viscoelastic medium is prevented or decreased. In some embodiments, the viscoelastic medium further comprises nanoparticles. In some embodiments, the nanoparticles comprise a precious metal. In some embodiments, a dose of the radiotherapy contacting the tissue proximate to the site of radiotherapy is reduced by about 10% to about 80%. In some embodiments, the site of the radiotherapy is selected from a group consisting of the subject's breast, head & neck, cervix, vagina, base of spine, skin, pancreas, liver, or lung. In some embodiments, the method further comprises an administration of hyaluronidase at the site of radiotherapy.

In some embodiments, the volume of the viscoelastic medium at the site of radiotherapy is reduced by about 1% to about 95%. In some embodiments, the volume of the viscoelastic medium at the site of radiotherapy is reduced by about 1% to about 5%, about 1% to about 10%, about 1% to about 15%, about 1% to about 20%, about 1% to about 30%, about 1% to about 40%, about 1% to about 50%, about 1% to about 60%, about 1% to about 70%, about 1% to about 80%, about 1% to about 95%, about 5% to about 10%, about 5% to about 15%, about 5% to about 20%, about 5% to about 30%, about 5% to about 40%, about 5% to about 50%, about 5% to about 60%, about 5% to about 70%, about 5% to about 80%, about 5% to about 95%, about 10% to about 15%, about 10% to about 20%, about 10% to about 30%, about 10% to about 40%, about 10% to about 50%, about 10% to about 60%, about 10% to about 70%, about 10% to about 80%, about 10% to about 95%, about 15% to about 20%, about 15% to about 30%, about 15% to about 40%, about 15% to about 50%, about 15% to about 60%, about 15% to about 70%, about 15% to about 80%, about 15% to about 95%, about 20% to about 30%, about 20% to about 40%, about 20% to about 50%, about 20% to about 60%, about 20% to about 70%, about 20% to about 80%, about 20% to about 95%, about 30% to about 40%, about 30% to about 50%, about 30% to about 60%, about 30% to about 70%, about 30% to about 80%, about 30% to about 95%, about 40% to about 50%, about 40% to about 60%, about 40% to about 70%, about 40% to about 80%, about 40% to about 95%, about 50% to about 60%, about 50% to about 70%, about 50% to about 80%, about 50% to about 95%, about 60% to about 70%, about 60% to about 80%, about 60% to about 95%, about 70% to about 80%, about 70% to about 95%, or about 80% to about 95%. In some embodiments, the volume of the viscoelastic medium at the site of radiotherapy is reduced by about 1%, about 5%, about 10%, about 15%, about 20% about 30%, about 40%, about 50%, about 60%, about 70%, about 80%, or about 95%. In some embodiments, the volume of the viscoelastic medium at the site of radiotherapy is reduced by at least about 1%, about 5%, about 10%, about 15%, about 20%, about 30%, about 40%, about 50%, about 60%, about 70%, or about 80%. In some embodiments, the volume of the viscoelastic medium at the site of radiotherapy is reduced by at most about 5%, about 10%, about 15% about 20%, about 30%, about 40%, about 50%, about 60%, about 70% about 80% or about 95%.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

Another aspect provided herein is a method of reducing a dose of radiotherapy to a tissue proximate to a site of a radiotherapy in a subject undergoing the radiotherapy comprising an injection of a bioabsorbable viscoelastic medium at the site of the radiotherapy. In some embodiments, the viscoelastic medium comprises gel particles. In some embodiments, the gel particles comprise hyaluronic acid or derivatives thereof.

In some embodiments, the injection displaces the tissue by a distance of about 0.1 cm to about 10 cm. In some embodiments, the injection displaces the tissue by a distance of about 0.1 cm to about 0.2 cm, about 0.1 cm to about 0.5 cm, about 0.1 cm to about 1 cm, about 0.1 cm to about 2 cm, about 0.1 cm to about 3 cm, about 0.1 cm to about 4 cm, about 0.1 cm to about 5 cm, about 0.1 cm to about 6 cm, about 0.1 cm to about 7 cm, about 0.1 cm to about 8 cm, about 0.1 cm to about 10 cm, about 0.2 cm to about 0.5 cm, about 0.2 cm to about 1 cm, about 0.2 cm to about 2 cm, about 0.2 cm to about 3 cm, about 0.2 cm to about 4 cm, about 0.2 cm to about 5 cm, about 0.2 cm to about 6 cm, about 0.2 cm to about 7 cm, about 0.2 cm to about 8 cm, about 0.2 cm to about 10 cm, about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 10 cm, about 4 cm to about 5 cm, about 4 cm to about 6 cm, about 4 cm to about 7 cm, about 4 cm to about 8 cm, about 4 cm to about 10 cm, about 5 cm to about 6 cm, about 5 cm to about 7 cm, about 5 cm to about 8 cm, about 5 cm to about 10 cm, about 6 cm to about 7 cm, about 6 cm to about 8 cm, about 6 cm to about 10 cm, about 7 cm to about 8 cm, about 7 cm to about 10 cm, or about 8 cm to about 10 cm. In some embodiments, the injection displaces the tissue by a distance of about 0.1 cm, about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection displaces the tissue by a distance of at least about 0.1 cm, about 0.2 cm, about 0.5 cm, about 1 cm, about 2 cm, about 3 cm, about 4 cm, about 5 cm, about 6 cm, about 7 cm, or about 8 cm. In some embodiments, the injection displaces the tissue by a distance of at most about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the concentration of the hyaluronic acid in the spacer material is about 1 mg/ml to about 100 mg/ml. In some embodiments, the concentration of the hyaluronic acid in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the hyaluronic acid in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the hyaluronic acid in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the hyaluronic acid in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the gel particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the gel particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the gel particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, the injection is subcutaneous or subepidermal. In some embodiments, migration of the viscoelastic medium is prevented or decreased. In some embodiments, the viscoelastic medium further comprises nanoparticles. In some embodiments, the nanoparticles comprise a precious metal. In some embodiments, the dose of radiotherapy is reduced by about 10% to about 80%. In some embodiments, the site of the radiotherapy is selected from a group consisting of the subject's breast, head & neck, cervix, uterus, ovaries, vagina, base of spine, skin, pancreas, liver, rectum, or lung. In some embodiments, the method further comprises an administration of hyaluronidase at the site of radiotherapy.

In some embodiments, the volume of the viscoelastic medium at the site of radiotherapy is reduced by about 1% to about 95%. In some embodiments, the volume of the viscoelastic medium at the site of radiotherapy is reduced by about 1% to about 5%, about 1% to about 10%, about 1% to about 15%, about 1% to about 20%, about 1% to about 30%, about 1% to about 40%, about 1% to about 50%, about 1% to about 60%, about 1% to about 70%, about 1% to about 80%, about 1% to about 95%, about 5% to about 10%, about 5% to about 15%, about 5% to about 20%, about 5% to about 30%, about 5% to about 40%, about 5% to about 50%, about 5% to about 60%, about 5% to about 70%, about 5% to about 80%, about 5% to about 95%, about 10% to about 15%, about 10% to about 20%, about 10% to about 30%, about 10% to about 40%, about 10% to about 50%, about 10% to about 60%, about 10% to about 70%, about 10% to about 80%, about 10% to about 95%, about 15% to about 20%, about 15% to about 30%, about 15% to about 40%, about 15% to about 50%, about 15% to about 60%, about 15% to about 70%, about 15% to about 80%, about 15% to about 95%, about 20% to about 30%, about 20% to about 40%, about 20% to about 50%, about 20% to about 60%, about 20% to about 70%, about 20% to about 80%, about 20% to about 95%, about 30% to about 40%, about 30% to about 50%, about 30% to about 60%, about 30% to about 70%, about 30% to about 80%, about 30% to about 95%, about 40% to about 50%, about 40% to about 60%, about 40% to about 70%, about 40% to about 80%, about 40% to about 95%, about 50% to about 60%, about 50% to about 70%, about 50% to about 80%, about 50% to about 95%, about 60% to about 70%, about 60% to about 80%, about 60% to about 95%, about 70% to about 80%, about 70% to about 95%, or about 80% to about 95%. In some embodiments, the volume of the viscoelastic medium at the site of radiotherapy is reduced by about 1%, about 5%, about 10%, about 15%, about 20% about 30%, about 40%, about 50%, about 60%, about 70%, about 80%, or about 95%. In some embodiments, the volume of the viscoelastic medium at the site of radiotherapy is reduced by at least about 1%, about 5%, about 10%, about 15%, about 20%, about 30%, about 40%, about 50%, about 60%, about 70%, or about 80%. In some embodiments, the volume of the viscoelastic medium at the site of radiotherapy is reduced by at most about 5%, about 10%, about 15% about 20%, about 30%, about 40%, about 50%, about 60%, about 70% about 80% or about 95%.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

Another aspect provided herein is a method of temporarily super-spacing a tissue proximate to a site of radiotherapy comprising injecting a formulation comprising cross-linked hyaluronic acid or derivatives thereof and an amount of degradable nanoparticles encapsulating hyaluronidase. In some embodiments, the amount of degradable nanoparticles encapsulating hyaluronidase is directly proportionate to a desired distance of super spacing relative to a desired time period of super spacing.

Another aspect provided herein is a method of treating cancer in subject suffering thereof comprising injecting a bioabsorbable viscoelastic medium in a blood vessel wherein the blood vessel is directly coupled to a tumor. In some embodiments, the viscoelastic medium comprises gel particles. In some embodiments, the gel particles comprise hyaluronic acid or derivatives thereof.

In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the injection is performed by a needle having a gauge of about 10 to about 26. In some embodiments, the injection is performed by a needle having a gauge of about 10 to about 11, about 10 to about 12, about 10 to about 13, about 10 to about 14, about 10 to about 15, about 10 to about 16, about 10 to about 18, about 10 to about 20, about 10 to about 22, about 10 to about 24, about 10 to about 26, about 11 to about 12, about 11 to about 13, about 11 to about 14, about 11 to about 15, about 11 to about 16, about 11 to about 18, about 11 to about 20, about 11 to about 22, about 11 to about 24, about 11 to about 26, about 12 to about 13, about 12 to about 14, about 12 to about 15, about 12 to about 16, about 12 to about 18, about 12 to about 20, about 12 to about 22, about 12 to about 24, about 12 to about 26, about 13 to about 14, about 13 to about 15, about 13 to about 16, about 13 to about 18, about 13 to about 20, about 13 to about 22, about 13 to about 24, about 13 to about 26, about 14 to about 15, about 14 to about 16, about 14 to about 18, about 14 to about 20, about 14 to about 22, about 14 to about 24, about 14 to about 26, about 15 to about 16, about 15 to about 18, about 15 to about 20, about 15 to about 22, about 15 to about 24, about 15 to about 26, about 16 to about 18, about 16 to about 20, about 16 to about 22, about 16 to about 24, about 16 to about 26, about 18 to about 20, about 18 to about 22, about 18 to about 24, about 18 to about 26, about 20 to about 22, about 20 to about 24, about 20 to about 26, about 22 to about 24, about 22 to about 26, or about 24 to about 26. In some embodiments, the injection is performed by a needle having a gauge of about 10, about 11, about 12, about 13, about 14, about 15, about 16, about 18, about 20, about 22, about 24, or about 26. In some embodiments, the injection is performed by a needle having a gauge of at least about 10, about 11, about 12, about 13, about 14, about 15, about 16, about 18, about 20, about 22, or about 24. In some embodiments, the injection is performed by a needle having a gauge of at most about 11, about 12, about 13, about 14, about 15, about 16, about 18, about 20, about 22, about 24, or about 26.

In some embodiments, the concentration of the hyaluronic acid in the spacer material is about 1 mg/ml to about 100 mg/ml. In some embodiments, the concentration of the hyaluronic acid in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the hyaluronic acid in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the hyaluronic acid in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the hyaluronic acid in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the gel particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the gel particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the gel particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the gel particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, blood flow to the tumor is prevented or decreased. In some embodiments, migration of the viscoelastic medium is prevented or decreased. In some embodiments, the method further comprises an administration of hyaluronidase at the site of radiotherapy.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

In some embodiments, the method further comprises excising the remaining tumor cells from the subject.

Another aspect provided herein is a formulation comprising cross-linked hyaluronic acid and a radiopaque compound selected from the group consisting of iohexol, metrizamide, iopamidol, 3,5-bis(acetylamino)-2,4,6-triiodobenzoic acid, meglumine diatrizoate, iopentol, iopromide, triiodobenzoic acid, erythrosine, and ioversol. In some embodiments, the formulation is used as a fiducial marker.

Another aspect provided herein is a method of preventing or decreasing damage to a tissue proximate to a site of a radiotherapy in a subject undergoing the radiotherapy comprising an injection of a bioabsorbable viscoelastic medium at the site of the radiotherapy. In some embodiments, the viscoelastic medium comprises gel particles. In some embodiments, the gel particles comprise polyethylene glycol or derivatives thereof.

In some embodiments, the injection displaces the tissue by a distance of about 0.1 cm to about 10 cm. In some embodiments, the injection displaces the tissue by a distance of about 0.1 cm to about 0.2 cm, about 0.1 cm to about 0.5 cm, about 0.1 cm to about 1 cm, about 0.1 cm to about 2 cm, about 0.1 cm to about 3 cm, about 0.1 cm to about 4 cm, about 0.1 cm to about 5 cm, about 0.1 cm to about 6 cm, about 0.1 cm to about 7 cm, about 0.1 cm to about 8 cm, about 0.1 cm to about 10 cm, about 0.2 cm to about 0.5 cm, about 0.2 cm to about 1 cm, about 0.2 cm to about 2 cm, about 0.2 cm to about 3 cm, about 0.2 cm to about 4 cm, about 0.2 cm to about 5 cm, about 0.2 cm to about 6 cm, about 0.2 cm to about 7 cm, about 0.2 cm to about 8 cm, about 0.2 cm to about 10 cm, about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 10 cm, about 4 cm to about 5 cm, about 4 cm to about 6 cm, about 4 cm to about 7 cm, about 4 cm to about 8 cm, about 4 cm to about 10 cm, about 5 cm to about 6 cm, about 5 cm to about 7 cm, about 5 cm to about 8 cm, about 5 cm to about 10 cm, about 6 cm to about 7 cm, about 6 cm to about 8 cm, about 6 cm to about 10 cm, about 7 cm to about 8 cm, about 7 cm to about 10 cm, or about 8 cm to about 10 cm. In some embodiments, the injection displaces the tissue by a distance of about 0.1 cm, about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection displaces the tissue by a distance of at least about 0.1 cm, about 0.2 cm, about 0.5 cm, about 1 cm, about 2 cm, about 3 cm, about 4 cm, about 5 cm, about 6 cm, about 7 cm, or about 8 cm. In some embodiments, the injection displaces the tissue by a distance of at most about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the injection is performed by a needle having a gauge of about 10 to about 26. In some embodiments, the injection is performed by a needle having a gauge of about 10 to about 11, about 10 to about 12, about 10 to about 13, about 10 to about 14, about 10 to about 15, about 10 to about 16, about 10 to about 18, about 10 to about 20, about 10 to about 22, about 10 to about 24, about 10 to about 26, about 11 to about 12, about 11 to about 13, about 11 to about 14, about 11 to about 15, about 11 to about 16, about 11 to about 18, about 11 to about 20, about 11 to about 22, about 11 to about 24, about 11 to about 26, about 12 to about 13, about 12 to about 14, about 12 to about 15, about 12 to about 16, about 12 to about 18, about 12 to about 20, about 12 to about 22, about 12 to about 24, about 12 to about 26, about 13 to about 14, about 13 to about 15, about 13 to about 16, about 13 to about 18, about 13 to about 20, about 13 to about 22, about 13 to about 24, about 13 to about 26, about 14 to about 15, about 14 to about 16, about 14 to about 18, about 14 to about 20, about 14 to about 22, about 14 to about 24, about 14 to about 26, about 15 to about 16, about 15 to about 18, about 15 to about 20, about 15 to about 22, about 15 to about 24, about 15 to about 26, about 16 to about 18, about 16 to about 20, about 16 to about 22, about 16 to about 24, about 16 to about 26, about 18 to about 20, about 18 to about 22, about 18 to about 24, about 18 to about 26, about 20 to about 22, about 20 to about 24, about 20 to about 26, about 22 to about 24, about 22 to about 26, or about 24 to about 26. In some embodiments, the injection is performed by a needle having a gauge of about 10, about 11, about 12, about 13, about 14, about 15, about 16, about 18, about 20, about 22, about 24, or about 26. In some embodiments, the injection is performed by a needle having a gauge of at least about 10, about 11, about 12, about 13, about 14, about 15, about 16, about 18, about 20, about 22, or about 24. In some embodiments, the injection is performed by a needle having a gauge of at most about 11, about 12, about 13, about 14, about 15, about 16, about 18, about 20, about 22, about 24, or about 26.

In some embodiments, the concentration of the polyethylene glycol in the spacer material is about 1 mg/ml to about 100 mg/ml. In some embodiments, the concentration of the polyethylene glycol in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the polyethylene glycol in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the polyethylene glycol in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the polyethylene glycol in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the gel particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the gel particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the gel particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the gel particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, the injection is subcutaneous or subepidermal. In some embodiments, migration of the viscoelastic medium is prevented or decreased. In some embodiments, the viscoelastic medium further comprises nanoparticles. In some embodiments, the nanoparticles comprise a precious metal. In some embodiments, a dose of the radiotherapy contacting the tissue proximate to the site of radiotherapy is reduced by about 10% to about 80%. In some embodiments, the site of the radiotherapy is selected from a group consisting of the subject's breast, head & neck, cervix, vagina, base of spine, skin, pancreas, liver, or lung. In some embodiments, the method further comprises an administration of hyaluronidase at the site of radiotherapy.

In some embodiments, the volume of the viscoelastic medium at the site of radiotherapy is reduced by about 1% to about 95%. In some embodiments, the volume of the viscoelastic medium at the site of radiotherapy is reduced by about 1% to about 5%, about 1% to about 10%, about 1% to about 15%, about 1% to about 20%, about 1% to about 30%, about 1% to about 40%, about 1% to about 50%, about 1% to about 60%, about 1% to about 70%, about 1% to about 80%, about 1% to about 95%, about 5% to about 10%, about 5% to about 15%, about 5% to about 20%, about 5% to about 30%, about 5% to about 40%, about 5% to about 50%, about 5% to about 60%, about 5% to about 70%, about 5% to about 80%, about 5% to about 95%, about 10% to about 15%, about 10% to about 20%, about 10% to about 30%, about 10% to about 40%, about 10% to about 50%, about 10% to about 60%, about 10% to about 70%, about 10% to about 80%, about 10% to about 95%, about 15% to about 20%, about 15% to about 30%, about 15% to about 40%, about 15% to about 50%, about 15% to about 60%, about 15% to about 70%, about 15% to about 80%, about 15% to about 95%, about 20% to about 30% about 20% to about 40%, about 20% to about 50%, about 20% to about 60%, about 20% to about 70%, about 20% to about 80%, about 20% to about 95%, about 30% to about 40%, about 30% to about 50%, about 30% to about 60%, about 30% to about 70%, about 30% to about 80%, about 30% to about 95%, about 40% to about 50%, about 40% to about 60%, about 40% to about 70%, about 40% to about 80%, about 40% to about 95%, about 50% to about 60%, about 50% to about 70%, about 50% to about 80%, about 50% to about 95%, about 60% to about 70%, about 60% to about 80%, about 60% to about 95%, about 70% to about 80%, about 70% to about 95%, or about 80% to about 95%. In some embodiments, the volume of the viscoelastic medium at the site of radiotherapy is reduced by about 1%, about 5%, about 10%, about 15%, about 20% about 30%, about 40%, about 50%, about 60%, about 70%, about 80%, or about 95%. In some embodiments, the volume of the viscoelastic medium at the site of radiotherapy is reduced by at least about 1%, about 5%, about 10%, about 15%, about 20%, about 30%, about 40%, about 50%, about 60%, about 70%, or about 80%. In some embodiments, the volume of the viscoelastic medium at the site of radiotherapy is reduced by at most about 5%, about 10%, about 15% about 20%, about 30%, about 40%, about 50%, about 60%, about 70% about 80% or about 95%.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

Another aspect provided herein is a method of reducing a dose of radiotherapy to a tissue proximate to a site of a radiotherapy in a subject undergoing the radiotherapy comprising an injection of a bioabsorbable viscoelastic medium at the site of the radiotherapy. In some embodiments, the viscoelastic medium comprises gel particles. In some embodiments, the gel particles comprise polyethylene glycol or derivatives thereof.

In some embodiments, the injection displaces the tissue by a distance of about 0.1 cm to about 10 cm. In some embodiments, the injection displaces the tissue by a distance of about 0.1 cm to about 0.2 cm, about 0.1 cm to about 0.5 cm, about 0.1 cm to about 1 cm, about 0.1 cm to about 2 cm, about 0.1 cm to about 3 cm, about 0.1 cm to about 4 cm, about 0.1 cm to about 5 cm, about 0.1 cm to about 6 cm, about 0.1 cm to about 7 cm, about 0.1 cm to about 8 cm, about 0.1 cm to about 10 cm, about 0.2 cm to about 0.5 cm, about 0.2 cm to about 1 cm, about 0.2 cm to about 2 cm, about 0.2 cm to about 3 cm, about 0.2 cm to about 4 cm, about 0.2 cm to about 5 cm, about 0.2 cm to about 6 cm, about 0.2 cm to about 7 cm, about 0.2 cm to about 8 cm, about 0.2 cm to about 10 cm, about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 10 cm, about 4 cm to about 5 cm, about 4 cm to about 6 cm, about 4 cm to about 7 cm, about 4 cm to about 8 cm, about 4 cm to about 10 cm, about 5 cm to about 6 cm, about 5 cm to about 7 cm, about 5 cm to about 8 cm, about 5 cm to about 10 cm, about 6 cm to about 7 cm, about 6 cm to about 8 cm, about 6 cm to about 10 cm, about 7 cm to about 8 cm, about 7 cm to about 10 cm, or about 8 cm to about 10 cm. In some embodiments, the injection displaces the tissue by a distance of about 0.1 cm, about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection displaces the tissue by a distance of at least about 0.1 cm, about 0.2 cm, about 0.5 cm, about 1 cm, about 2 cm, about 3 cm, about 4 cm, about 5 cm, about 6 cm, about 7 cm, or about 8 cm. In some embodiments, the injection displaces the tissue by a distance of at most about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the concentration of the polyethylene glycol in the spacer material is about 1 mg/ml to about 100 mg/ml. In some embodiments, the concentration of the polyethylene glycol in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the polyethylene glycol in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the polyethylene glycol in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the polyethylene glycol in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the gel particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the gel particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the gel particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the gel particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, the injection is subcutaneous or subepidermal. In some embodiments, migration of the viscoelastic medium is prevented or decreased. In some embodiments, the viscoelastic medium further comprises nanoparticles. In some embodiments, the nanoparticles comprise a precious metal. In some embodiments, the dose of radiotherapy is reduced by about 10% to about 80%. In some embodiments, the site of the radiotherapy is selected from a group consisting of the subject's breast, head & neck, cervix, vagina, base of spine, skin, pancreas, liver, rectum, or lung. In some embodiments, the method further comprises an administration of hyaluronidase at the site of radiotherapy.

In some embodiments, the volume of the viscoelastic medium at the site of radiotherapy is reduced by about 1% to about 95%. In some embodiments, the volume of the viscoelastic medium at the site of radiotherapy is reduced by about 1% to about 5%, about 1% to about 10%, about 1% to about 15%, about 1% to about 20%, about 1% to about 30%, about 1% to about 40%, about 1% to about 50%, about 1% to about 60%, about 1% to about 70%, about 1% to about 80%, about 1% to about 95%, about 5% to about 10%, about 5% to about 15%, about 5% to about 20%, about 5% to about 30%, about 5% to about 40%, about 5% to about 50%, about 5% to about 60%, about 5% to about 70%, about 5% to about 80%, about 5% to about 95%, about 10% to about 15%, about 10% to about 20%, about 10% to about 30%, about 10% to about 40%, about 10% to about 50%, about 10% to about 60%, about 10% to about 70%, about 10% to about 80%, about 10% to about 95%, about 15% to about 20%, about 15% to about 30%, about 15% to about 40%, about 15% to about 50%, about 15% to about 60%, about 15% to about 70%, about 15% to about 80%, about 15% to about 95%, about 20% to about 30% about 20% to about 40%, about 20% to about 50%, about 20% to about 60%, about 20% to about 70%, about 20% to about 80%, about 20% to about 95%, about 30% to about 40%, about 30% to about 50%, about 30% to about 60%, about 30% to about 70%, about 30% to about 80%, about 30% to about 95%, about 40% to about 50%, about 40% to about 60%, about 40% to about 70%, about 40% to about 80%, about 40% to about 95%, about 50% to about 60%, about 50% to about 70%, about 50% to about 80%, about 50% to about 95%, about 60% to about 70%, about 60% to about 80%, about 60% to about 95%, about 70% to about 80%, about 70% to about 95%, or about 80% to about 95%. In some embodiments, the volume of the viscoelastic medium at the site of radiotherapy is reduced by about 1%, about 5%, about 10%, about 15%, about 20% about 30%, about 40%, about 50%, about 60%, about 70%, about 80%, or about 95%. In some embodiments, the volume of the viscoelastic medium at the site of radiotherapy is reduced by at least about 1%, about 5%, about 10%, about 15%, about 20%, about 30%, about 40%, about 50%, about 60%, about 70%, or about 80%. In some embodiments, the volume of the viscoelastic medium at the site of radiotherapy is reduced by at most about 5%, about 10%, about 15% about 20%, about 30%, about 40%, about 50%, about 60%, about 70% about 80% or about 95%.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

Another aspect provided herein is a method of temporarily super-spacing a tissue proximate to a site of radiotherapy comprising injecting a formulation comprising cross-linked polyethylene glycol or derivatives thereof and an amount of degradable nanoparticles encapsulating hyaluronidase. In some embodiments, the amount of degradable nanoparticles encapsulating hyaluronidase is directly proportionate to a desired distance of super spacing relative to a desired time period of super spacing.

Another aspect provided herein is a method of treating cancer in subject suffering thereof comprising injecting a bioabsorbable viscoelastic medium in a blood vessel wherein the blood vessel is directly coupled to a tumor. In some embodiments, the viscoelastic medium comprises gel particles. In some embodiments, the gel particles comprise polyethylene glycol or derivatives thereof.

In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the injection is performed by a needle having a gauge of about 10 to about 26. In some embodiments, the injection is performed by a needle having a gauge of about 10 to about 11, about 10 to about 12, about 10 to about 13, about 10 to about 14, about 10 to about 15, about 10 to about 16, about 10 to about 18, about 10 to about 20, about 10 to about 22, about 10 to about 24, about 10 to about 26, about 11 to about 12, about 11 to about 13, about 11 to about 14, about 11 to about 15, about 11 to about 16, about 11 to about 18, about 11 to about 20, about 11 to about 22, about 11 to about 24, about 11 to about 26, about 12 to about 13, about 12 to about 14, about 12 to about 15, about 12 to about 16, about 12 to about 18, about 12 to about 20, about 12 to about 22, about 12 to about 24, about 12 to about 26, about 13 to about 14, about 13 to about 15, about 13 to about 16, about 13 to about 18, about 13 to about 20, about 13 to about 22, about 13 to about 24, about 13 to about 26, about 14 to about 15, about 14 to about 16, about 14 to about 18, about 14 to about 20, about 14 to about 22, about 14 to about 24, about 14 to about 26, about 15 to about 16, about 15 to about 18, about 15 to about 20, about 15 to about 22, about 15 to about 24, about 15 to about 26, about 16 to about 18, about 16 to about 20, about 16 to about 22, about 16 to about 24, about 16 to about 26, about 18 to about 20, about 18 to about 22, about 18 to about 24, about 18 to about 26, about 20 to about 22, about 20 to about 24, about 20 to about 26, about 22 to about 24, about 22 to about 26, or about 24 to about 26. In some embodiments, the injection is performed by a needle having a gauge of about 10, about 11, about 12, about 13, about 14, about 15, about 16, about 18, about 20, about 22, about 24, or about 26. In some embodiments, the injection is performed by a needle having a gauge of at least about 10, about 11, about 12, about 13, about 14, about 15, about 16, about 18, about 20, about 22, or about 24. In some embodiments, the injection is performed by a needle having a gauge of at most about 11, about 12, about 13, about 14, about 15, about 16, about 18, about 20, about 22, about 24, or about 26.

In some embodiments, the concentration of the polyethylene glycol in the spacer material is about 1 mg/ml to about 100 mg/ml. In some embodiments, the concentration of the polyethylene glycol in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the polyethylene glycol in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the polyethylene glycol in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the polyethylene glycol in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the gel particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, blood flow to the tumor is prevented or decreased. In some embodiments, migration of the viscoelastic medium is prevented or decreased. In some embodiments, the method further comprises an administration of hyaluronidase at the site of radiotherapy.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

In some embodiments, the method further comprises excising the remaining tumor cells from the subject.

Another aspect provided herein is a formulation comprising cross-linked polyethylene glycol and a radiopaque compound selected from the group consisting of iohexol, metrizamide, iopamidol, 3,5-bis(acetylamino)-2,4,6-triiodobenzoic acid, meglumine diatrizoate, iopentol, iopromide, triiodobenzoic acid, erythrosine, and ioversol. In some embodiments, the formulation is used as a fiducial marker.

Some embodiments herein disclose a composition comprising: a viscoelastic medium; and a first visual additive, wherein said first visual additive comprises a metal, and wherein said metal has a particle diameter of greater than or equal to 80 micrometers (μm). In some embodiments, said metal is a precious metal. In some embodiments, said metal or said precious metal is chosen from the group consisting of gold (Au), iodine (I), gadolinium (Gd), iron (Fe), barium (Ba), calcium (Ca), magnesium (Mg), and combinations thereof. In some embodiments, said metal or precious metal is an isotope of gold (Au), iodine (I), gadolinium (Gd), iron (Fe), barium (Ba), calcium (Ca), magnesium (Mg), or combinations thereof. In some embodiments, said metal or precious metal is a powder. In some embodiments, said first visual additive has a radiographic density of between about 1.0 g/cm3 and 2.0 g/cm3. In some embodiments, half of said first visual additive is configured to disperse within a tissue within nine months. In some embodiments, said particle diameter of said metal or precious metal is between about 80 μm and about 120 μm. In some embodiments, said particle diameter of said metal or precious metal is about 100 μm. In some embodiments, said first visual additive further comprises one or more microbubbles. In some embodiments, the composition further comprising a second visual additive. In some embodiments, said second visual additive is different than said first visual additive. In some embodiments, said second visual additive comprises one or more microbubbles. In some embodiments, said first visual additive has a concentration within said viscoelastic medium of greater than 5 milligrams per milliliter (mg/ml). In some embodiments, said first visual additive has a concentration within said viscoelastic medium of less than 90 mg/ml. In some embodiments, said first visual additive has a concentration within said viscoelastic medium between 15 mg/ml and 30 mg/ml. In some embodiments, said metal is between about 0.5 wt % and about 9.0 wt % of said composition. In some embodiments, said metal or precious metal is between about 0.015 wt % and about 1.5 wt % of said composition. In some embodiments, said viscoelastic medium comprises a volume of about 1 milliliter (ml) to about 50 ml. In some embodiments, said composition is configured to be biodegradable. In some embodiments, said composition is configured to be present on an imaging modality for at least 9 months. In some embodiments, said composition is configured to not substantially migrate prior to or during imaging. In some embodiments, said first visual additive is configured to not substantially migrate prior to or during imaging. In some embodiments, said composition is configured to be disposed within a subject. In some embodiments, said subject is in need of radiography. In some embodiments, said composition is configured to be disposed through injection. In some embodiments, composition is configured to be disposed subcutaneously or subepidermally. In some embodiments, composition is configured to be disposed within a tissue site. In some embodiments, said tissue site comprises one or more of, a fat tissue, a muscle tissue, and organ tissue, or a combination thereof. In some embodiments, composition is configured to be imaged on one or more modalities. In some embodiments, said one or more modalities comprise X-Ray, MRI, CT, CBCT, ultrasound, PET, SPECT or a combination thereof. In some embodiments, said composition being configured to be imaged on said one or more modalities comprises said composition being configured to be imaged in real time on said one or more modalities. In some embodiments, said composition is configured to be imaged within 30 min, within 90 min, within 4 hours, within 8 hours, or within 4 days of disposition. In some embodiments, said viscoelastic medium comprises hyaluronic acid, polyethylene glycol, or dextranomers. In some embodiments, said viscoelastic medium comprises hyaluronic acid, polyethylene glycol, or dextranomers at a concentration between about 5 mg/ml to about 100 mg/ml. In some embodiments, said viscoelastic medium comprises gel particles at a size range of about 0.08 mm to about 5 mm. In some embodiments, said gel particles comprise said metal particles. In some embodiments, said viscoelastic medium comprises non-animal stabilized hyaluronic acid (“NASHA”). In some embodiments, said viscoelastic medium expands within said tissue site to less than 10% of an original disposition volume. In some embodiments, said viscoelastic medium is injected one time every six months. In some embodiments, said viscoelastic medium is completely resorbed within 20 months. In some embodiments, said viscoelastic medium is completely resorbed within 16 months. In some embodiments, said viscoelastic medium is completely resorbed within 12 months.

Certain embodiments disclosed herein are directed towards a method of visualizing a tissue, space or location of a radiographic subject, comprising disposing an imaging contrast composition, said composition comprising: a viscoelastic medium; and a first visual additive, wherein said first visual additive is a metal, and wherein said metal has a particle diameter greater than about 80 micrometers. In some embodiments, the method further comprises: disposing the composition within a first tissue site, and imaging said composition within said first tissue site. In some embodiments, said metal has a particle diameter of greater than 100 micrometers. In some embodiments, the particle diameter of said metal is large enough such that the metal is not absorbed by tissue in the subject receiving radiation therapy.

Certain embodiments disclosed herein are directed towards a method of spacing a first tissue site of a subject from a second tissue site of said subject, the method comprising: disposing a composition in a space between said first tissue site and said second tissue site, wherein said composition comprises a viscoelastic medium and a first visual additive, wherein said first visual additive is a metal, and wherein said metal has a particle diameter of greater than 80 micrometers (μm). In some embodiments, said first visual additive has a concentration within said viscoelastic medium, wherein the concentration results in a contrast to noise ratio of about 0.1 to about 10. In some embodiments, said metal is a precious metal. In some embodiments, said metal or said precious metal is chosen from the group consisting of gold (Au), iodine (I), gadolinium (Gd), iron (Fe), barium (Ba), calcium (Ca), magnesium (Mg), and combinations thereof. In some embodiments, said metal or precious metal is an isotope of gold (Au), iodine (I), gadolinium (Gd), iron (Fe), barium (Ba), calcium (Ca), magnesium (Mg), or combinations thereof. In some embodiments, said metal or precious metal is a powder. In some embodiments, said first visual additive has a radiographic density of between about 1.0 g/cm3 and 2.0 g/cm3. In some embodiments, half of said first visual additive is configured to disperse within a tissue within nine months. In some embodiments, said particle diameter of said metal or precious metal is between about 80 μm and about 120 μm. In some embodiments, said particle diameter of said metal or precious metal is about 100 μm. In some embodiments, said first visual additive further comprises one or more microbubbles. In some embodiments, the composition further comprises a second visual additive. In some embodiments, said second visual additive is different than said first visual additive. In some embodiments, said second visual additive comprises one or more microbubbles. In some embodiments, said first visual additive has a concentration within said viscoelastic medium of greater than 5 milligrams per milliliter (mg/ml). In some embodiments, said first visual additive has a concentration within said viscoelastic medium of less than 90 mg/ml. In some embodiments, said first visual additive has a concentration within said viscoelastic medium between 15 mg/ml and 30 mg/ml. In some embodiments, said metal is between about 0.5 wt % and about 9.0 wt % of said composition. In some embodiments, said metal or precious metal is between about 0.015 wt % and about 1.5 wt % of said composition. In some embodiments, said viscoelastic medium comprises a volume of about 1 milliliter (ml) to about 50 ml. In some embodiments, said composition is configured to be biodegradable. In some embodiments, said composition is configured to be detectable on an imaging modality for at least 9 months. In some embodiments, said composition is configured to not substantially migrate prior to or during imaging, wherein the imaging occurs about 3 months to about 9 months the composition is disposed. In some embodiments, said first visual additive is configured to not substantially migrate prior to or during imaging, wherein the imaging occurs about 3 months to about 9 months the composition is disposed. In some embodiments, said composition is configured to be disposed within a subject. The method of claim 72, wherein said subject is in need of radiography. In some embodiments, said composition is configured to be disposed through injection. In some embodiments, said composition is configured to be disposed subcutaneously or subepidermally. In some embodiments, said composition is configured to be disposed within said first tissue site. In some embodiments, said first tissue site comprises one or more of, a fat tissue, a muscle tissue, and organ tissue, or a combination thereof. In some embodiments, said composition is configured to be imaged on one or more modalities. In some embodiments, said one or more modalities comprise X-Ray, MRI, CT, CBCT, ultrasound, PET, SPECT or a combination thereof. In some embodiments, said composition being configured to be imaged on said one or more modalities comprises said composition being configured to be imaged in real-time on said one or more modalities. In some embodiments, said composition is configured to be imaged within 30 min, within 90 min, within 4 hours, within 8 hours, or within 4 days of disposition. In some embodiments, said viscoelastic medium comprises hyaluronic acid, polyethylene glycol, or dextranomers. In some embodiments, said viscoelastic medium comprises hyaluronic acid, polyethylene glycol, or dextranomers at a concentration between about 5 mg/ml to about 100 mg/ml. In some embodiments, said viscoelastic medium comprises gel particles at a size range of about 0.08 mm to about 5 mm. In some embodiments, said gel particles comprise said metal particles. In some embodiments, said viscoelastic medium comprises non-animal stabilized hyaluronic acid (“NASHA”). In some embodiments, said viscoelastic medium expands within said first tissue site to less than 10% of an original disposition volume. In some embodiments, said viscoelastic medium is injected one time every six months. In some embodiments, said viscoelastic medium is completely resorbed within 20 months. In some embodiments, said viscoelastic medium is completely resorbed within 16 months. In some embodiments, said viscoelastic medium is completely resorbed within 12 months.

Certain embodiments disclosed herein are directed towards a method of treating cancer in a subject in need thereof, the method comprising: identifying a tumor within the subject; administering a composition adjacent to the tumor, the composition comprising: a viscoelastic medium; and a first visual additive, wherein said first visual additive is a metal, and wherein said metal has a particle diameter greater than about 80 micrometers; and applying a dose of radiation to the tumor. In some embodiments, said first visual additive has a concentration within said viscoelastic medium, wherein the concentration results in a contrast to noise ratio of about 0.1 to about 40. In some embodiments, said metal is a precious metal. In some embodiments, said metal or said precious metal is chosen from the group consisting of gold (Au), iodine (I), gadolinium (Gd), iron (Fe), barium (Ba), calcium (Ca), magnesium (Mg), and combinations thereof. In some embodiments, said metal or precious metal is an isotope of gold (Au), iodine (I), gadolinium (Gd), iron (Fe), barium (Ba), calcium (Ca), magnesium (Mg), or combinations thereof. In some embodiments, said metal or precious metal is a powder. In some embodiments, said first visual additive has a radiographic density of between about 1.0 g/cm3 and 2.0 g/cm3. In some embodiments, half of said first visual additive is configured to disperse within a tissue within nine months. In some embodiments, said particle diameter of said metal or precious metal is between about 80 μm and about 120 μm. In some embodiments, said particle diameter of said metal or precious metal is about 100 μm. In some embodiments, said first visual additive further comprises one or more microbubbles. In some embodiments, the composition comprises a second visual additive. In some embodiments, said second visual additive is different than said first visual additive. In some embodiments, said second visual additive comprises one or more microbubbles. In some embodiments, said first visual additive has a concentration within said viscoelastic medium of greater than 5 milligrams per milliliter (mg/ml). In some embodiments, said first visual additive has a concentration within said viscoelastic medium of less than 90 mg/ml. In some embodiments, said first visual additive has a concentration within said viscoelastic medium between 15 mg/ml and 30 mg/ml. In some embodiments, said metal is between about 0.5 wt % and about 9.0 wt % of said composition. In some embodiments, said metal or precious metal is between about 0.015 wt % and about 1.5 wt % of said composition. In some embodiments, said viscoelastic medium comprises a volume of about 1 milliliter (ml) to about 50 ml. In some embodiments, said composition is configured to be biodegradable. In some embodiments, said composition is configured to be detectable on an imaging modality for at least 9 months. In some embodiments, said composition is configured to not substantially migrate prior to or during imaging, wherein the imaging occurs about 3 months to about 9 months the composition is disposed. In some embodiments, said first visual additive is configured to not substantially migrate prior to or during imaging, wherein the imaging occurs about 3 months to about 9 months the composition is disposed. In some embodiments, said composition is configured to be disposed within the subject. In some embodiments, said subject is in need of radiography. In some embodiments, said composition is configured to be disposed through injection. In some embodiments, said composition is configured to be disposed subcutaneously or subepidermally. In some embodiments, said composition is configured to be disposed within said first tissue site. In some embodiments, said first tissue site comprises one or more of, a fat tissue, a muscle tissue, and organ tissue, or a combination thereof. In some embodiments, said composition is configured to be imaged on one or more modalities. In some embodiments, said one or more modalities comprise X-Ray, MRI, CT, CBCT, ultrasound, PET, SPECT or a combination thereof. In some embodiments, said composition being configured to be imaged on said one or more modalities comprises said composition being configured to be imaged in real-time on said one or more modalities. In some embodiments, said composition is configured to be imaged within 30 min, within 90 min, within 4 hours, within 8 hours, or within 4 days of disposition. In some embodiments, said viscoelastic medium comprises hyaluronic acid, polyethylene glycol, or dextranomers. In some embodiments, said viscoelastic medium comprises hyaluronic acid, polyethylene glycol, or dextranomers at a concentration between about 5 mg/ml to about 100 mg/ml. In some embodiments, said viscoelastic medium comprises gel particles at a size range of about 0.08 mm to about 5 mm. In some embodiments, said gel particles comprise said metal particles. In some embodiments, said viscoelastic medium comprises non-animal stabilized hyaluronic acid (“NASHA”). In some embodiments, said viscoelastic medium expands within said first tissue site to less than 10% of an original disposition volume. In some embodiments, said viscoelastic medium is injected one time every six months. In some embodiments, said viscoelastic medium is completely resorbed within 20 months. In some embodiments, said viscoelastic medium is completely resorbed within 16 months. In some embodiments, said viscoelastic medium is completely resorbed within 12 months.

### BRIEF DESCRIPTION OF THE DRAWINGS

The patent application contains at least one drawing executed in color. Copies of this patent or patent application with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is an exemplary image of an injected area of a mastectomy sample; according to an embodiment herein;

FIG. 1B is an exemplary image of injecting a spacer into a mastectomy sample using ultrasound guidance; according to an embodiment herein;

FIG. 2A is an ultrasound exemplary image of gland and adipose tissue within a mastectomy sample; according to an embodiment herein;

FIG. 2B is an ultrasound exemplary image of a spacer, gland tissue, and adipose tissue within a mastectomy sample; according to an embodiment herein;

FIG. 3A is an exemplary computed tomography (CT) scan of a hyaluronic acid (HA) spacer within a mastectomy sample; according to an embodiment herein;

FIG. 3B is an exemplary ultrasound image of a HA spacer within a mastectomy sample; according to an embodiment herein;

FIG. 3C is an exemplary CT scan of a polyethylene glycol (PEG) spacer within a mastectomy sample; according to an embodiment herein;

FIG. 3D is an exemplary CT scan of a PGA spacer within a mastectomy sample; according to an embodiment herein;

FIG. 4A is an exemplary image of an image of a simulated permanent breast seed implant (PBSI) brachytherapy planning of a hyaluronic acid (HA) spacer within a mastectomy sample; according to an embodiment herein;

FIG. 4B is an exemplary ultrasound image of a HA spacer within a mastectomy sample; according to an embodiment herein;

FIG. 5A is an exemplary image of a computed tomography scan before hydrogel spacer injection between the head of the pancreas and duodenum, according to an embodiment herein;

FIG. 5B is an exemplary image of a computed tomography scan after hydrogel spacer injection between the head of the pancreas and duodenum, according to an embodiment herein;

FIG. 5C is an exemplary image of a gross histologic specimen after hydrogel spacer injection between the head of the pancreas and duodenum, according to an embodiment herein;

FIG. 5D is an exemplary image of a computed tomography scan before a laparotomy hydrogel spacer injection between the head of the pancreas and duodenum, according to an embodiment herein;

FIG. 5E is an exemplary image of a computed tomography scan after a laparotomy hydrogel spacer injection between the head of the pancreas and duodenum, according to an embodiment herein;

FIG. 5F is an exemplary image of a gross histologic specimen after a laparotomy hydrogel spacer injection between the head of the pancreas and duodenum, according to an embodiment herein;

FIG. 5G is an exemplary image of a computed tomography scan before an endoscopically hydrogel spacer injection between the head of the pancreas and duodenum, according to an embodiment herein;

FIG. 5H is an exemplary image of a computed tomography scan after an endoscopically hydrogel spacer injection between the head of the pancreas and duodenum, according to an embodiment herein;

FIG. 5I is an exemplary image of a gross histologic specimen after an endoscopically hydrogel spacer injection between the head of the pancreas and duodenum, according to an embodiment herein;

FIG. 6A is a first exemplary image of a formalin-fixed, paraffin-embedded section after hematoxylin and eosin staining, according to an embodiment herein;

FIG. 6B is a second exemplary image of a formalin-fixed, paraffin-embedded section after hematoxylin and eosin staining, according to an embodiment herein;

FIG. 6C is a first exemplary high magnification image of a formalin-fixed, paraffin-embedded section after hematoxylin and eosin staining, according to an embodiment herein;

FIG. 6D is a third exemplary image of a formalin-fixed, paraffin-embedded section after hematoxylin and eosin staining, according to an embodiment herein;

FIG. 6E is a second exemplary high magnification image of a formalin-fixed, paraffin-embedded section after hematoxylin and eosin staining, according to an embodiment herein;

FIG. 7A is a first exemplary stereotactic body radiation therapy plan before hydrogel spacer placement, according to an embodiment herein;

FIG. 7B is a first exemplary stereotactic body radiation therapy plan after hydrogel spacer placement, according to an embodiment herein;

FIG. 7C is a second exemplary stereotactic body radiation therapy plan before hydrogel spacer placement, according to an embodiment herein;

FIG. 7D is a second exemplary stereotactic body radiation therapy plan after hydrogel spacer placement, according to an embodiment herein;

FIG. 8A is an exemplary baseline image of a computed tomography scan and stereotactic body radiation therapy plan of the duodenum, according to an embodiment herein;

FIG. 8B is an exemplary image of a computed tomography scan and stereotactic body radiation therapy plan of the duodenum with a 2 mm spacing, according to an embodiment herein;

FIG. 8C is an exemplary image of a computed tomography scan and stereotactic body radiation therapy plan of the duodenum with a 3 mm spacing, according to an embodiment herein;

FIG. 8D is an exemplary image of a computed tomography scan and stereotactic body radiation therapy plan of the duodenum with a 5 mm spacing, according to an embodiment herein;

FIG. 8E is an exemplary image of a computed tomography scan and stereotactic body radiation therapy plan of the duodenum with a 8 mm spacing, according to an embodiment herein;

FIG. 8F is an exemplary image of a computed tomography scan and stereotactic body radiation therapy plan of the duodenum with a 15 mm spacing, according to an embodiment herein;

FIG. 9 shows an MRI scan of bladder markings, a CT scan of liver markings, an MRI of liver markings and an MRI of cervical markings, according to an embodiment herein;

FIG. 10A shows an MRI scan of a submandibular tumor before treatment, according to an embodiment herein;

FIG. 10B shows an MRI scan of a submandibular tumor with distance measurements of about 1 cm, according to an embodiment herein;

FIG. 10C shows an MRI 6 months after the tumor was removed, according to an embodiment herein;

FIG. 11A shows an image of an applicator needle inserted from the left side of the neck, according to an embodiment herein;

FIG. 11B shows an image of the location of a 20 Gy single dose of radiation, according to an embodiment herein;

FIG. 12 shows an MRI image of a paravertebral dosing approach, according to an embodiment herein;

FIG. 13A shows an illustration of a reconstructed rectum and prostate before a brachytherapy radiation, according to an embodiment herein;

FIG. 13B shows an illustration of a reconstructed rectum and prostate after the initial brachytherapy radiation, according to an embodiment herein;

FIG. 14A shows an illustration radiation levels before a brachytherapy radiation with the rectum and prostate being separated by more than 25 mm, according to an embodiment herein;

FIG. 14B shows an illustration radiation levels after a brachytherapy radiation with the rectum and prostate being separated by more than 25 mm, according to an embodiment herein;

FIG. 14C shows an MRI scan of the prostrate and the rectum four hours after injection, according to an embodiment herein;

FIG. 15A shows an X-ray computed tomography image before radiotherapy, according to an embodiment herein;

FIG. 15B shows an X-ray computed tomography image before and external beam radiotherapy treatment plan radiotherapy, according to an embodiment herein;

FIG. 16A shows an ultrasound image and power Doppler image showing the planned route for needle insertion, according to an embodiment herein;

FIG. 16B shows an ultrasound image and power Doppler image showing Brachytherapy dose distribution and inserted brachytherapy needle, according to an embodiment herein;

FIG. 16C shows an ultrasound image after the skin is raised 7 mm, according to an embodiment herein;

FIG. 17A shows an X-ray computed tomography and brachytherapy dose distribution, according to an embodiment herein;

FIG. 17B shows an X-ray computed tomography and images of the second lesion according to an embodiment herein; and

FIG. 17C shows an X-ray computed tomography with no tumor recurrence at 1 year after treatment, according to an embodiment herein.

FIG. 18A shows the initial release of a visualization additive from a viscoelastic medium.

FIG. 18B shows after 7 and 24 hour release analysis of a visualization additive from a viscoelastic medium.

FIG. 19A shows a CT scan of fat injected with 3 ml of viscoelastic gel comprising 90 mg of gold particles.

FIG. 19B shows a CT scan of muscle injected with 3 ml of viscoelastic gel comprising 45 mg of gold particles.

FIG. 19C shows a CT scan of muscle injected with 3 ml of viscoelastic gel comprising 45 mg of gold particles.

FIG. 19D shows a CT scan of muscle injected with 3 ml of viscoelastic gel comprising 90 mg of gold particles.

FIG. 20A shows a CBCT scan of fat injected with 3 ml of viscoelastic gel comprising 90 mg of gold particles.

FIG. 20B shows a CBCT scan of muscle injected with 3 ml of viscoelastic gel comprising 45 mg of gold particles.

FIG. 20C shows a CBCT scan of muscle injected with 3 ml of viscoelastic gel comprising 45 mg of gold particles.

FIG. 20D shows a CBCT scan of muscle injected with 3 ml of viscoelastic gel comprising 90 mg of gold particles.

FIG. 21 shows the location of viscoelastic gel within tissue over a period of time.

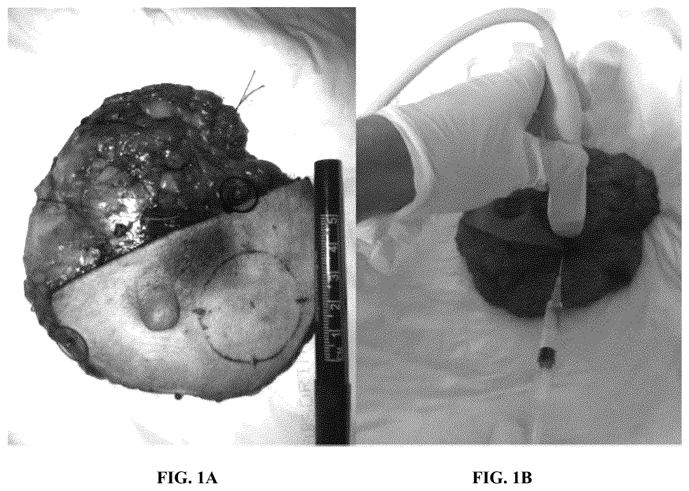

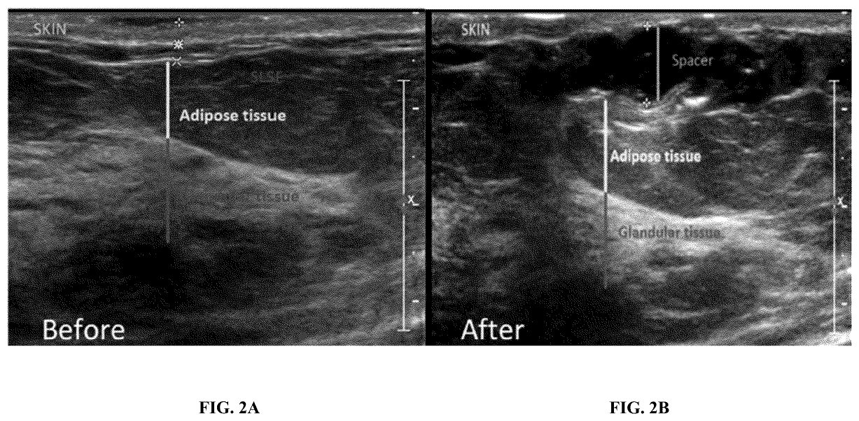

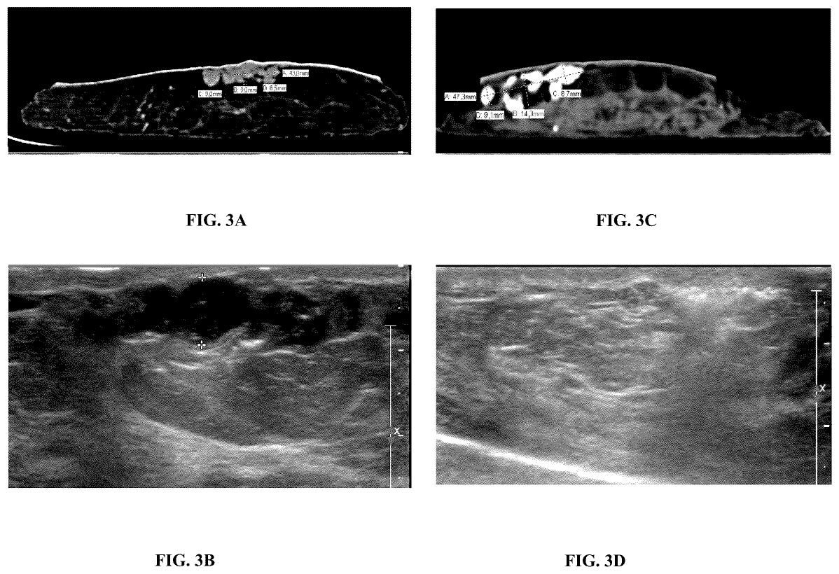

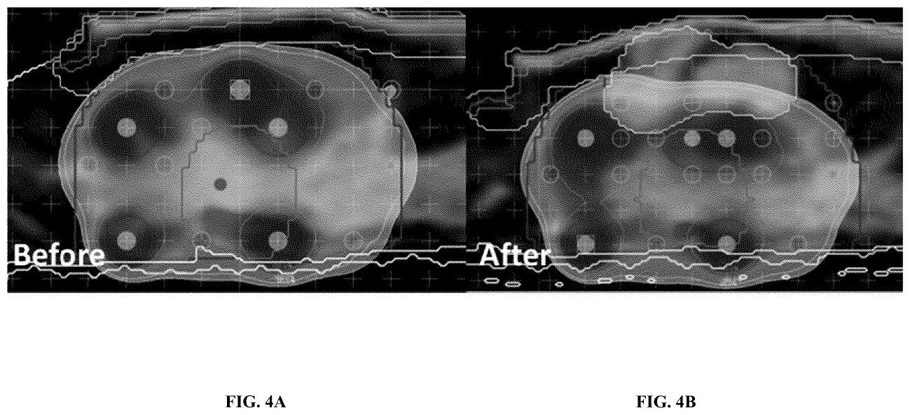

### DETAILED DESCRIPTION

Provided herein are methods for decreasing the toxicity of advanced ablative cancer therapies on neighboring organs. The methods herein provide spacing between single, or multiple, tumor cites and immediate healthy organs while maintaining or increasing patient quality of life. Such toxicity isolation can be performed by inserting a spacer around the one or more tumor cites.

Also provided herein are compositions and methods to be used in conjunction with the spacers described herein to enhance the visual properties of the spacers described herein across various imaging modalities (e.g. x-ray, ultrasound, CT, etc.). In addition to assisting practitioners visualize some, or all, of the interior region of a spacer injected into a patient, the compositions and methods described herein enable practitioners to visualize the contours of a spacer injected into a patient under various imaging modalities.

### Subcutaneous Spacer Materials

The subcutaneous spacer materials herein are configured to form a cavity adjacent to prevent radiation or toxicity damage to organs proximal to or in contact with a treatment organ. Subcutaneous spacer materials may be injected into a tissue site, which may be a discrete location on a single piece of tissue. Tissue sites may include tissues of the subject's breast, head and neck, cervix, vagina, base of spine, skin, pancreas, liver, lungs, rectum, or another tissue site where subcutaneous spacer materials are injected. The subcutaneous spacer materials herein may comprise a viscoelastic media comprising hyaluronic acid particles. The particle size and concentration of the hyaluronic acid within the spacer material can be tuned to exhibit a hardness, density, or both to enable consistent and uniform injection and cavity formation. In some embodiments, the viscoelastic media further comprises visual additive particles. The type of visual additive particles (e.g., precious metal particles) may increase effectiveness as a visual additive by increasing visibility and blocking radiation, and may further prevent travel to and accumulation of at least a subset of the precious metal particles in organs that are not at or surrounding the injection site. In some embodiments, the prevention of travel to and accumulation of at least a subset of the visual additive particles in organs that are not at or surrounding the injection site may be associated with the size of the particles of the subset of the visual additive particles.

In some embodiments, the implant comprises particles of one or more viscoelastic media dispersed in a physiological salt buffer, a suitable physiological salt solvent, or both. In some embodiments, the implant further comprises other additives, such as local anesthetics, anti-inflammatory drugs, antibiotics and supportive medications (e.g. bone growth factors or cells). In some embodiments, there may also be included a viscoelastic medium which may be formed of same material as the particles or a different material than the particles. In some embodiments, the viscoelastic medium is not present as particles.

Viscoelastic media according to embodiments can herein include gels, dispersions, solutions, suspensions, slurries and mixtures thereof. In some embodiments, the medium is present as a dispersion of gel or gel-like particles. The viscoelastic medium provided herein can be more resistant to biodegradation in vivo than natural hyaluronic acid. The prolonged presence of the stable viscoelastic substance is advantageous for the patient, since the time between treatments is increased. The viscoelastic media herein can be biocompatible, sterile, and present as particles. The viscoelastic media may further comprise visual additive particles.

Advantageously, the viscoelastic media herein are stable within, but can be impermanent under, physiological conditions. In some embodiments, about 70% to about 90%, of the viscoelastic medium remains for at least two weeks in vivo. In some embodiments, at least 70%, of the viscoelastic medium remains for at about two weeks and two years in vivo. In some embodiments, at least 90%, of the viscoelastic medium remains for at about two weeks and two years in vivo. The viscoelastic medium can degrade automatically after five years or more in vivo. In some embodiments, if visual additive particles are added to the viscoelastic media, about 50% to about 90% of the visual additive particles of the viscoelastic medium remains for at least nine months in vivo. In some embodiments, at least 50% of the visual additive particles remain for about nine months and two years in vivo. In embodiments, at least 90% of the visual additive particles remain for about nine months and two years in vivo. In some embodiments, at least 10% of the visual additive particles remain indefinitely in vivo.

Viscoelastic media include, without being limited thereto, polysaccharides and derivatives thereof. Suitable viscoelastic media include stabilized starch and derivatives thereof. Suitable viscoelastic media can also be selected from stabilized glycosaminoglycans and derivatives thereof, such as stabilized hyaluronic acid, stabilized chondroitin sulfate, stabilized heparin, and derivatives thereof Δn example of a viscoelastic medium is non-animal stabilized hyaluronic acid (“NASHA”). NASHA is produced from a non-animal source (bacteria). The residence time of the viscoelastic medium is dependent on the size of particles of the viscoelastic medium.

In some embodiments, particles of the viscoelastic medium have a specifically tuned size. The size of the particles can be achieved by producing a gel made of the viscoelastic medium at a desired concentration, and subjecting the gel to a physical disruption. The physical disruption can comprise: mincing, mashing filtering, or any combination thereof.

The resulting gel particles can be dispersed in a physiological salt solution, resulting in a gel dispersion or slurry with particles of desired size. Particle size may be determined in any suitable way, such as by laser diffraction, microscopy, or filtration, etc. In some embodiments, the specific shape of the gel particles is not critical. The size of a spherical particle may be defined by its diameter. The size may be measured as an average size, a median size, a maximum size, or a minimum size.

In some embodiments, the viscoelastic media gel particles have a size in the range of from 1 to 2.5 mm, such as from 1.5 to 2 mm, in the presence of a physiological salt solution. In some embodiments, the viscoelastic media particles have a size in the range of from 2.5 to 5 mm, such as from 3 to 4 mm, in the presence of a physiological salt solution. At least 50% (v/v) of the particles can have a size of at least about 1 mm. At least 50% (v/v) of the viscoelastic media particles can have a size of about 1-5 mm in the presence of a physiological salt solution. In some embodiments, more than 70% (v/v) of the viscoelastic media particles are within the given size limits under physiological conditions. In some embodiments, more than 90% (v/v) of the viscoelastic media particles are within the given size limits under physiological conditions. Administration of the implant employing the method according to an embodiment herein prevents or diminishes migration and/or displacement of the implant, which comprises or consists of the 1-5 mm large particles under physiological conditions. Large particles can exhibit less in vitro migration and can be more easily removed. In some embodiments, the viscoelastic medium is present as particles of a size smaller than 0.1 mm.

In some embodiments, the viscoelastic media gel particles have a size of about 0.05 mm to about 0.1 mm. n some embodiments, the particles have a size of about 0.05 mm to about 0.06 mm, about 0.05 mm to about 0.07 mm, about 0.05 mm to about 0.08 mm, about 0.05 mm to about 0.09 mm, about 0.05 mm to about 0.1 mm, about 0.06 mm to about 0.07 mm, about 0.06 mm to about 0.08 mm, about 0.06 mm to about 0.09 mm, about 0.06 mm to about 0.1 mm, about 0.07 mm to about 0.08 mm, about 0.07 mm to about 0.09 mm, about 0.07 mm to about 0.1 mm, about 0.08 mm to about 0.09 mm, about 0.08 mm to about 0.1 mm, or about 0.09 mm to about 0.1 mm. n some embodiments, the particles have a size of about 0.05 mm, about 0.06 mm, about 0.07 mm, about 0.08 mm, about 0.09 mm, or about 0.1 mm. n some embodiments, the particles have a size of at least about 0.05 mm, about 0.06 mm, about 0.07 mm, about 0.08 mm, or about 0.09 mm. n some embodiments, the particles have a size of at most about 0.06 mm, about 0.07 mm, about 0.08 mm, about 0.09 mm, or about 0.1 mm.

In some embodiments the viscoelastic media gel particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

Suitable viscoelastic media also include stabilized dextran and derivatives thereof, such as dextranomer (Dx). Dx is a large molecule consisting of many cross-linked dextran polymers. The molecular structure of dextran comprises glucose units linked by linear alpha-1, 6-glycosidic bonds with a low degree of small branching. It forms a gel when water is added. For most medical purposes dextran polymers with molecular weights of 70 kDa and 40 kDa are used. When synthesizing Dx these polymers are linked together by adding a cross-linking agent. The manufacturing process causes the dextran polymers to bind together and become cross-linked into small beads known as microspheres. The degree of cross-linkage affects the properties of the Dx, as does the size of the individual microspheres. Dx microspheres can be made in a variety of sizes, and those used in Q-Med's products range between 80 and 250 μm.

In some embodiments, the DX has a molecular weight of about 40 kDa to about 70 kDa. In some embodiments, the Dx has a molecular weight of at most about 70 kDa. In some embodiments, the Dx has a molecular weight of at least about 40 kDa.

In some embodiments, the viscoelastic medium is cross-linked hyaluronic acid, or a derivatives thereof.

One type of suitable cross-linked hyaluronic acid is obtainable by cross-linking of hyaluronic acid. The viscoelastic medium may also be a combination of two or more of the suitable viscoelastic media listed herein or otherwise known to the art. The viscoelastic medium may be of non-animal origin.

In some embodiments, the viscoelastic medium comprises a hydrogel. In some embodiments, the hydrogel is formed from natural, synthetic, or biosynthetic polymers. In some embodiments, the natural polymer comprises glycosminogly cans, polysaccharides, proteins, or any combination thereof. In some embodiments, the glycosaminoglycan is dermatan sulfate, hyaluronic acid, chondroitin sulfate, chitin, heparin, keratan sulfate, keratosulfate, or any combination thereof. In some embodiments, the hydrogel comprises an acidic carboxy polymer, an acrylic acid-based polymer, a polyacrylamide, a starch graft copolymer, an acrylate polymer, or any combination thereof. In some embodiments, the hydrogel comprises allylpentaerythritol, polyacrylic acid, ester cross-linked polyglucan, or any combination thereof.

In some embodiments, the viscoelastic media is hydrophilic.

In some embodiments, the viscoelastic medium comprises a combination of the disclosed compounds. In one example the viscoelastic media comprises hyaluronic acid and Dx. In another example the viscoelastic media comprises NASHA/Dx gel. In some embodiments, the NASHA/Dx gel has a sufficiently low viscosity such that it can be injected through a syringe by finger pressure alone. In some embodiments, the NASHA/Dx gel has a sufficiently high viscosity to avoid leakage from the injection site. In some embodiments, the NASHA/Dx gel has a long degradation time which enables and stabilizes the natural formation of connective tissue at the site of the implant. In some embodiments, the viscoelastic medium further comprises carbon-coated zirconium beads, calcium hydroxylapatite, or both.

The size of the gel particles can depend upon the ionic strength of the buffer, the solution, carrier, or any combination thereof that is included in and/or surrounding the gel particles. As such, given particle sizes can assume physiological conditions, particularly isotonic conditions. In some embodiments, the gel particles contain and are dispersed in a physiological salt solution. In some embodiments, the gel particles are temporarily brought to different sizes by subjecting the gel particles to a solution of another tonicity. Particle sizes within the given ranges under physiological conditions when implanted subepidermally in the body or when subjected to a physiological, or isotonic, salt solution (i.e. a solution with the same tonicity as the relevant biological fluids, such as an isoosmotic with serum).

In some embodiments, the gel particles have a specific tuned size. The size of the gel particles can be achieved by producing a gel made of a viscoelastic medium at a desired concentration, and subjecting the gel to a physical disruption. The physical disruption can comprise: mincing, mashing filtering, or any combination thereof. The resulting gel particles can be dispersed in a physiological salt solution, resulting in a gel dispersion or slurry with particles of desired size. Particle size may be determined in any suitable way, such as by laser diffraction, microscopy, or filtration, etc. In some embodiments, the specific shape of the gel particles is not critical. The size of a spherical particle may be defined as its diameter. The size may be measured as an average size, a median size, a maximum size, or a minimum size.

In some embodiments, the gel particles have a size in the range of from 1 to 2.5 mm, such as from 1.5 to 2 mm, in the presence of a physiological salt solution. In some embodiments, the gel particles have a size in the range of from 2.5 to 5 mm, such as from 3 to 4 mm, in the presence of a physiological salt solution. At least 50% (v/v) of the particles can have a size of at least about 1 mm. At least 50% (v/v) of the particles can have a size of about 1-5 mm in the presence of a physiological salt solution. In some embodiments, more than 70% (v/v) of the gel particles are within the given size limits under physiological conditions.

In some embodiments, more than 90% (v/v) of the particles are within the given size limits under physiological conditions. Administration of the implant employing the method according to an embodiment herein prevents or diminishes migration and/or displacement of the implant, which comprises or consists of the 1-5 mm large gel particles under physiological conditions. Large gel particles can exhibit less in vitro migration and can be more easily removed. In some embodiments, the viscoelastic medium is not present as particles of a size smaller than 0.1 mm. In some embodiments, the Dx is composed of microspheres. In some embodiments, the microspheres have a diameter of about 80 μm to about 250 μm. In some embodiments, the microspheres have a diameter of at least about 80 μm. In some embodiments, the microspheres have a diameter of at most about 250 μm. In some embodiments, the Dx is composed of microspheres. In some embodiments, the microspheres have a diameter of about 80 μm to about 250 μm. In some embodiments, the microspheres have a diameter of at least about 80 μm. In some embodiments, the microspheres have a diameter of at most about 250 μm

In some embodiments, the particles have a specific tuned density, hardness or both. The gel particle density can be regulated by adjusting the concentration of the viscoelastic medium, the amount and type of cross-linking agent, or both. Harder particles can be achieved by increased concentration of the viscoelastic medium in the gel. Harder particles can be less viscoelastic and exhibit a longer half-life in vivo than softer particles. The particles herein should retain enough viscoelastic properties that they can be safely injected. In some embodiments, the implant comprises both soft gel particles and harder gel particles. The soft and hard gel particles may be made of the same or different viscoelastic media. The resulting mixture of gel particles combines desirable properties of softness/hardness for use in radiative protection and long durability in vivo.

### Methods of Forming a Spacer Material

The subcutaneous spacer materials herein are configured to form a cavity adjacent to prevent radiation or toxicity damage to organs proximal to or in contact with a treatment organ. The subcutaneous spacer materials herein may comprise a viscoelastic media comprising hyaluronic acid particles. The particle size and concentration of the hyaluronic acid within the spacer material can be tuned to exhibit a hardness, density, or both to enable consistent and uniform injection and cavity formation.

Provided herein are methods of forming a spacer material comprising forming an aqueous solution comprising: a water soluble cross-linkable polysaccharide; initiating a cross-linking of the polysaccharide in the presence of a polyfunctional cross-linking agent; sterically hindering the cross-linking reaction from terminating before gelation occurs to generate activated polysaccharide; mechanically mixing about 45 mg to about 90 mg of visual additive particles and reintroducing the sterically unhindered conditions for the activated polysaccharide to continue the cross-linking thereof up to a viscoelastic gel. In some embodiments, the individual visual additive particles have diameters of about 80 micrometers to about 120 micrometers. In some embodiments, the visual additive particles are gold particles. In some embodiments, the initial cross-linking reaction in the presence of a polyfunctional cross-linking agent can be performed at varying pH values, primarily depending on whether ether or ester reactions should be promoted.

The cross-linking agent can be any previously known cross-linking agent useful in connection with polysaccharides that are biocompatible. However, the cross-linking agent is comprises: aldehydes, epoxides, polyaziridyl compounds, glycidyl ethers, divinylsulfones, or any combination thereof. Glycidyl ethers represent a group, of which 1,4-butanediol diglycidyl ether can be advantageous. In some embodiments, the spacer material comprises a hydrogel comprising a glycosaminoglycan that is extracted from a natural source that is purified and derivatized. In some embodiments, the glycosaminoglycan is synthetically produced or synthesized by modified microorganisms such as bacteria. In some embodiments, the glycosaminoglycan is modified synthetically from a naturally soluble state to a partially soluble or water swellable or hydrogel state.

A suitable way of obtaining a desired particle size involves producing a gel made of cross-linked hyaluronic acid at a desired concentration and subjecting the gel to physical disruption, such as mincing, mashing or allowing the gel to pass through a filter with suitable particle size. The resulting gel particles are dispersed in a physiological salt solution, resulting in a gel dispersion or slurry with particles of desired size. The size of the particles can be achieved by producing a gel made of a viscoelastic medium at a desired concentration, and subjecting the gel to a physical disruption. The physical disruption can comprise: mincing, mashing filtering, or any combination thereof. The resulting gel particles can be dispersed in a physiological salt solution, resulting in a gel dispersion or slurry with particles of desired size.

In some embodiments, the gel particles have a specific tuned density, hardness or both. The gel particle density can be regulated by adjusting the concentration of the viscoelastic medium, the amount and type of cross-linking agent, or both. Harder particles can be achieved by increased concentration of the viscoelastic medium in the gel. By varying the hyaluronic acid concentrations to, for example, 20, 25, 40, 50 and 100 mg/ml gel particles of varying hardness can be obtained. Harder particles can be less viscoelastic and exhibit a longer half-life in vivo than softer particles. The particles herein should retain enough viscoelastic properties that they can be safely injected.

In some embodiments, the implant comprises both soft gel particles and harder gel particles. The soft and hard gel particles may be made of the same or different viscoelastic media. The resulting mixture of gel particles combines desirable properties of softness/hardness for use in radiative protection and long durability in vivo. In one embodiment the soft gel particles comprise 15-22 mg/ml of the cross-linked hyaluronic acid, and the hard gel particles comprise 22-30 mg/ml of the cross-linked hyaluronic acid.

When the injectable medium is a hyaluronic acid medium, the hyaluronic acid concentration can be at least about 5 mg/ml. In some embodiments, the hyaluronic acid concentration is about 5 mg/ml to about 100 mg/ml. In some embodiments, the hyaluronic acid concentration is about 10 to about 50 mg/ml. In some embodiments, the hyaluronic acid concentration is about 20 mg/ml. The cross-linked hyaluronic acid can be present as particles or beads of any form.

In some embodiments, the method further comprises adding an image enhancement agent described herein to the spacer material. In some embodiments, the method further comprises mixing in an image enhancement agent described herein to the spacer material.

### Methods of Injecting a Spacer Material

The subcutaneous spacer materials herein are configured to form a cavity adjacent to prevent radiation or toxicity damage to organs proximal to or in contact with a treatment organ. The subcutaneous spacer materials herein may comprise a viscoelastic media comprising hyaluronic acid particles. The particle size and concentration of the hyaluronic acid within the spacer material can be tuned to exhibit a hardness, density, or both to enable consistent and uniform injection and cavity formation. Further, a specific needle size can be used to deliver the spacer material to its intended in vivo location based on the particle size, hardness, density, and concentration of the hyaluronic acid.

Provided herein is a method of injecting a spacing material. The spacing material can comprise a viscoelastic medium for therapeutic radiative protection in a mammal, including man. The spacing material can suitable for subepidermal administration at a site in said mammal where therapeutic soft tissue protection is required from radiation or other toxic sources. In particular, the particles are suitable for administration to tissues covered by publicly hidden skin or darker skin, such as rectal tissue, as the particles are configured to provide maximum contrast and visibility. The particles herein are suitable for administration into deep subcutaneous or to submuscular/supraperiostal tissue, optionally in more than one layer. Deep subcutaneous or submuscular/supraperiostal administration can further prevent or diminished migration of the particles away from the desired site.

The spacing material can be administered by injection under the epidermis in any suitable way. By way of example, a dermal incision can be made with a scalpel or a sharp injection needle to facilitate transdermal insertion of a larger cannula for administration of the implant at the desired site.

The implant, consisting of particles of a viscoelastic medium and optionally other suitable ingredients, may be administered as a single aliquot or as layers of multiple aliquots. Optionally, the viscoelastic medium may be replaced, refilled or replenished by a subsequent injection of the same or another viscoelastic medium. The injected volume is determined by the size of the desired cavity.

In some embodiments, a volume of the spacer material that is injected is about 1 ml to about 500 ml. In some embodiments, a volume of the spacer material that is injected is about 1 ml to about 5 ml, about 1 ml to about 10 ml, about 1 ml to about 25 ml, about 1 ml to about 50 ml, about 1 ml to about 100 ml, about 1 ml to about 150 ml, about 1 ml to about 200 ml, about 1 ml to about 250 ml, about 1 ml to about 300 ml, about 1 ml to about 400 ml, about 1 ml to about 500 ml, about 5 ml to about 10 ml, about 5 ml to about 25 ml, about 5 ml to about 50 ml, about 5 ml to about 100 ml, about 5 ml to about 150 ml, about 5 ml to about 200 ml, about 5 ml to about 250 ml, about 5 ml to about 300 ml, about 5 ml to about 400 ml, about 5 ml to about 500 ml, about 10 ml to about 25 ml, about 10 ml to about 50 ml, about 10 ml to about 100 ml, about 10 ml to about 150 ml, about 10 ml to about 200 ml, about 10 ml to about 250 ml, about 10 ml to about 300 ml, about 10 ml to about 400 ml, about 10 ml to about 500 ml, about 25 ml to about 50 ml, about 25 ml to about 100 ml, about 25 ml to about 150 ml, about 25 ml to about 200 ml, about 25 ml to about 250 ml, about 25 ml to about 300 ml, about 25 ml to about 400 ml, about 25 ml to about 500 ml, about 50 ml to about 100 ml, about 50 ml to about 150 ml, about 50 ml to about 200 ml, about 50 ml to about 250 ml, about 50 ml to about 300 ml, about 50 ml to about 400 ml, about 50 ml to about 500 ml, about 100 ml to about 150 ml, about 100 ml to about 200 ml, about 100 ml to about 250 ml, about 100 ml to about 300 ml, about 100 ml to about 400 ml, about 100 ml to about 500 ml, about 150 ml to about 200 ml, about 150 ml to about 250 ml, about 150 ml to about 300 ml, about 150 ml to about 400 ml, about 150 ml to about 500 ml, about 200 ml to about 250 ml, about 200 ml to about 300 ml, about 200 ml to about 400 ml, about 200 ml to about 500 ml, about 250 ml to about 300 ml, about 250 ml to about 400 ml, about 250 ml to about 500 ml, about 300 ml to about 400 ml, about 300 ml to about 500 ml, or about 400 ml to about 500 ml. In some embodiments, a volume of the spacer material that is injected is about 1 ml, about 5 ml, about 10 ml, about 25 ml, about 50 ml, about 100 ml, about 150 ml, about 200 ml, about 250 ml, about 300 ml, about 400 ml, or about 500 ml. In some embodiments, a volume of the spacer material that is injected is at least about 1 ml, about 5 ml, about 10 ml, about 25 ml, about 50 ml, about 100 ml, about 150 ml, about 200 ml, about 250 ml, about 300 ml, or about 400 ml. In some embodiments, a volume of the spacer material that is injected is at most about 5 ml, about 10 ml, about 25 ml, about 50 ml, about 100 ml, about 150 ml, about 200 ml, about 250 ml, about 300 ml, about 400 ml, or about 500 ml.

Administration may be performed in any suitable way, such as via injection from standard cannula and needles of appropriate sizes. The administration is performed where the radiative protection is desired, such as the chin, cheeks or elsewhere in the face or body.

The spacing material herein is injectable through standard needles used in medicine, such as 20 gauge or larger needles. Alternatively the spacing material comprising hyaluronic acid can be injected using any of the following sized needles:

| | | Gauge | | | | | | | | | | |
|---|---|---|---|---|---|---|---|---|---|---|---|---|
| | | 15 | 16 | 17 | 18 | 19 | 20 | 21 | 22 | 23 | 24 | 25 |
| Needle | 0.5 | A1 | A2 | A3 | A4 | A5 | A6 | A7 | A8 | A9 | A10 | A11 |
| [symbol] | 0.375 | B1 | B2 | B3 | B4 | B5 | B6 | B7 | B8 | B9 | B10 | B11 |
| | .75 | C1 | C2 | C3 | C4 | C5 | C6 | C7 | C8 | C9 | C10 | C11 |
| | 1 | D1 | D2 | D3 | D4 | D5 | D6 | D7 | D8 | D9 | D10 | D11 |
| | 1.25 | E1 | E2 | E3 | E4 | E5 | E6 | E7 | E8 | E9 | E10 | E11 |
| | 1.5 | F1 | F2 | F3 | F4 | F5 | F6 | F7 | F8 | F9 | F10 | F11 |
| | 2 | G1 | G2 | G3 | G4 | G5 | G6 | G7 | G8 | G9 | G10 | G11 |

[symbol]

indicates data missing or illegible when filed

In some embodiments, an interior surface of the needle comprises a protrusion, a mesh, a constriction, or any combination thereof. In some embodiments, injecting the spacing material past the protrusion, the mesh, the constriction, or any combination thereof produces a gas bubble in the spacing material. In some embodiments, the gas bubble is a microbubble.

In some embodiments, the mesh has a mesh spacing of about 20 μm to about 300 μm. In some embodiments, a size of the mesh spacing determines a size of the microbubbles produced thereby.

### Visual Additives

CT imaging enhancement development can be used to leverage the stability and visibility of the viscoelastic media when a visual additive is added to the viscoelastic media.

For example, the addition of a precious metal (e.g., gold) or other visual additive (e.g., iodine, gadolinium, iron, barium, calcium, or magnesium) may increase the contrast and reflectiveness of the viscoelastic media. Thus, the addition of the precious metal allows for increased effectiveness of the viscoelastic media with a visual additive by both increasing visibility and decreasing the negative effects of radiation.

In some embodiments, the effectiveness of the viscoelastic media with a visual additive may be measured. In those embodiments, a measurement method may include contrast to noise ratio (“CNR”) of an associated scan. Contrast may be measured based on ratio of the luminance between the injected tissues and the luminance of the surrounding tissue in the associated scan. In some embodiments, the contrast is measured as the difference in grayscale signal of the tissue of the injection site and the grayscale signal of the tissue surrounding the injection site. Noise may be measured based on the signal to noise ratio of the associated scan, where the signal is the measure of true signal and noise is random quantum mottle.

In particular, in some embodiments, the viscoelastic media with a visual additive (e.g., gold particles) may provide enhanced features as compared to other viscoelastic media. For example, viscoelastic media with visual additives may have improved visibility as well as longer residence time in the body due to a slower rate of resorption. For example, the viscoelastic media with a visual additive may take up to about 1 year to about 3 years to fully resorb into tissue. In another example, the viscoelastic media with a visual additive may remain indefinitely in the tissue without being resorbed. Further, viscoelastic media with visual additives may exhibit an increased ability to hold its shape once injected. For example, the viscoelastic media with a visual additive with a certain shape may be compressed for a period of time so that it is no longer in that certain shape, and once the compression ends, the viscoelastic media may return to the certain shape.

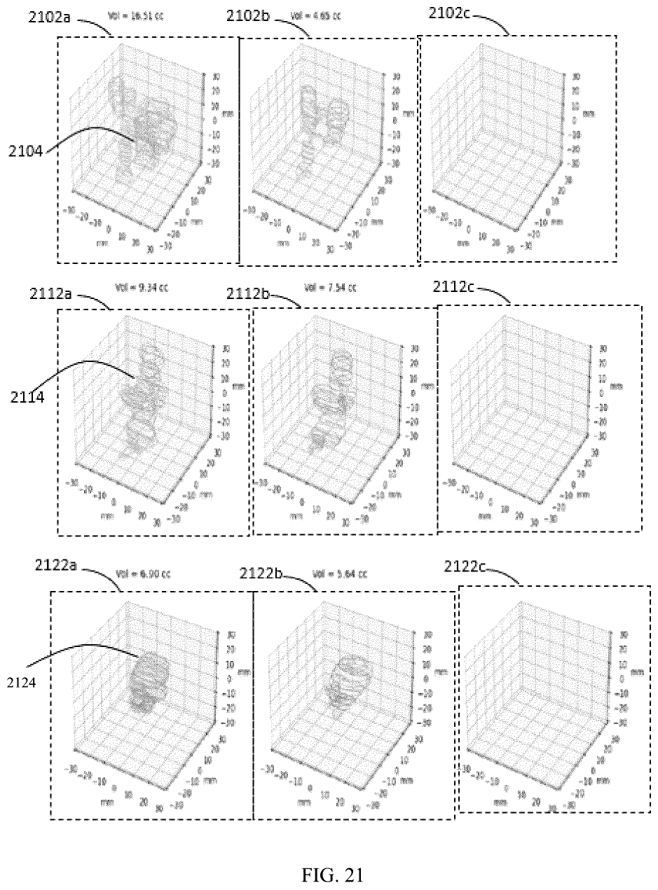

FIG. 21 depicts amounts of viscoelastic media with a visual additive in a compartment of a body over time. Three exemplary viscoelastic media 2104, 2114, and 2124 were placed in respective compartments. The shape and volume of viscoelastic media 2104 is shown in graphs 2102a, 2102b, and 2102c. Graph 2102a shows the shape and volume within seven days of being injected into the respective compartment, where the volume of viscoelastic media 2104 is 16.51 cubic centimeters. Graph 2102b shows the shape and volume at about three months after being injected into the respective compartment, where the volume of viscoelastic media 2104 is 4.65 cubic centimeters. Graph 2102c shows the shape and volume at about three months after being injected into the respective compartment, where the viscoelastic media 2104 is no longer in the compartment. Graph 2112a shows the shape and volume within seven days of being injected into the respective compartment, where the volume of viscoelastic media 2114 is 9.34 cubic centimeters. Graph 2112b shows the shape and volume at about three months after being injected into the respective compartment, where the volume of viscoelastic media 2114 is 7.54 cubic centimeters. Graph 2112c shows the shape and volume at about three months after being injected into the respective compartment, where the viscoelastic media 2114 is no longer in the compartment.

In some embodiments, the visual additive particles have a size of about 15 micrometers (μm) to about 215 μm. In some embodiments, the visual additive particles have a size of about 15 μm to about 35 μm, about 15 μm to about 55 μm, about 15 μm to about 75 μm, about 15 μm to about 95 μm, about 15 μm to about 115 μm, about 15 μm to about 135 μm, about 15 μm to about 155 μm, about 15 μm to about 175 μm, about 15 μm to about 195 μm, about 15 μm to about 215 μm, about 35 μm to about 55 μm, about 35 μm to about 75 μm, about 35 μm to about 95 μm, about 35 μm to about 115 μm, about 35 μm to about 135 μm, about 35 μm to about 155 μm, about 35 μm to about 175 μm, about 35 μm to about 195 μm, about 35 μm to about 215 μm, about 55 μm to about 75 μm, about 55 μm to about 95 μm, about 55 μm to about 115 μm, about 55 μm to about 135 μm, about 55 μm to about 155 μm, about 55 μm to about 175 μm, about 55 μm to about 195 μm, about 55 μm to about 215 μm, about 75 μm to about 95 μm, about 75 μm to about 115 μm, about 75 μm to about 135 μm, about 75 μm to about 155 μm, about 75 μm to about 175 μm, about 75 μm to about 195 μm, about 75 μm to about 215 μm, about 95 μm to about 115 μm, about 95 μm to about 135 μm, about 95 μm to about 155 μm, about 95 μm to about 175 μm, about 95 μm to about 195 μm, about 95 μm to about 215 μm, about 115 μm to about 135 μm, about 115 μm to about 155 μm, about 115 μm to about 175 μm, about 115 μm to about 195 μm, about 115 μm to about 215 μm, about 135 μm to about 155 μm, about 135 μm to about 175 μm, about 135 μm to about 195 μm, about 135 μm to about 215 μm, about 155 μm to about 175 μm, about 155 μm to about 195 μm, about 155 μm to about 215 μm, about 175 μm to about 195 μm, about 175 μm to about 215 μm, or about 195 μm to about 215 μm. In some embodiments, the visual additive particles have a size of about 15 μm, about 35 μm, about 55 μm, about 75 μm, about 95 μm, about 115 μm, about 135 μm, about 155 μm, about 175 μm, about 195 μm, or about 215 μm. In some embodiments, the visual additive particles have a size of at least about 15 μm, about 35 μm, about 55 μm, about 75 μm, about 95 μm, about 115 μm, about 135 μm, about 155 μm, about 175 μm, or about 195 μm. In some embodiments, the visual additive particles have a size of at most about 35 μm, about 55 μm, about 75 μm, about 95 μm, about 115 μm, about 135 μm, about 155 μm, about 175 μm, about 195 μm, or about 215 μm.

In some embodiments, the visual additive particles have a size of about 80 μm to about 120 μm. In some embodiments, the visual additive particles have a size of about 80 μm to about 85 μm, about 80 μm to about 90 μm, about 80 μm to about 95 μm, about 80 μm to about 100 μm, about 80 μm to about 105 μm, about 80 μm to about 110 μm, about 80 μm to about 115 μm, about 80 μm to about 120 μm, about 85 μm to about 90 μm, about 85 μm to about 95 μm, about 85 μm to about 100 μm, about 85 μm to about 105 μm, about 85 μm to about 110 μm, about 85 μm to about 115 μm, about 85 μm to about 120 μm, about 90 μm to about 95 μm, about 90 μm to about 100 μm, about 90 μm to about 105 μm, about 90 μm to about 110 μm, about 90 μm to about 115 μm, about 90 μm to about 120 μm, about 95 μm to about 100 μm, about 95 μm to about 105 μm, about 95 μm to about 110 μm, about 95 μm to about 115 μm, about 95 μm to about 120 μm, about 100 μm to about 105 μm, about 100 μm to about 110 μm, about 100 μm to about 115 μm, about 100 μm to about 120 μm, about 105 μm to about 110 μm, about 105 μm to about 115 μm, about 105 μm to about 120 μm, about 110 μm to about 115 μm, about 110 μm to about 120 μm, or about 115 μm to about 120 μm. In some embodiments, the visual additive particles have a size of about 80 μm, about 85 μm, about 90 μm, about 95 μm, about 100 μm, about 105 μm, about 110 μm, about 115 μm, or about 120 μm. In some embodiments, the visual additive particles have a size of at least about 80 μm, about 85 μm, about 90 μm, about 95 μm, about 100 μm, about 105 μm, about 110 μm, or about 115 μm. In some embodiments, the visual additive particles have a size of at most about 85 μm, about 90 μm, about 95 μm, about 100 μm, about 105 μm, about 110 μm, about 115 μm, or about 120 μm.

In some embodiments, the viscoelastic media with a visual additive may be created using NASHA technology. In those embodiments, the viscoelastic media may be pushed through a mesh. In some embodiments, the mesh is made of metal. In some embodiments, the metal is stainless steel. In some embodiments, pushing the viscoelastic media through the mesh results in viscoelastic particles of a diameter and/or size as described herein. In some embodiments, the viscoelastic particles of a diameter of 0.1 mm to 5.0 mm.

In some embodiments, the viscoelastic media with the visual additive may have an increased CNR in obtained scans (such as CT scans and CBCT scans). In some embodiments, the viscoelastic media with the visual additive may lead to an increased CNR in obtained scans relative to the CNR in obtained scans for viscoelastic media without the visual additive. In some embodiments, the viscoelastic media comprise a plurality of visual additive particles as described herein. In some embodiments, the plurality of visual additives particles are present in the viscoelastic media in an amount that generates an increased CNR in obtained scans relative to the CNR in obtained scans for viscoelastic media without the visual additive. In some embodiments, the CNR may be about 0.1 to about 1. In some embodiments, the CNR may be about 0.1 to about 0.2, about 0.1 to about 0.3, about 0.1 to about 0.4, about 0.1 to about 0.5, about 0.1 to about 0.6, about 0.1 to about 0.7, about 0.1 to about 0.8, about 0.1 to about 0.9, about 0.1 to about 1, about 0.2 to about 0.3, about 0.2 to about 0.4, about 0.2 to about 0.5, about 0.2 to about 0.6, about 0.2 to about 0.7, about 0.2 to about 0.8, about 0.2 to about 0.9, about 0.2 to about 1, about 0.3 to about 0.4, about 0.3 to about 0.5, about 0.3 to about 0.6, about 0.3 to about 0.7, about 0.3 to about 0.8, about 0.3 to about 0.9, about 0.3 to about 1, about 0.4 to about 0.5, about 0.4 to about 0.6, about 0.4 to about 0.7, about 0.4 to about 0.8, about 0.4 to about 0.9, about 0.4 to about 1, about 0.5 to about 0.6, about 0.5 to about 0.7, about 0.5 to about 0.8, about 0.5 to about 0.9, about 0.5 to about 1, about 0.6 to about 0.7, about 0.6 to about 0.8, about 0.6 to about 0.9, about 0.6 to about 1, about 0.7 to about 0.8, about 0.7 to about 0.9, about 0.7 to about 1, about 0.8 to about 0.9, about 0.8 to about 1, or about 0.9 to about 1. In some embodiments, the CNR may be about 0.1, about 0.2, about 0.3, about 0.4, about 0.5, about 0.6, about 0.7, about 0.8, about 0.9, or about 1. In some embodiments, the CNR may be at least about 0.1, about 0.2, about 0.3, about 0.4, about 0.5, about 0.6, about 0.7, about 0.8, or about 0.9. In some embodiments, the CNR may be at most about 0.2, about 0.3, about 0.4, about 0.5, about 0.6, about 0.7, about 0.8, about 0.9, or about 1. In some embodiments, the CNR may be about 1 to about 5.2. In some embodiments, the CNR may be about 1 to about 1.6, about 1 to about 2.2, about 1 to about 2.8, about 1 to about 3.4, about 1 to about 4, about 1 to about 4.6, about 1 to about 5.2, about 1.6 to about 2.2, about 1.6 to about 2.8, about 1.6 to about 3.4, about 1.6 to about 4, about 1.6 to about 4.6, about 1.6 to about 5.2, about 2.2 to about 2.8, about 2.2 to about 3.4, about 2.2 to about 4, about 2.2 to about 4.6, about 2.2 to about 5.2, about 2.8 to about 3.4, about 2.8 to about 4, about 2.8 to about 4.6, about 2.8 to about 5.2, about 3.4 to about 4, about 3.4 to about 4.6, about 3.4 to about 5.2, about 4 to about 4.6, about 4 to about 5.2, or about 4.6 to about 5.2. In some embodiments, the CNR may be about 1, about 1.6, about 2.2, about 2.8, about 3.4, about 4, about 4.6, or about 5.2. In some embodiments, the CNR may be at least about 1, about 1.6, about 2.2, about 2.8, about 3.4, about 4, or about 4.6. In some embodiments, the CNR may be at most about 1.6, about 2.2, about 2.8, about 3.4, about 4, about 4.6, or about 5.2. In some embodiments, the CNR may be at least 5.2, e.g., about 5.2 to about 15. In some embodiments, the CNR may be at least 5.2, e.g., about 5.2 to about 6, about 5.2 to about 6.8, about 5.2 to about 7.6, about 5.2 to about 8.4, about 5.2 to about 9.2, about 5.2 to about 10, about 5.2 to about 11, about 5.2 to about 12, about 5.2 to about 13, about 5.2 to about 14, about 5.2 to about 15, about 6 to about 6.8, about 6 to about 7.6, about 6 to about 8.4, about 6 to about 9.2, about 6 to about 10, about 6 to about 11, about 6 to about 12, about 6 to about 13, about 6 to about 14, about 6 to about 15, about 6.8 to about 7.6, about 6.8 to about 8.4, about 6.8 to about 9.2, about 6.8 to about 10, about 6.8 to about 11, about 6.8 to about 12, about 6.8 to about 13, about 6.8 to about 14, about 6.8 to about 15, about 7.6 to about 8.4, about 7.6 to about 9.2, about 7.6 to about 10, about 7.6 to about 11, about 7.6 to about 12, about 7.6 to about 13, about 7.6 to about 14, about 7.6 to about 15, about 8.4 to about 9.2, about 8.4 to about 10, about 8.4 to about 11, about 8.4 to about 12, about 8.4 to about 13, about 8.4 to about 14, about 8.4 to about 15, about 9.2 to about 10, about 9.2 to about 11, about 9.2 to about 12, about 9.2 to about 13, about 9.2 to about 14, about 9.2 to about 15, about 10 to about 11, about 10 to about 12, about 10 to about 13, about 10 to about 14, about 10 to about 15, about 11 to about 12, about 11 to about 13, about 11 to about 14, about 11 to about 15, about 12 to about 13, about 12 to about 14, about 12 to about 15, about 13 to about 14, about 13 to about 15, or about 14 to about 15. In some embodiments, the CNR may be at least 5.2, e.g., about 5.2, about 6, about 6.8, about 7.6, about 8.4, about 9.2, about 10, about 11, about 12, about 13, about 14, or about 15. In some embodiments, the CNR may be at least 5.2, e.g., at least about 5.2, about 6, about 6.8, about 7.6, about 8.4, about 9.2, about 10, about 11, about 12, about 13, or about 14. In some embodiments, the CNR may be at least 5.2, e.g., at most about 6, about 6.8, about 7.6, about 8.4, about 9.2, about 10, about 11, about 12, about 13, about 14, or about 15. In some embodiments, the CNR may be at least 14.3, e.g., about 14.3 to about 23.1. In some embodiments, the CNR may be at least 14.3, e.g., about 14.3 to about 15.1, about 14.3 to about 15.9, about 14.3 to about 16.7, about 14.3 to about 17.5, about 14.3 to about 18.3, about 14.3 to about 19.1, about 14.3 to about 19.9, about 14.3 to about 20.7, about 14.3 to about 21.5, about 14.3 to about 22.3, about 14.3 to about 23.1, about 15.1 to about 15.9, about 15.1 to about 16.7, about 15.1 to about 17.5, about 15.1 to about 18.3, about 15.1 to about 19.1, about 15.1 to about 19.9, about 15.1 to about 20.7, about 15.1 to about 21.5, about 15.1 to about 22.3, about 15.1 to about 23.1, about 15.9 to about 16.7, about 15.9 to about 17.5, about 15.9 to about 18.3, about 15.9 to about 19.1, about 15.9 to about 19.9, about 15.9 to about 20.7, about 15.9 to about 21.5, about 15.9 to about 22.3, about 15.9 to about 23.1, about 16.7 to about 17.5, about 16.7 to about 18.3, about 16.7 to about 19.1, about 16.7 to about 19.9, about 16.7 to about 20.7, about 16.7 to about 21.5, about 16.7 to about 22.3, about 16.7 to about 23.1, about 17.5 to about 18.3, about 17.5 to about 19.1, about 17.5 to about 19.9, about 17.5 to about 20.7, about 17.5 to about 21.5, about 17.5 to about 22.3, about 17.5 to about 23.1, about 18.3 to about 19.1, about 18.3 to about 19.9, about 18.3 to about 20.7, about 18.3 to about 21.5, about 18.3 to about 22.3, about 18.3 to about 23.1, about 19.1 to about 19.9, about 19.1 to about 20.7, about 19.1 to about 21.5, about 19.1 to about 22.3, about 19.1 to about 23.1, about 19.9 to about 20.7, about 19.9 to about 21.5, about 19.9 to about 22.3, about 19.9 to about 23.1, about 20.7 to about 21.5, about 20.7 to about 22.3, about 20.7 to about 23.1, about 21.5 to about 22.3, about 21.5 to about 23.1, or about 22.3 to about 23.1. In some embodiments, the CNR may be at least 14.3, e.g., about 14.3, about 15.1, about 15.9, about 16.7, about 17.5, about 18.3, about 19.1, about 19.9, about 20.7, about 21.5, about 22.3, or about 23.1. In some embodiments, the CNR may be at least 14.3, e.g., at least about 14.3, about 15.1, about 15.9, about 16.7, about 17.5, about 18.3, about 19.1, about 19.9, about 20.7, about 21.5, or about 22.3. In some embodiments, the CNR may be at least 14.3, e.g., at most about 15.1, about 15.9, about 16.7, about 17.5, about 18.3, about 19.1, about 19.9, about 20.7, about 21.5, about 22.3, or about 23.1. In some embodiments, the CNR may be at least 15.9, e.g., about 16.9 to about 27.9. In some embodiments, the CNR may be at least 15.9, e.g., about 16.9 to about 17.9, about 16.9 to about 18.9, about 16.9 to about 19.9, about 16.9 to about 20.9, about 16.9 to about 21.9, about 16.9 to about 22.9, about 16.9 to about 23.9, about 16.9 to about 24.9, about 16.9 to about 25.9, about 16.9 to about 26.9, about 16.9 to about 27.9, about 17.9 to about 18.9, about 17.9 to about 19.9, about 17.9 to about 20.9, about 17.9 to about 21.9, about 17.9 to about 22.9, about 17.9 to about 23.9, about 17.9 to about 24.9, about 17.9 to about 25.9, about 17.9 to about 26.9, about 17.9 to about 27.9, about 18.9 to about 19.9, about 18.9 to about 20.9, about 18.9 to about 21.9, about 18.9 to about 22.9, about 18.9 to about 23.9, about 18.9 to about 24.9, about 18.9 to about 25.9, about 18.9 to about 26.9, about 18.9 to about 27.9, about 19.9 to about 20.9, about 19.9 to about 21.9, about 19.9 to about 22.9, about 19.9 to about 23.9, about 19.9 to about 24.9, about 19.9 to about 25.9, about 19.9 to about 26.9, about 19.9 to about 27.9, about 20.9 to about 21.9, about 20.9 to about 22.9, about 20.9 to about 23.9, about 20.9 to about 24.9, about 20.9 to about 25.9, about 20.9 to about 26.9, about 20.9 to about 27.9, about 21.9 to about 22.9, about 21.9 to about 23.9, about 21.9 to about 24.9, about 21.9 to about 25.9, about 21.9 to about 26.9, about 21.9 to about 27.9, about 22.9 to about 23.9, about 22.9 to about 24.9, about 22.9 to about 25.9, about 22.9 to about 26.9, about 22.9 to about 27.9, about 23.9 to about 24.9, about 23.9 to about 25.9, about 23.9 to about 26.9, about 23.9 to about 27.9, about 24.9 to about 25.9, about 24.9 to about 26.9, about 24.9 to about 27.9, about 25.9 to about 26.9, about 25.9 to about 27.9, or about 26.9 to about 27.9. In some embodiments, the CNR may be at least 15.9, e.g., about 16.9, about 17.9, about 18.9, about 19.9, about 20.9, about 21.9, about 22.9, about 23.9, about 24.9, about 25.9, about 26.9, or about 27.9. In some embodiments, the CNR may be at least 15.9, e.g., at least about 16.9, about 17.9, about 18.9, about 19.9, about 20.9, about 21.9, about 22.9, about 23.9, about 24.9, about 25.9, or about 26.9. In some embodiments, the CNR may be at least 15.9, e.g., at most about 17.9, about 18.9, about 19.9, about 20.9, about 21.9, about 22.9, about 23.9, about 24.9, about 25.9, about 26.9, or about 27.9. In some embodiments, the CNR may be at least 20.2, e.g., about 20.2 to about 40. In some embodiments, the CNR may be at least 20.2, e.g., about 20.2 to about 21.1, about 20.2 to about 22, about 20.2 to about 24, about 20.2 to about 26, about 20.2 to about 28, about 20.2 to about 30, about 20.2 to about 32, about 20.2 to about 34, about 20.2 to about 36, about 20.2 to about 38, about 20.2 to about 40, about 21.1 to about 22, about 21.1 to about 24, about 21.1 to about 26, about 21.1 to about 28, about 21.1 to about 30, about 21.1 to about 32, about 21.1 to about 34, about 21.1 to about 36, about 21.1 to about 38, about 21.1 to about 40, about 22 to about 24, about 22 to about 26, about 22 to about 28, about 22 to about 30, about 22 to about 32, about 22 to about 34, about 22 to about 36, about 22 to about 38, about 22 to about 40, about 24 to about 26, about 24 to about 28, about 24 to about 30, about 24 to about 32, about 24 to about 34, about 24 to about 36, about 24 to about 38, about 24 to about 40, about 26 to about 28, about 26 to about 30, about 26 to about 32, about 26 to about 34, about 26 to about 36, about 26 to about 38, about 26 to about 40, about 28 to about 30, about 28 to about 32, about 28 to about 34, about 28 to about 36, about 28 to about 38, about 28 to about 40, about 30 to about 32, about 30 to about 34, about 30 to about 36, about 30 to about 38, about 30 to about 40, about 32 to about 34, about 32 to about 36, about 32 to about 38, about 32 to about 40, about 34 to about 36, about 34 to about 38, about 34 to about 40, about 36 to about 38, about 36 to about 40, or about 38 to about 40. In some embodiments, the CNR may be at least 20.2, e.g., about 20.2, about 21.1, about 22, about 24, about 26, about 28, about 30, about 32, about 34, about 36, about 38, or about 40. In some embodiments, the CNR may be at least 20.2, e.g., at least about 20.2, about 21.1, about 22, about 24, about 26, about 28, about 30, about 32, about 34, about 36, or about 38. In some embodiments, the CNR may be at least 20.2, e.g., at most about 21.1, about 22, about 24, about 26, about 28, about 30, about 32, about 34, about 36, about 38, or about 40.

The viscoelastic media with the precious metal may have improved effectiveness in various injection sites due to increased or decreased noise values at the various injection sites. For example, the noise value may be relatively high in the abdomen, but relatively low in the breast or rectum. Thus, in particular, the viscoelastic media is effective as a fiducial marker in breast and rectum injection sites. While the breast and the rectum are described as injection sites where the viscoelastic media may be used effectively as a fiducial marker, the breast and the rectum are exemplary, and the viscoelastic media may be used effectively as a fiducial marker at other injection sites as well.

Further, viscoelastic media with the visual additive may migrate through or be absorbed into specific tissues. In some embodiments, the migration or absorption may be measured. In some embodiments, the migration or absorption may be measured by dispersion of the viscoelastic media with the visual additive. The dispersion may be measured as migration of the viscoelastic media, where the migration is the change in position in the tissue of the viscoelastic media particles. In some cases, the migration may be measured as the degradation of image contrast. The degradation of image contrast may be measured as a difference between the contrast of an image at a starting point and the contrast of an image at a later point. For example, the degradation of image contrast may be measured as a distance between the difference in grayscale of the injected tissue and grayscale of the tissue surrounding the injected tissue at first time (e.g., the contrast at a starting point) and the difference in grayscale of the injected tissue and the grayscale of the tissue surrounding the injected tissue at a second time (e.g., the contrast at a later point).

In some embodiments, dispersion in the form of migration may not occur (e.g., the viscoelastic media particles do not move within the tissue) over time. In these embodiments, the viscoelastic media particles may be evenly distributed with no clumping. Further, the viscoelastic media may have an amount of about 0.5 ml to about 30 ml.

In some embodiments, dispersion in the form of migration may be between about 0.001 mm and about 0.01 mm. In some embodiments, the dispersion in the form of migration may be about 0.001 mm to about 0.01 mm. In some embodiments, the dispersion in the form of migration may be about 0.001 mm to about 0.002 mm, about 0.001 mm to about 0.003 mm, about 0.001 mm to about 0.004 mm, about 0.001 mm to about 0.005 mm, about 0.001 mm to about 0.006 mm, about 0.001 mm to about 0.007 mm, about 0.001 mm to about 0.008 mm, about 0.001 mm to about 0.009 mm, about 0.001 mm to about 0.01 mm, about 0.002 mm to about 0.003 mm, about 0.002 mm to about 0.004 mm, about 0.002 mm to about 0.005 mm, about 0.002 mm to about 0.006 mm, about 0.002 mm to about 0.007 mm, about 0.002 mm to about 0.008 mm, about 0.002 mm to about 0.009 mm, about 0.002 mm to about 0.01 mm, about 0.003 mm to about 0.004 mm, about 0.003 mm to about 0.005 mm, about 0.003 mm to about 0.006 mm, about 0.003 mm to about 0.007 mm, about 0.003 mm to about 0.008 mm, about 0.003 mm to about 0.009 mm, about 0.003 mm to about 0.01 mm, about 0.004 mm to about 0.005 mm, about 0.004 mm to about 0.006 mm, about 0.004 mm to about 0.007 mm, about 0.004 mm to about 0.008 mm, about 0.004 mm to about 0.009 mm, about 0.004 mm to about 0.01 mm, about 0.005 mm to about 0.006 mm, about 0.005 mm to about 0.007 mm, about 0.005 mm to about 0.008 mm, about 0.005 mm to about 0.009 mm, about 0.005 mm to about 0.01 mm, about 0.006 mm to about 0.007 mm, about 0.006 mm to about 0.008 mm, about 0.006 mm to about 0.009 mm, about 0.006 mm to about 0.01 mm, about 0.007 mm to about 0.008 mm, about 0.007 mm to about 0.009 mm, about 0.007 mm to about 0.01 mm, about 0.008 mm to about 0.009 mm, about 0.008 mm to about 0.01 mm, or about 0.009 mm to about 0.01 mm. In some embodiments, the dispersion in the form of migration may be about 0.001 mm, about 0.002 mm, about 0.003 mm, about 0.004 mm, about 0.005 mm, about 0.006 mm, about 0.007 mm, about 0.008 mm, about 0.009 mm, or about 0.01 mm. In some embodiments, the dispersion in the form of migration may be at least about 0.001 mm, about 0.002 mm, about 0.003 mm, about 0.004 mm, about 0.005 mm, about 0.006 mm, about 0.007 mm, about 0.008 mm, or about 0.009 mm. In some embodiments, the dispersion in the form of migration may be at most about 0.002 mm, about 0.003 mm, about 0.004 mm, about 0.005 mm, about 0.006 mm, about 0.007 mm, about 0.008 mm, about 0.009 mm, or about 0.01 mm.

In some embodiments, the dispersion in the form of migration may be about 0.01 mm to about 0.1 mm. In some embodiments, the dispersion in the form of migration may be about 0.01 mm to about 0.02 mm, about 0.01 mm to about 0.03 mm, about 0.01 mm to about 0.04 mm, about 0.01 mm to about 0.05 mm, about 0.01 mm to about 0.06 mm, about 0.01 mm to about 0.07 mm, about 0.01 mm to about 0.08 mm, about 0.01 mm to about 0.09 mm, about 0.01 mm to about 0.1 mm, about 0.02 mm to about 0.03 mm, about 0.02 mm to about 0.04 mm, about 0.02 mm to about 0.05 mm, about 0.02 mm to about 0.06 mm, about 0.02 mm to about 0.07 mm, about 0.02 mm to about 0.08 mm, about 0.02 mm to about 0.09 mm, about 0.02 mm to about 0.1 mm, about 0.03 mm to about 0.04 mm, about 0.03 mm to about 0.05 mm, about 0.03 mm to about 0.06 mm, about 0.03 mm to about 0.07 mm, about 0.03 mm to about 0.08 mm, about 0.03 mm to about 0.09 mm, about 0.03 mm to about 0.1 mm, about 0.04 mm to about 0.05 mm, about 0.04 mm to about 0.06 mm, about 0.04 mm to about 0.07 mm, about 0.04 mm to about 0.08 mm, about 0.04 mm to about 0.09 mm, about 0.04 mm to about 0.1 mm, about 0.05 mm to about 0.06 mm, about 0.05 mm to about 0.07 mm, about 0.05 mm to about 0.08 mm, about 0.05 mm to about 0.09 mm, about 0.05 mm to about 0.1 mm, about 0.06 mm to about 0.07 mm, about 0.06 mm to about 0.08 mm, about 0.06 mm to about 0.09 mm, about 0.06 mm to about 0.1 mm, about 0.07 mm to about 0.08 mm, about 0.07 mm to about 0.09 mm, about 0.07 mm to about 0.1 mm, about 0.08 mm to about 0.09 mm, about 0.08 mm to about 0.1 mm, or about 0.09 mm to about 0.1 mm. In some embodiments, the dispersion in the form of migration may be about 0.01 mm, about 0.02 mm, about 0.03 mm, about 0.04 mm, about 0.05 mm, about 0.06 mm, about 0.07 mm, about 0.08 mm, about 0.09 mm, or about 0.1 mm. In some embodiments, the dispersion in the form of migration may be at least about 0.01 mm, about 0.02 mm, about 0.03 mm, about 0.04 mm, about 0.05 mm, about 0.06 mm, about 0.07 mm, about 0.08 mm, or about 0.09 mm. In some embodiments, the dispersion in the form of migration may be at most about 0.02 mm, about 0.03 mm, about 0.04 mm, about 0.05 mm, about 0.06 mm, about 0.07 mm, about 0.08 mm, about 0.09 mm, or about 0.1 mm.

In some embodiments, dispersion in the form of migration may be between about 0.1 mm and about 1.0 mm. In some embodiments, the dispersion in the form of migration may be about 0.1 mm to about 1 mm. In some embodiments, the dispersion in the form of migration may be about 0.1 mm to about 0.2 mm, about 0.1 mm to about 0.3 mm, about 0.1 mm to about 0.4 mm, about 0.1 mm to about 0.5 mm, about 0.1 mm to about 0.6 mm, about 0.1 mm to about 0.7 mm, about 0.1 mm to about 0.8 mm, about 0.1 mm to about 0.9 mm, about 0.1 mm to about 1 mm, about 0.2 mm to about 0.3 mm, about 0.2 mm to about 0.4 mm, about 0.2 mm to about 0.5 mm, about 0.2 mm to about 0.6 mm, about 0.2 mm to about 0.7 mm, about 0.2 mm to about 0.8 mm, about 0.2 mm to about 0.9 mm, about 0.2 mm to about 1 mm, about 0.3 mm to about 0.4 mm, about 0.3 mm to about 0.5 mm, about 0.3 mm to about 0.6 mm, about 0.3 mm to about 0.7 mm, about 0.3 mm to about 0.8 mm, about 0.3 mm to about 0.9 mm, about 0.3 mm to about 1 mm, about 0.4 mm to about 0.5 mm, about 0.4 mm to about 0.6 mm, about 0.4 mm to about 0.7 mm, about 0.4 mm to about 0.8 mm, about 0.4 mm to about 0.9 mm, about 0.4 mm to about 1 mm, about 0.5 mm to about 0.6 mm, about 0.5 mm to about 0.7 mm, about 0.5 mm to about 0.8 mm, about 0.5 mm to about 0.9 mm, about 0.5 mm to about 1 mm, about 0.6 mm to about 0.7 mm, about 0.6 mm to about 0.8 mm, about 0.6 mm to about 0.9 mm, about 0.6 mm to about 1 mm, about 0.7 mm to about 0.8 mm, about 0.7 mm to about 0.9 mm, about 0.7 mm to about 1 mm, about 0.8 mm to about 0.9 mm, about 0.8 mm to about 1 mm, or about 0.9 mm to about 1 mm. In some embodiments, the dispersion in the form of migration may be about 0.1 mm, about 0.2 mm, about 0.3 mm, about 0.4 mm, about 0.5 mm, about 0.6 mm, about 0.7 mm, about 0.8 mm, about 0.9 mm, or about 1 mm. In some embodiments, the dispersion in the form of migration may be at least about 0.1 mm, about 0.2 mm, about 0.3 mm, about 0.4 mm, about 0.5 mm, about 0.6 mm, about 0.7 mm, about 0.8 mm, or about 0.9 mm. In some embodiments, the dispersion in the form of migration may be at most about 0.2 mm, about 0.3 mm, about 0.4 mm, about 0.5 mm, about 0.6 mm, about 0.7 mm, about 0.8 mm, about 0.9 mm, or about 1 mm.

In some embodiments, dispersion in the form of migration may be between about 1.0 mm and about 2.0 mm. In some embodiments, the dispersion in the form of migration may be about 1 mm to about 2 mm. In some embodiments, the dispersion in the form of migration may be about 1 mm to about 1.1 mm, about 1 mm to about 1.2 mm, about 1 mm to about 1.3 mm, about 1 mm to about 1.4 mm, about 1 mm to about 1.5 mm, about 1 mm to about 1.6 mm, about 1 mm to about 1.7 mm, about 1 mm to about 1.8 mm, about 1 mm to about 1.9 mm, about 1 mm to about 2 mm, about 1.1 mm to about 1.2 mm, about 1.1 mm to about 1.3 mm, about 1.1 mm to about 1.4 mm, about 1.1 mm to about 1.5 mm, about 1.1 mm to about 1.6 mm, about 1.1 mm to about 1.7 mm, about 1.1 mm to about 1.8 mm, about 1.1 mm to about 1.9 mm, about 1.1 mm to about 2 mm, about 1.2 mm to about 1.3 mm, about 1.2 mm to about 1.4 mm, about 1.2 mm to about 1.5 mm, about 1.2 mm to about 1.6 mm, about 1.2 mm to about 1.7 mm, about 1.2 mm to about 1.8 mm, about 1.2 mm to about 1.9 mm, about 1.2 mm to about 2 mm, about 1.3 mm to about 1.4 mm, about 1.3 mm to about 1.5 mm, about 1.3 mm to about 1.6 mm, about 1.3 mm to about 1.7 mm, about 1.3 mm to about 1.8 mm, about 1.3 mm to about 1.9 mm, about 1.3 mm to about 2 mm, about 1.4 mm to about 1.5 mm, about 1.4 mm to about 1.6 mm, about 1.4 mm to about 1.7 mm, about 1.4 mm to about 1.8 mm, about 1.4 mm to about 1.9 mm, about 1.4 mm to about 2 mm, about 1.5 mm to about 1.6 mm, about 1.5 mm to about 1.7 mm, about 1.5 mm to about 1.8 mm, about 1.5 mm to about 1.9 mm, about 1.5 mm to about 2 mm, about 1.6 mm to about 1.7 mm, about 1.6 mm to about 1.8 mm, about 1.6 mm to about 1.9 mm, about 1.6 mm to about 2 mm, about 1.7 mm to about 1.8 mm, about 1.7 mm to about 1.9 mm, about 1.7 mm to about 2 mm, about 1.8 mm to about 1.9 mm, about 1.8 mm to about 2 mm, or about 1.9 mm to about 2 mm. In some embodiments, the dispersion in the form of migration may be about 1 mm, about 1.1 mm, about 1.2 mm, about 1.3 mm, about 1.4 mm, about 1.5 mm, about 1.6 mm, about 1.7 mm, about 1.8 mm, about 1.9 mm, or about 2 mm. In some embodiments, the dispersion in the form of migration may be at least about 1 mm, about 1.1 mm, about 1.2 mm, about 1.3 mm, about 1.4 mm, about 1.5 mm, about 1.6 mm, about 1.7 mm, about 1.8 mm, or about 1.9 mm. In some embodiments, the dispersion in the form of migration may be at most about 1.1 mm, about 1.2 mm, about 1.3 mm, about 1.4 mm, about 1.5 mm, about 1.6 mm, about 1.7 mm, about 1.8 mm, about 1.9 mm, or about 2 mm.

The dispersion may also be measured as the deformation of the viscoelastic media with the visual additive in the injected tissue. The deformation may be measured as a difference along radial lines between an image at a starting point and an image at a later point. For example, the radial lines of a first image of the injected tissue and the surrounding tissue taken at a starting point and a second image of the injected tissue and the surrounding tissue taken at a later point may be determined, and the deformation may be determined as the movement or difference of the corresponding radial lines of the two images. In those embodiments, the deformation may be measured as a percentage (%) difference or a difference in distance (e.g., mm or cm).

The dispersion may also be measured as the resorption of the viscoelastic media in the injected tissue and the surrounding tissue. The resorption may be measured as the percentage loss in volume of the marker. For example, a first image of the injected tissue and the surrounding tissue may be taken at a starting point and a second image of the injected tissue and the surrounding tissue may be taken at a later point. The volume of the marker may be determined at the starting point and the later point based on the contrast of the two images as described above, and the percentage loss in volume may be determined based on the difference between the volume at the starting point and the volume at the later point.

In some embodiments, the viscoelastic media particles with the visual additives may be resorbed into the tissue without losing the original shape of the particles after injection.

Additionally, in some embodiments, the viscoelastic media particles with the visual additives may not be resorbed into tissue over time.

In some embodiments, the viscoelastic media particles may be resorbed into tissue over a time period of about 3 months to about 36 months. In some embodiments, the viscoelastic media particles may be resorbed into tissue over a time period of about 3 months to about 6 months, about 3 months to about 9 months, about 3 months to about 12 months, about 3 months to about 15 months, about 3 months to about 18 months, about 3 months to about 21 months, about 3 months to about 24 months, about 3 months to about 27 months, about 3 months to about 30 months, about 3 months to about 33 months, about 3 months to about 36 months, about 6 months to about 9 months, about 6 months to about 12 months, about 6 months to about 15 months, about 6 months to about 18 months, about 6 months to about 21 months, about 6 months to about 24 months, about 6 months to about 27 months, about 6 months to about 30 months, about 6 months to about 33 months, about 6 months to about 36 months, about 9 months to about 12 months, about 9 months to about 15 months, about 9 months to about 18 months, about 9 months to about 21 months, about 9 months to about 24 months, about 9 months to about 27 months, about 9 months to about 30 months, about 9 months to about 33 months, about 9 months to about 36 months, about 12 months to about 15 months, about 12 months to about 18 months, about 12 months to about 21 months, about 12 months to about 24 months, about 12 months to about 27 months, about 12 months to about 30 months, about 12 months to about 33 months, about 12 months to about 36 months, about 15 months to about 18 months, about 15 months to about 21 months, about 15 months to about 24 months, about 15 months to about 27 months, about 15 months to about 30 months, about 15 months to about 33 months, about 15 months to about 36 months, about 18 months to about 21 months, about 18 months to about 24 months, about 18 months to about 27 months, about 18 months to about 30 months, about 18 months to about 33 months, about 18 months to about 36 months, about 21 months to about 24 months, about 21 months to about 27 months, about 21 months to about 30 months, about 21 months to about 33 months, about 21 months to about 36 months, about 24 months to about 27 months, about 24 months to about 30 months, about 24 months to about 33 months, about 24 months to about 36 months, about 27 months to about 30 months, about 27 months to about 33 months, about 27 months to about 36 months, about 30 months to about 33 months, about 30 months to about 36 months, or about 33 months to about 36 months. In some embodiments, the viscoelastic media particles may be resorbed into tissue over a time period of about 3 months, about 6 months, about 9 months, about 12 months, about 15 months, about 18 months, about 21 months, about 24 months, about 27 months, about 30 months, about 33 months, or about 36 months. In some embodiments, the viscoelastic media particles may be resorbed into tissue over a time period of at least about 3 months, about 6 months, about 9 months, about 12 months, about 15 months, about 18 months, about 21 months, about 24 months, about 27 months, about 30 months, or about 33 months. In some embodiments, the viscoelastic media particles may be resorbed into tissue over a time period of at most about 6 months, about 9 months, about 12 months, about 15 months, about 18 months, about 21 months, about 24 months, about 27 months, about 30 months, about 33 months, or about 36 months.

In some embodiments, the viscoelastic media particles may be resorbed into tissue over a time period of about 12 months to about 16 months. In some embodiments, the viscoelastic media particles may be resorbed into tissue over a time period of about 12 months to about 12.5 months, about 12 months to about 13 months, about 12 months to about 13.5 months, about 12 months to about 14 months, about 12 months to about 14.5 months, about 12 months to about 15 months, about 12 months to about 15.5 months, about 12 months to about 16 months, about 12.5 months to about 13 months, about 12.5 months to about 13.5 months, about 12.5 months to about 14 months, about 12.5 months to about 14.5 months, about 12.5 months to about 15 months, about 12.5 months to about 15.5 months, about 12.5 months to about 16 months, about 13 months to about 13.5 months, about 13 months to about 14 months, about 13 months to about 14.5 months, about 13 months to about 15 months, about 13 months to about 15.5 months, about 13 months to about 16 months, about 13.5 months to about 14 months, about 13.5 months to about 14.5 months, about 13.5 months to about 15 months, about 13.5 months to about 15.5 months, about 13.5 months to about 16 months, about 14 months to about 14.5 months, about 14 months to about 15 months, about 14 months to about 15.5 months, about 14 months to about 16 months, about 14.5 months to about 15 months, about 14.5 months to about 15.5 months, about 14.5 months to about 16 months, about 15 months to about 15.5 months, about 15 months to about 16 months, or about 15.5 months to about 16 months. In some embodiments, the viscoelastic media particles may be resorbed into tissue over a time period of about 12 months, about 12.5 months, about 13 months, about 13.5 months, about 14 months, about 14.5 months, about 15 months, about 15.5 months, or about 16 months. In some embodiments, the viscoelastic media particles may be resorbed into tissue over a time period of at least about 12 months, about 12.5 months, about 13 months, about 13.5 months, about 14 months, about 14.5 months, about 15 months, or about 15.5 months. In some embodiments, the viscoelastic media particles may be resorbed into tissue over a time period of at most about 12.5 months, about 13 months, about 13.5 months, about 14 months, about 14.5 months, about 15 months, about 15.5 months, or about 16 months.

In some aspects, the length of the period of time of which the viscoelastic media particles with the visual additive particles gets resorbed improves the effectiveness of the viscoelastic media particles because visibility is increased for a longer period of time and re-administration of viscoelastic media particles with the visual additive particles is less frequent than as needed by other products, if at all. In some embodiments, the viscoelastic media particles with the visual additive particles is injected once every about 3 months to about 36 months. In some embodiments, the viscoelastic media particles with the visual additive particles is injected once every about 3 months to about 6 months, about 3 months to about 9 months, about 3 months to about 12 months, about 3 months to about 15 months, about 3 months to about 18 months, about 3 months to about 21 months, about 3 months to about 24 months, about 3 months to about 27 months, about 3 months to about 30 months, about 3 months to about 33 months, about 3 months to about 36 months, about 6 months to about 9 months, about 6 months to about 12 months, about 6 months to about 15 months, about 6 months to about 18 months, about 6 months to about 21 months, about 6 months to about 24 months, about 6 months to about 27 months, about 6 months to about 30 months, about 6 months to about 33 months, about 6 months to about 36 months, about 9 months to about 12 months, about 9 months to about 15 months, about 9 months to about 18 months, about 9 months to about 21 months, about 9 months to about 24 months, about 9 months to about 27 months, about 9 months to about 30 months, about 9 months to about 33 months, about 9 months to about 36 months, about 12 months to about 15 months, about 12 months to about 18 months, about 12 months to about 21 months, about 12 months to about 24 months, about 12 months to about 27 months, about 12 months to about 30 months, about 12 months to about 33 months, about 12 months to about 36 months, about 15 months to about 18 months, about 15 months to about 21 months, about 15 months to about 24 months, about 15 months to about 27 months, about 15 months to about 30 months, about 15 months to about 33 months, about 15 months to about 36 months, about 18 months to about 21 months, about 18 months to about 24 months, about 18 months to about 27 months, about 18 months to about 30 months, about 18 months to about 33 months, about 18 months to about 36 months, about 21 months to about 24 months, about 21 months to about 27 months, about 21 months to about 30 months, about 21 months to about 33 months, about 21 months to about 36 months, about 24 months to about 27 months, about 24 months to about 30 months, about 24 months to about 33 months, about 24 months to about 36 months, about 27 months to about 30 months, about 27 months to about 33 months, about 27 months to about 36 months, about 30 months to about 33 months, about 30 months to about 36 months, or about 33 months to about 36 months. In some embodiments, the viscoelastic media particles with the visual additive particles is injected once every about 3 months, about 6 months, about 9 months, about 12 months, about 15 months, about 18 months, about 21 months, about 24 months, about 27 months, about 30 months, about 33 months, or about 36 months. In some embodiments, the viscoelastic media particles with the visual additive particles is injected once every at least about 3 months, about 6 months, about 9 months, about 12 months, about 15 months, about 18 months, about 21 months, about 24 months, about 27 months, about 30 months, or about 33 months. In some embodiments, the viscoelastic media particles with the visual additive particles is injected once every at most about 6 months, about 9 months, about 12 months, about 15 months, about 18 months, about 21 months, about 24 months, about 27 months, about 30 months, about 33 months, or about 36 months.

In some embodiments, the viscoelastic media with the visual additive may appear (e.g., be visible) during the injection or immediately after the injection of the viscoelastic media to tissue. In other embodiments, the viscoelastic media with the visual additive may appear after a period of time following the injection (e.g., up to thirty minutes after the injection).

Additionally, the addition of the visual additive to the viscoelastic media improves visibility of the viscoelastic media, and therefore, allows the movement of the viscoelastic media to be more easily determined. For example, the movement of the viscoelastic media may be more easily determined due to the contrast of the images, which are clearer and more easily obtained due to the improved visibility of the viscoelastic media. Even further, the addition of the visual additive may affect the movement based on characteristics of the visual additive (e.g., size or composition). For example, if the size of a particle of the visual additive is about 80 micrometers to about 120 micrometers in diameter, the viscoelastic media with the visual additive may not move through the tissue as much as viscoelastic media with visual additives having smaller particle sizes. Further, viscoelastic media with visual additive particles in the size range of about 80 micrometers to about 120 micrometers in diameter show less accumulation in organs throughout the body (e.g., liver, spleen, lungs, brain, stomach, and/or pancreas) due to decreased migration. Addition size ranges of the visual additive particles (e.g., about 15 micrometers to about 200 micrometers, about 15 micrometers to about 50 micrometers, and/or about 120 micrometers to about 200 micrometers) with may be associated with different amounts of accumulation in organs and migration. While certain size ranges and characteristics for visual additives are described above, these size ranges and characteristics are exemplary, and other size ranges and characteristics may be used.

In some embodiments, the amount of visual additive in the viscoelastic media may increase the visibility of the viscoelastic media. In some embodiments, about 15 mg to about 30 mg of visual additive may be added to the viscoelastic media per ml of viscoelastic media. In some embodiments, the weight percentage of the visual additive within the viscoelastic media may be about 0.01% to about 0.25%. In some embodiments, the weight percentage of the visual additive within the viscoelastic media may be about 0.01% to about 0.04%, about 0.01% to about 0.07%, about 0.01% to about 0.1%, about 0.01% to about 0.13%, about 0.01% to about 0.16%, about 0.01% to about 0.19%, about 0.01% to about 0.22%, about 0.01% to about 0.25%, about 0.04% to about 0.07%, about 0.04% to about 0.10%, about 0.04% to about 0.13%, about 0.04% to about 0.16%, about 0.04% to about 0.19%, about 0.04% to about 0.22%, about 0.04% to about 0.25%, about 0.07% to about 0.10%, about 0.07% to about 0.13%, about 0.07% to about 0.16%, about 0.07% to about 0.19%, about 0.07% to about 0.22%, about 0.07% to about 0.25%, about 0.1% to about 0.13%, about 0.1% to about 0.16%, about 0.1% to about 0.19%, about 0.1% to about 0.22%, about 0.1% to about 0.25%, about 0.13% to about 0.16%, about 0.13% to about 0.19%, about 0.13% to about 0.22%, about 0.13% to about 0.25%, about 0.16% to about 0.19%, about 0.16% to about 0.22%, about 0.16% to about 0.25%, about 0.19% to about 0.22%, about 0.19% to about 0.25%, or about 0.22% to about 0.25%. In some embodiments, the weight percentage of the visual additive within the viscoelastic media may be about 0.01%, about 0.04%, about 0.07%, about 0.1%, about 0.13%, about 0.16%, about 0.19%, about 0.22%, or about 0.25%. In some embodiments, the weight percentage of the visual additive within the viscoelastic media may be at least about 0.01%, about 0.04%, about 0.07%, about 0.1%, about 0.13%, about 0.16%, about 0.19%, or about 0.22%. In some embodiments, the weight percentage of the visual additive within the viscoelastic media may be at most about 0.04%, about 0.07%, about 0.10%, about 0.13%, about 0.16%, about 0.19%, about 0.22%, or about 0.25%. In some embodiments, the weight percentage of the visual additive within the viscoelastic media may be about 0.07% (w/w) to about 0.15% (w/w). In some embodiments, the weight percentage of the visual additive within the viscoelastic media may be about 0.07% to about 0.08%, about 0.07% to about 0.09%, about 0.07% to about 0.1%, about 0.07% to about 0.110%, about 0.07% to about 0.12%, about 0.07% to about 0.13%, about 0.07% to about 0.14%, about 0.07% to about 0.15%, about 0.08% to about 0.09%, about 0.08% to about 0.1%, about 0.08% to about 0.11%, about 0.08% to about 0.12%, about 0.08% to about 0.13%, about 0.08% to about 0.14%, about 0.08% to about 0.15%, about 0.09% to about 0.1%, about 0.09% to about 0.11%, about 0.09% to about 0.12%, about 0.09% to about 0.13%, about 0.09% to about 0.14%, about 0.09% to about 0.15%, about 0.1% to about 0.11%, about 0.1% to about 0.12%, about 0.1% to about 0.13%, about 0.1% to about 0.14%, about 0.1% to about 0.15%, about 0.11% to about 0.12%, about 0.11% to about 0.13%, about 0.11% to about 0.14%, about 0.110% to about 0.15%, about 0.12% to about 0.13%, about 0.12% to about 0.14%, about 0.12% to about 0.15%, about 0.13% to about 0.14%, about 0.13% to about 0.15%, or about 0.14% to about 0.15%. In some embodiments, the weight percentage of the visual additive within the viscoelastic media may be about 0.07%, about 0.08%, about 0.09%, about 0.1%, about 0.11%, about 0.12%, about 0.13%, about 0.14%, or about 0.15%. In some embodiments, the weight percentage of the visual additive within the viscoelastic media may be at least about 0.07%, about 0.08%, about 0.09%, about 0.1%, about 0.11%, about 0.12%, about 0.13%, or about 0.14%. In some embodiments, the weight percentage of the visual additive within the viscoelastic media may be at most about 0.08%, about 0.09%, about 0.1%, about 0.11%, about 0.12%, about 0.13%, about 0.14%, or about 0.15%.

### Image Enhancement

As polyethylene glycol, hyaluronic acid, and NASHA gels can image poorly on CT scans, MRI, and TRUS (transrectal ultrasound), the additives (e.g., visual additives) and compositions are configured to allow a clinician to inject the organ spacing materials herein at an accurate location, through real-time scanning feedback (e.g., CNR). Further, such scans can be used for radiation planning. HA CT imaging enhancement would be a very significant feature, sparing the need for a patient MRI, cost, and the CT/MRI fusion step to treatment plan. Additionally, for boost and Accelerated Partial Breast Irradiation (APBI) planning it is very important to have a clear identification of seroma to enable accurate target volume contouring and to initiate cone beam image guided radiotherapy. However, as seroma is not always visible on CT-simulation, many surgeries, such as those requiring full thickness closure at time of surgery, are difficult to plan and can be ineligible for such APBI procedures. While clip placement at time of surgery has been used to aid such contouring, they often form unreliable, whereas high-Z clip materials deform the contouring images and low-Z clip materials, such as tantalum, are not visible.

However, the image enhancement agents provided herein exhibit a Z value that is sufficiently to enable perfect visibility on CT and paramagnetic moment for visibility on MRI, but not too high to avoid the degradation of seroma imaging. Further optimal visibility and image quality is achieved by varying the volume of the injected image enhancement agents while maintaining minimal expansion of the volume to be treated.

In one embodiment, the visualization additive comprises Iodine which enhances CT imaging, but may have no benefit to MRI and TRUS imaging. Further, Iodine can be an allergen.

In some embodiments, the visualization additive (also referred to as “the visual additive” above) comprises a radiopaque compound selected from the group consisting of gold, iodine, gadolinium, iron, barium, calcium, magnesium, or any combination thereof. In some embodiments, the gold visualization additive comprises gold particles. In some embodiments, a concentration of the visualization additive in the polyethylene glycol, the hyaluronic acid, or both is about 15 mg/ml to about 30 mg/ml. In some embodiments, a concentration of the visualization additive in the spacer materials is about 0.010% to about 1.5% (w/w). In some embodiments, a concentration of the visualization additive in the spacer materials is at least about 0.01% to about 1.5% (w/w), e.g., about 0.01% to about 1.0%, about 0.01% to about 0.75%, about 0.01% to about 0.5%, about 0.01% to about 0.25%, about 0.01% to about 0.15%, about 0.01% to about 0.05%, about 0.01% to about 0.04%, about 0.01% to about 0.03%, about 0.01% to about 0.02%, about 0.02% to about 1.5%, about 0.02% to about 1.0%, about 0.02% to about 0.75%, about 0.02% to about 0.5%, about 0.02% to about 0.25%, about 0.02% to about 0.15%, about 0.02% to about 0.05%, about 0.02% to about 0.04%, about 0.02% to about 0.03%, about 0.03% to about 1.5%, about 0.03% to about 1.0%, about 0.03% to about 0.75%, about 0.03% to about 0.5%, about 0.03% to about 0.25%, about 0.03% to about 0.15%, about 0.03% to about 0.05%, about 0.03% to about 0.04%, about 0.04% to about 1.5%, about 0.04% to about 1.0%, about 0.04% to about 0.75%, about 0.04% to about 0.5%, about 0.04% to about 0.25%, about 0.04% to about 0.15%, about 0.04% to about 0.05%, about 0.05% to about 1.5%, about 0.05% to about 1.0%, about 0.05% to about 0.75%, about 0.05% to about 0.5%, about 0.05% to about 0.25%, about 0.05% to about 0.15%, about 0.15% to about 1.5%, about 0.15% to about 1.0%, about 0.15% to about 0.75%, about 0.15% to about 0.5%, about 0.15% to about 0.25%, about 0.25% to about 1.5%, about 0.25% to about 1.0%, about 0.25% to about 0.75%, about 0.25% to about 0.5%, about 0.50% to about 1.5%, about 0.50% to about 1.0%, about 0.50% to about 0.75%, about 0.75% to about 1.5%, about 0.75% to about 1.0%, about 1.0% to about 1.5%, about 0.01%, about 0.02%, about 0.03%, about 0.04%, about 0.05%, about 0.06%, about 0.07%, about 0.08%, about 0.09%, about 0.1%, about 0.15%, about 0.25%, about 0.50%, about 0.75%, about 1.0%, or about 1.5%. In some embodiments, a concentration of the visualization additive in the spacer material is about 0.1% to about 15%. In some embodiments, a concentration of the visualization additive in the spacer material is at least about 0.1% the visual additive is present in the gel at about 0.5 mg/ml of gel to about 1 mg/ml of gel, about 0.5 mg/ml of gel to about 1.5 mg/ml of gel, about 0.5 mg/ml of gel to about 2 mg/ml of gel, about 0.5 mg/ml of gel to about 2.5 mg/ml of gel, about 0.5 mg/ml of gel to about 3 mg/ml of gel, about 0.5 mg/ml of gel to about 3.5 mg/ml of gel, about 0.5 mg/ml of gel to about 4 mg/ml of gel, about 0.5 mg/ml of gel to about 4.5 mg/ml of gel, about 0.5 mg/ml of gel to about 5 mg/ml of gel, about 0.5 mg/ml of gel to about 5.5 mg/ml of gel, about 0.5 mg/ml of gel to about 6 mg/ml of gel, about 1 mg/ml of gel to about 1.5 mg/ml of gel, about 1 mg/ml of gel to about 2 mg/ml of gel, about 1 mg/ml of gel to about 2.5 mg/ml of gel, about 1 mg/ml of gel to about 3 mg/ml of gel, about 1 mg/ml of gel to about 3.5 mg/ml of gel, about 1 mg/ml of gel to about 4 mg/ml of gel, about 1 mg/ml of gel to about 4.5 mg/ml of gel, about 1 mg/ml of gel to about 5 mg/ml of gel, about 1 mg/ml of gel to about 5.5 mg/ml of gel, about 1 mg/ml of gel to about 6 mg/ml of gel, about 1.5 mg/ml of gel to about 2 mg/ml of gel, about 1.5 mg/ml of gel to about 2.5 mg/ml of gel, about 1.5 mg/ml of gel to about 3 mg/ml of gel, about 1.5 mg/ml of gel to about 3.5 mg/ml of gel, about 1.5 mg/ml of gel to about 4 mg/ml of gel, about 1.5 mg/ml of gel to about 4.5 mg/ml of gel, about 1.5 mg/ml of gel to about 5 mg/ml of gel, about 1.5 mg/ml of gel to about 5.5 mg/ml of gel, about 1.5 mg/ml of gel to about 6 mg/ml of gel, about 2 mg/ml of gel to about 2.5 mg/ml of gel, about 2 mg/ml of gel to about 3 mg/ml of gel, about 2 mg/ml of gel to about 3.5 mg/ml of gel, about 2 mg/ml of gel to about 4 mg/ml of gel, about 2 mg/ml of gel to about 4.5 mg/ml of gel, about 2 mg/ml of gel to about 5 mg/ml of gel, about 2 mg/ml of gel to about 5.5 mg/ml of gel, about 2 mg/ml of gel to about 6 mg/ml of gel, about 2.5 mg/ml of gel to about 3 mg/ml of gel, about 2.5 mg/ml of gel to about 3.5 mg/ml of gel, about 2.5 mg/ml of gel to about 4 mg/ml of gel, about 2.5 mg/ml of gel to about 4.5 mg/ml of gel, about 2.5 mg/ml of gel to about 5 mg/ml of gel, about 2.5 mg/ml of gel to about 5.5 mg/ml of gel, about 2.5 mg/ml of gel to about 6 mg/ml of gel, about 3 mg/ml of gel to about 3.5 mg/ml of gel, about 3 mg/ml of gel to about 4 mg/ml of gel, about 3 mg/ml of gel to about 4.5 mg/ml of gel, about 3 mg/ml of gel to about 5 mg/ml of gel, about 3 mg/ml of gel to about 5.5 mg/ml of gel, about 3 mg/ml of gel to about 6 mg/ml of gel, about 3.5 mg/ml of gel to about 4 mg/ml of gel, about 3.5 mg/ml of gel to about 4.5 mg/ml of gel, about 3.5 mg/ml of gel to about 5 mg/ml of gel, about 3.5 mg/ml of gel to about 5.5 mg/ml of gel, about 3.5 mg/ml of gel to about 6 mg/ml of gel, about 4 mg/ml of gel to about 4.5 mg/ml of gel, about 4 mg/ml of gel to about 5 mg/ml of gel, about 4 mg/ml of gel to about 5.5 mg/ml of gel, about 4 mg/ml of gel to about 6 mg/ml of gel, about 4.5 mg/ml of gel to about 5 mg/ml of gel, about 4.5 mg/ml of gel to about 5.5 mg/ml of gel, about 4.5 mg/ml of gel to about 6 mg/ml of gel, about 5 mg/ml of gel to about 5.5 mg/ml of gel, about 5 mg/ml of gel to about 6 mg/ml of gel, or about 5.5 mg/ml of gel to about 6 mg/ml of gel. In some embodiments, the visual additive is present in the gel at about 0.5 mg/ml of gel, about 1 mg/ml of gel, about 1.5 mg/ml of gel, about 2 mg/ml of gel, about 2.5 mg/ml of gel, about 3 mg/ml of gel, about 3.5 mg/ml of gel, about 4 mg/ml of gel, about 4.5 mg/ml of gel, about 5 mg/ml of gel, about 5.5 mg/ml of gel, or about 6 mg/ml of gel. In some embodiments, the visualization additive is present in the gel at least about 0.5 mg/ml of gel, about 1 mg/ml of gel, about 1.5 mg/ml of gel, about 2 mg/ml of gel, about 2.5 mg/ml of gel, about 3 mg/ml of gel, about 3.5 mg/ml of gel, about 4 mg/ml of gel, about 4.5 mg/ml of gel, about 5 mg/ml of gel, or about 5.5 mg/ml of gel. In some embodiments, the visual additive is present in the gel at most about 1 mg/ml of gel, about 1.5 mg/ml of gel, about 2 mg/ml of gel, about 2.5 mg/ml of gel, about 3 mg/ml of gel, about 3.5 mg/ml of gel, about 4 mg/ml of gel, about 4.5 mg/ml of gel, about 5 mg/ml of gel, about 5.5 mg/ml of gel, or about 6 mg/ml of gel. Other examples of viscoelastic mediums treated for enhanced visualization are taught in WO2011084465, incorporated herein in its entirety.

In some embodiments, a further visualization additive is added to the gel, wherein the further visualization additive comprises a gas. In some embodiments, the gas is air, nitrogen, helium, oxygen, or any combination thereof. In some embodiments, the gas forms a plurality of bubbles within the spacer material. In some embodiments, a concentration of the further visualization additive in the spacer material is about 0.1% to about 15%. In some embodiments, the microbubbles have a size from about 1 μm to about 100 μm. In some embodiments, a concentration of the visualization additive in the spacer material is at least about 0.1%. In some embodiments, the gas is injected into the spacer material. In some embodiments, the visualization additive has an outer width of at least about 20 microns. In some embodiments, the gas is injected into the spacer material while the spacer material is under pressure. In some embodiments, the visualization additive has a diameter of at least about 20 microns. In some embodiments, the spacer material is agitated in an environment containing the gas to form the microbubbles. In some embodiments, the spacer material is pressurized and agitated in an environment containing the gas to form the microbubbles. In some embodiments, the microbubbles are formed before injection of the spacer material into a subject. In some embodiments, the microbubbles are formed during injection of the spacer material into a subject. In some embodiments, the microbubbles are formed during injection of the spacer material into a subject, wherein a geometry of needle forms the microbubbles. In some embodiments, the microbubbles are formed in-situ. In some embodiments, the cross-linked viscoelastic medium entraps and stabilizes the microbubbles. In some embodiments, the gels described herein comprise the further visualization additives, wherein the further visualization additives comprise a gas, without any other visualization additive.

In some embodiments, the viscoelastic medium is NASHA and the visualization additive is a known-imaging (e.g. MRI) contrast agent. In some embodiments, the visualization additive is any one of the compounds disclosed herein. In some embodiments, the visualization additive comprises a gadolinium-complex. In some embodiments, the gadolinium complex comprises gadopentetate dimeglumine. In some embodiments, a gel containing 5 mg/ml gadopentetate dimeglumine was prepared by weighing. In some embodiments, the visualization additive is mixed with the gel by manual stirring and the resulting gel was centrifuged to remove air bubbles one day prior to use. In some embodiments, the visualization additive is superparamagnetic iron oxide.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “radiative protection”, as used herein, refers to any type of volume augmentation of soft tissues, including, but not limited to, facial contouring (e.g. more pronounced cheeks or chin), correction of concave deformities (e.g. post-traumatic, HIV associated lipoatrophy) and correction of deep age-related facial folds. Thus, radiative protection may be used solely for cosmetic purposes or for medical purposes, such as following trauma or degenerative disease.

The term “degraded” implies that less than 20%, or less than 10%, of the medium remains in the body.

The term “soft tissue”, as used herein, refers to tissues that connect, support, or surround other structures and organs of the body. Soft tissue includes muscles, fibrous tissues and fat.

The terms “subepidermal administration” or “subcuticular administration”, as used herein, refer to administration beneath the epidermis of the skin, including administration into the dermis, subcutis or deeper, such as submuscularly or into the periosteum where applicable (in the vicinity of bone tissue.

As used herein, the term “therapeutic” involves any kind of preventive, alleviating or curative treatment.

By the term “super spacing,” as used herein, is meant the dictionary definition of the terms “super” and “spacing.” Along with the definition of spacing that is extraordinary relative to traditional documentation of similar tissue spacing in a similarly situated part of a given subject.

As used herein, a physiological solution or isotonic solution is a solution having an osmolarity in the range of about 200-about 400 mOsm/l, about 250-about 350 mOsm/l, or about 300 mOsm/l. For practical purposes, this osmolarity can be achieved by preparation of a 0.9% (0.154 M) NaCl solution.

The term “implant”, as used herein, refers widely to any type of implanted or implantable foreign object or material. Implants also include objects or materials that are nearly identical to non-foreign objects or materials. The implant is not limited to any particular shape. The final shape of the implant in the body is decided by the skilled man from the purpose of the treatment.

By the term “viscoelastic medium”, as used herein, is meant a medium that exhibits a combination of viscous and elastic properties. Specifically, the viscoelastic medium is injectable through a 20 gauge or larger needle, such as a 10-20 gauge needle, by application of a pressure of 15-50 N. In particular, the medium, or an implant or a medicament comprising the medium, is suitable for subepidermal injection into a human in need thereof at a desired site.

By the term “stabilized”, as used herein, is meant any form of chemical stabilization that, under physiological conditions, renders the stabilized compound more stable to biodegradation that the parent compound. Without being limited thereto, stabilized compounds include cross-linked compounds and partially cross-linked compounds.

By the term “derivative” of a polysaccharide, as used herein, is meant any suitable derivative thereof, including cross-linked polysaccharides and substituted polysaccharides, such as sulfated polysaccharides.

### Exemplary Embodiments

Among the exemplary embodiments are:
1. A composition comprising:
   a viscoelastic medium; and
   a first visual additive, wherein said first visual additive comprises a metal, and wherein said metal has a particle diameter of greater than or equal to 80 micrometers (μm).
2. The composition of claim 1, wherein said metal is a precious metal.
3. The composition of claim 1 or 2, wherein said metal or said precious metal is chosen from the group consisting of gold (Au), iodine (I), gadolinium (Gd), iron (Fe), barium (Ba), calcium (Ca), magnesium (Mg), and combinations thereof.
4. The composition of any one of claims 1 to 3, wherein said metal or precious metal is an isotope of gold (Au), iodine (I), gadolinium (Gd), iron (Fe), barium (Ba), calcium (Ca), magnesium (Mg), or combinations thereof.
5. The composition of any one of claims 1 to 4, wherein said metal or precious metal is a powder.
6. The composition of any one of claims 1 to 5, wherein said first visual additive has a radiographic density of between about 1.0 g/cm3 and 2.0 g/cm3.7. The composition of any one of claims 1 to 6, wherein half of said first visual additive is configured to disperse within a tissue within nine months.
8. The composition of any one of claims 1 to 7, wherein said particle diameter of said metal or precious metal is between about 80 μm and about 120 μm.
9. The composition of any one of claims 1 to 8, wherein said particle diameter of said metal or precious metal is about 100 μm.
10. The composition of any one of claims 1 to 9, wherein said first visual additive further comprises one or more microbubbles.
11. The composition of any one of claims 1 to 10, further comprising a second visual additive.
12. The composition of any one of claims 1 to 11, wherein said second visual additive is different than said first visual additive.
13. The composition of claim 11 or 12, wherein said second visual additive comprises one or more microbubbles.
14. The composition of any one of claims 1 to 13, wherein said first visual additive has a concentration within said viscoelastic medium of greater than 5 milligrams per milliliter (mg/ml).
15. The composition of any one of claims 1 to 14, wherein said first visual additive has a concentration within said viscoelastic medium of less than 90 mg/ml.
16. The composition of any one of claims 1 to 15, wherein said first visual additive has a concentration within said viscoelastic medium between 15 mg/ml and 30 mg/ml.
17. The composition of any one of claims 1 to 16, wherein said metal is between about 0.5 wt % and about 9.0 wt % of said composition.
18. The composition of any one of claims 1 to 17, wherein said metal or precious metal is between about 0.015 wt % and about 1.5 wt % of said composition.
19. The composition of any one of claims 1 to 18, wherein said viscoelastic medium comprises a volume of about 1 milliliter (ml) to about 50 ml.
20. The composition of any one of claims 1 to 19, wherein said composition is configured to be biodegradable.
21. The composition of any one of claims 1 to 20, wherein said composition is configured to be detectable on an imaging modality for at least 9 months.
22. The composition of any one of claims 1 to 21, wherein said composition is configured to not substantially migrate prior to or during imaging.
23. The composition of any one of claims 1 to 22, wherein said first visual additive is configured to not substantially migrate prior to or during imaging.
24. The composition of any one of claims 1 to 23, wherein said composition is configured to be disposed within a subject.
25. The composition of claim 24, wherein said subject is in need of radiography.
26. The composition of any one of claims 1 to 25, wherein said composition is configured to be disposed through injection.
27. The composition of either claim 25 or 26, wherein said composition is configured to be disposed subcutaneously or subepidermally.
28. The composition of any one of claims 1 to 27, wherein said composition is configured to be disposed within a tissue site.
29. The composition of claim 28, wherein said tissue site comprises one or more of, a fat tissue, a muscle tissue, an organ tissue, or a combination thereof.
30. The composition of any one of claims 1 to 29, wherein said composition is configured to be imaged on one or more modalities.
31. The composition of claim 30, wherein said one or more modalities comprise X-Ray, MRI, CT, CBCT, ultrasound, PET, SPECT or a combination thereof.
32. The composition of either claim 30 or 31, wherein said composition being configured to be imaged on said one or more modalities comprises said composition being configured to be imaged in real time on said one or more modalities.
33. The composition of any one of claims 1 to 32, wherein said composition is configured to be imaged within 30 min, within 90 min, within 4 hours, within 8 hours, or within 4 days of disposition.
34. The composition of any one of claims 1 to 33, wherein said viscoelastic medium comprises hyaluronic acid, polyethylene glycol, or dextranomers.
35. The composition of any one of claims 1 to 34, wherein said viscoelastic medium comprises hyaluronic acid, polyethylene glycol, or dextranomers at a concentration between about 5 mg/ml to about 100 mg/ml.
36. The composition of any one of claims 1 to 35, wherein said viscoelastic medium comprises gel particles at a size range of about 0.08 mm to about 5 mm.
37. The method of claim 36, wherein said gel particles comprise said metal particles.
38. The composition of any one of claims 1 to 37, wherein said viscoelastic medium comprises non-animal stabilized hyaluronic acid (“NASHA”).
39. The composition of any one of claims 1 to 38, wherein said viscoelastic medium expands within said tissue site to less than 10% of an original disposition volume.
40. The composition of any one of claims 1 to 39, wherein said viscoelastic medium is injected one time every six months.
41. The composition of any one of claims 1 to 40, wherein said viscoelastic medium is completely resorbed within 20 months.
42. The composition of any one of claims 1 to 41, wherein said viscoelastic medium is completely resorbed within 16 months.
43. The composition of any one of claims 1 to 42, wherein said viscoelastic medium is completely resorbed within 12 months.
44. The composition of any one of claims 1 to 43, wherein said first visual additive is present at an amount sufficient to generate a contrast to noise of about 0.1 to about 40 when the composition is scanned when using radiotherapy.
45. A method of visualizing a tissue, space or location of a radiographic subject, comprising disposing an imaging contrast composition, said composition comprising:
   a viscoelastic medium; and
   a first visual additive, wherein said first visual additive is a metal, and wherein said metal has a particle diameter greater than about 80 micrometers.
46. The method of claim 45, further comprising:
   (a) disposing the composition within a first tissue site, and
   (b) imaging said composition within said first tissue site.
47. The method of claim 46, wherein said metal has a particle diameter of greater than 100 micrometers.
48. A method of spacing a first tissue site of a subject from a second tissue site of said subject, the method comprising:
   (a) disposing a composition in a space between said first tissue site and said second tissue site, wherein said composition comprises a viscoelastic medium and a first visual additive, wherein said first visual additive is a metal, and wherein said metal has a particle diameter of greater than 80 micrometers (μm).
49. The method of any one of claims 45 to 48, wherein said first visual additive has a concentration within said viscoelastic medium, wherein the concentration results in a contrast to noise ratio of about 0.1 to about 40.
50. The method any one of claims 45 to 49, wherein said metal is a precious metal.
51. The method any one of claims 45 to 50, wherein said metal or said precious metal is chosen from the group consisting of gold (Au), iodine (I), gadolinium (Gd), iron (Fe), barium (Ba), calcium (Ca), magnesium (Mg), and combinations thereof.
52. The method of any one of claims 45 to 51, wherein said metal or precious metal is an isotope of gold (Au), iodine (I), gadolinium (Gd), iron (Fe), barium (Ba), calcium (Ca), magnesium (Mg), or combinations thereof.
53. The method of any one of claims 45 to 52, wherein said metal or precious metal is a powder.
54. The method of any one of claims 45 to 53, wherein said first visual additive has a radiographic density of between about 1.0 g/cm3 and 2.0 g/cm3.
55. The method of any one of claims 45 to 54, wherein half of said first visual additive is configured to disperse within a tissue within nine months.
56. The method of any one of claims 45 to 55, wherein said particle diameter of said metal or precious metal is between about 80 μm and about 120 μm.
57. The method of any one of claims 45 to 56, wherein said particle diameter of said metal or precious metal is about 100 μm.
58. The method of any one of claims 45 to 57, wherein said first visual additive further comprises one or more microbubbles.
59. The method of any one of claims 45 to 58, wherein the composition comprises a second visual additive.
60. The method of claim 59, wherein said second visual additive is different than said first visual additive.
61. The method of claim 59 or 60, wherein said second visual additive comprises one or more microbubbles.
62. The method of any one of claims 45 to 61, wherein said first visual additive has a concentration within said viscoelastic medium of greater than 5 milligrams per milliliter (mg/ml).
63. The method of any one of claims 45 to 62, wherein said first visual additive has a concentration within said viscoelastic medium of less than 90 mg/ml.
64. The method of any one of claims 45 to 63, wherein said first visual additive has a concentration within said viscoelastic medium between 15 mg/ml and 30 mg/ml.
65. The method of any one of claims 45 to 64, wherein said metal is between about 0.5 wt % and about 9.0 wt % of said composition.
66. The method of any one of claims 45 to 65, wherein said metal or precious metal is between about 0.015 wt % and about 1.5 wt % of said composition.
67. The method of any one of claims 45 to 66, wherein said viscoelastic medium comprises a volume of about 1 milliliter (ml) to about 50 ml.
68. The method of any one of claims 45 to 67, wherein said composition is configured to be biodegradable.
69. The method of any one of claims 45 to 68, wherein said composition is configured to be detectable on an imaging modality for at least 9 months.
70. The method of any one of claims 45 to 20, wherein said composition is configured to not substantially migrate prior to or during imaging, wherein the imaging occurs about 3 months to about 9 months the composition is disposed.
71. The method of any one of claims 45 to 70, wherein said first visual additive is configured to not substantially migrate prior to or during imaging, wherein the imaging occurs about 3 months to about 9 months the composition is disposed.
72. The method of any one of claims 45 to 71, wherein said composition is configured to be disposed within a subject.
73. The method of claim 72, wherein said subject is in need of radiography.
74. The method of any one of claims 45 to 73, wherein said composition is configured to be disposed through injection.
75. The method of any one of claims 45 to 74, wherein said composition is configured to be disposed subcutaneously or subepidermally.
76. The method of any one of claims 46 to 75, wherein said composition is configured to be disposed within said first tissue site.
77. The method of any one of claims 46 to 76, wherein said first tissue site comprises one or more of, a fat tissue, a muscle tissue, and organ tissue, or a combination thereof.
78. The method of any one of claims 45 to 77, wherein said composition is configured to be imaged on one or more modalities.
79. The method of claim 78, wherein said one or more modalities comprise X-Ray, MRI, CT, CBCT, ultrasound, PET, SPECT or a combination thereof.
80. The method of claim 78 or 79, wherein said composition being configured to be imaged on said one or more modalities comprises said composition being configured to be imaged in real-time on said one or more modalities.
81. The method of any one of claims 45 to 80, wherein said composition is configured to be imaged within 30 min, within 90 min, within 4 hours, within 8 hours, or within 4 days of disposition.
82. The method of any one of claims 45 to 81, wherein said viscoelastic medium comprises hyaluronic acid, polyethylene glycol, or dextranomers.
83. The method of any one of claims 45 to 82, wherein said viscoelastic medium comprises hyaluronic acid, polyethylene glycol, or dextranomers at a concentration between about 5 mg/ml to about 100 mg/ml.
84. The method of any one of claims 45 to 83, wherein said viscoelastic medium comprises gel particles at a size range of about 0.08 mm to about 5 mm.
85. The method of claim 84, wherein said gel particles comprise said metal particles.
86. The method of any one of claims 45 to 85, wherein said viscoelastic medium comprises non-animal stabilized hyaluronic acid (“NASHA”).
87. The method of any one of claims 45 to 86, wherein said viscoelastic medium expands within said first tissue site to less than 10% of an original disposition volume.
88. The method of any one of claims 45 to 87, wherein said viscoelastic medium is injected one time every six months.
89. The method of any one of claims 45 to 88, wherein said viscoelastic medium is completely resorbed within 20 months.
90. The method of any one of claims 45 to 89, wherein said viscoelastic medium is completely resorbed within 16 months.
91. The method of any one of claims 45 to 90, wherein said viscoelastic medium is completely resorbed within 12 months.
92. A method of treating cancer in a subject in need thereof, the method comprising: identifying a tumor within the subject;
   administering a composition adjacent to the tumor, the composition comprising:
      a viscoelastic medium; and
      a first visual additive, wherein said first visual additive is a metal, and wherein said metal has a particle diameter greater than about 80 micrometers; and
   applying a dose of radiation to the tumor.
93. The method of claim 92, wherein said first visual additive has a concentration within said viscoelastic medium, wherein the concentration results in a contrast to noise ratio of about 0.1 to about 40.
94. The method of claim 92 or 93, wherein said metal is a precious metal.
95. The method any one of claims 92 to 94, wherein said metal or said precious metal is chosen from the group consisting of gold (Au), iodine (I), gadolinium (Gd), iron (Fe), barium (Ba), calcium (Ca), magnesium (Mg), and combinations thereof.
96. The method of any one of claims 92 to 95, wherein said metal or precious metal is an isotope of gold (Au), iodine (I), gadolinium (Gd), iron (Fe), barium (Ba), calcium (Ca), magnesium (Mg), or combinations thereof.
97. The method of any one of claims 92 to 96, wherein said metal or precious metal is a powder.
98. The method of any one of claims 92 to 97, wherein said first visual additive has a radiographic density of between about 1.0 g/cm3 and 2.0 g/cm3.
99. The method of any one of claims 92 to 98, wherein half of said first visual additive is configured to disperse within a tissue within nine months.
100. The method of any one of claims 92 to 99, wherein said particle diameter of said metal or precious metal is between about 80 μm and about 120 μm.
101. The method of any one of claims 92 to 100, wherein said particle diameter of said metal or precious metal is about 100 μm.
102. The method of any one of claims 92 to 101, wherein said first visual additive further comprises one or more microbubbles.
103. The method of any one of claims 92 to 102, wherein the composition comprises a second visual additive.
104. The method of claim 103, wherein said second visual additive is different than said first visual additive.
105. The method of claim 103 or 104, wherein said second visual additive comprises one or more microbubbles.
106. The method of any one of claims 92 to 105, wherein said first visual additive has a concentration within said viscoelastic medium of greater than 5 milligrams per milliliter (mg/ml).
107. The method of any one of claims 92 to 106, wherein said first visual additive has a concentration within said viscoelastic medium of less than 90 mg/ml.
108. The method of any one of claims 92 to 107, wherein said first visual additive has a concentration within said viscoelastic medium between 15 mg/ml and 30 mg/ml.
109. The method of any one of claims 92 to 108, wherein said metal is between about 0.5 wt % and about 9.0 wt % of said composition.
110. The method of any one of claims 92 to 109, wherein said metal or precious metal is between about 0.015 wt % and about 1.5 wt % of said composition.
111. The method of any one of claims 92 to 110, wherein said viscoelastic medium comprises a volume of about 1 milliliter (ml) to about 50 ml.
112. The method of any one of claims 92 to 111, wherein said composition is configured to be biodegradable.
113. The method of any one of claims 92 to 112, wherein said composition is configured to be detectable on an imaging modality for at least 9 months.
114. The method of any one of claims 92 to 113, wherein said composition is configured to not substantially migrate prior to or during imaging, wherein the imaging occurs about 3 months to about 9 months the composition is disposed.
115. The method of any one of claims 92 to 114, wherein said first visual additive is configured to not substantially migrate prior to or during imaging, wherein the imaging occurs about 3 months to about 9 months the composition is disposed.
116. The method of any one of claims 92 to 115, wherein said composition is configured to be disposed within the subject.
117. The method of claim 116, wherein the subject is in need of radiography.
118. The method of any one of claims 92 to 117, wherein said composition is configured to be disposed through injection.
119. The method of any one of claims 92 to 118, wherein said composition is configured to be disposed subcutaneously or subepidermally.
120. The method of any one of claims 93 to 119, wherein said composition is configured to be disposed within said first tissue site.
121. The method of any one of claims 93 to 120, wherein said first tissue site comprises one or more of, a fat tissue, a muscle tissue, and organ tissue, or a combination thereof.
122. The method of any one of claims 92 to 121, wherein said composition is configured to be imaged on one or more modalities.
123. The method of claim 122, wherein said one or more modalities comprise X-Ray, MRI, CT, CBCT, ultrasound, PET, SPECT or a combination thereof.
124. The method of claim 122 or 123, wherein said composition being configured to be imaged on said one or more modalities comprises said composition being configured to be imaged in real-time on said one or more modalities.
125. The method of any one of claims 92 to 124, wherein said composition is configured to be imaged within 30 min, within 90 min, within 4 hours, within 8 hours, or within 4 days of disposition.
126. The method of any one of claims 92 to 125, wherein said viscoelastic medium comprises hyaluronic acid, polyethylene glycol, or dextranomers.
127. The method of any one of claims 92 to 126, wherein said viscoelastic medium comprises hyaluronic acid, polyethylene glycol, or dextranomers at a concentration between about 5 mg/ml to about 100 mg/ml.
128. The method of any one of claims 92 to 127, wherein said viscoelastic medium comprises gel particles at a size range of about 0.08 mm to about 5 mm.
129. The method of claim 128, wherein said gel particles comprise said metal particles.
130. The method of any one of claims 92 to 129, wherein said viscoelastic medium comprises non-animal stabilized hyaluronic acid (“NASHA”).
131. The method of any one of claims 92 to 130, wherein said viscoelastic medium expands within said first tissue site to less than 10% of an original disposition volume.
132. The method of any one of claims 92 to 131, wherein said viscoelastic medium is injected one time every six months.
133. The method of any one of claims 92 to 132, wherein said viscoelastic medium is completely resorbed within 20 months.
134. The method of any one of claims 92 to 133, wherein said viscoelastic medium is completely resorbed within 16 months.
135. The method of any one of claims 92 to 134, wherein said viscoelastic medium is completely resorbed within 12 months.

### Examples

The following illustrative examples are representative of embodiments of the software applications, systems, and methods described herein and are not meant to be limiting in any way.

### Example 1—Spacer Injection Between the Head of Pancreas and the Duodenum

In one example, an absorbable hydrogel comprising hyaluronic acid microparticles was injected in the space between the Head of Pancreas (HOP) and the third portion of the duodenal loop using an 18-gauge needle. An endoscopic ultrasound (EUS) coupled to an ultrasound workstation was used to identify the duodenum and HOP interface, followed by hydrogel injection in this peripancreatic space using a 19-gauge fine needle aspiration needle in increments of 1 mL until the desired space was generated. The EUS scope was then adjusted (slightly advanced or retracted) around the target region to provide shape and conformity around the tumor to generate the desired space, with the total injection volume ranging from 1.0 mL to 27 mL.

A visible separation between the HOP and duodenum was created to confirm the location of the hydrogel and to measure the distance created between the duodenum and HOP. The mean distance of separation by hydrogel placement was measured by averaging the measured thickness of the gel on each CT slice on which gel was visualized on the post injection simulation CT scan obtained with a 2-mm slice thickness. The mean thickness of the spacer was 1.1 cm (0.9 cm-1.2 cm) and 0.9 cm (0.8 cm-1.1 cm) for EUS cadaveric specimens 1 and 2 on the post injection CT scans, respectively.

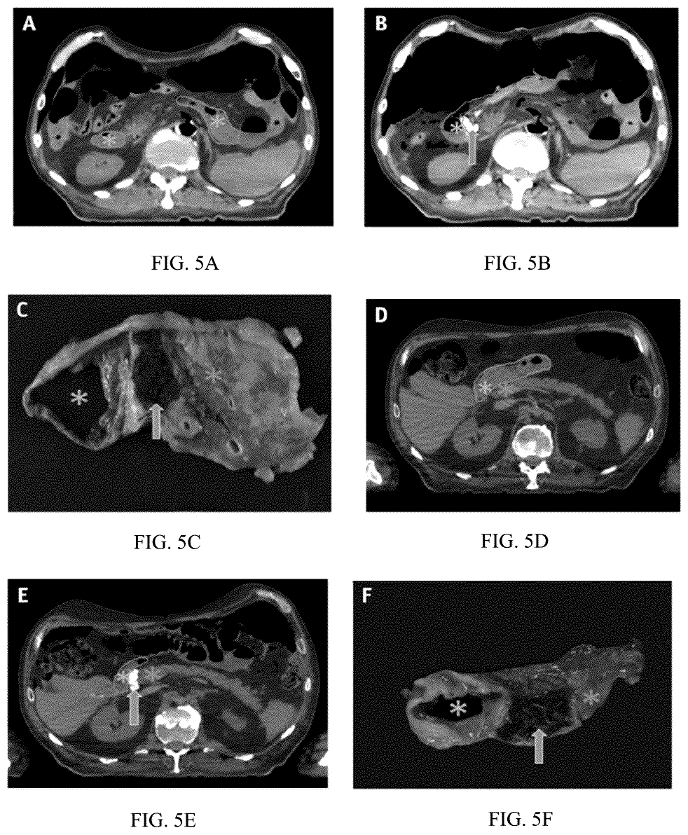

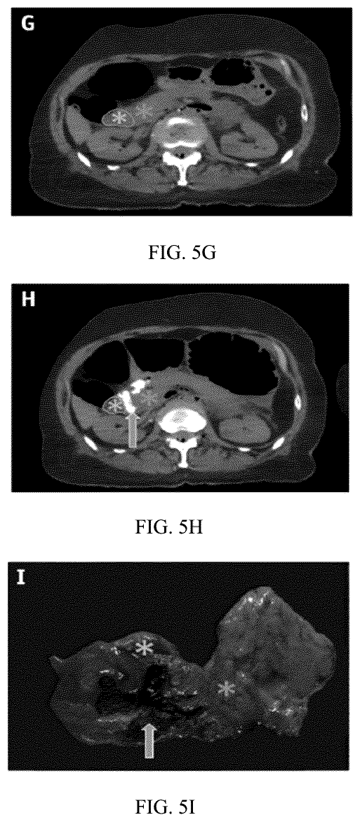

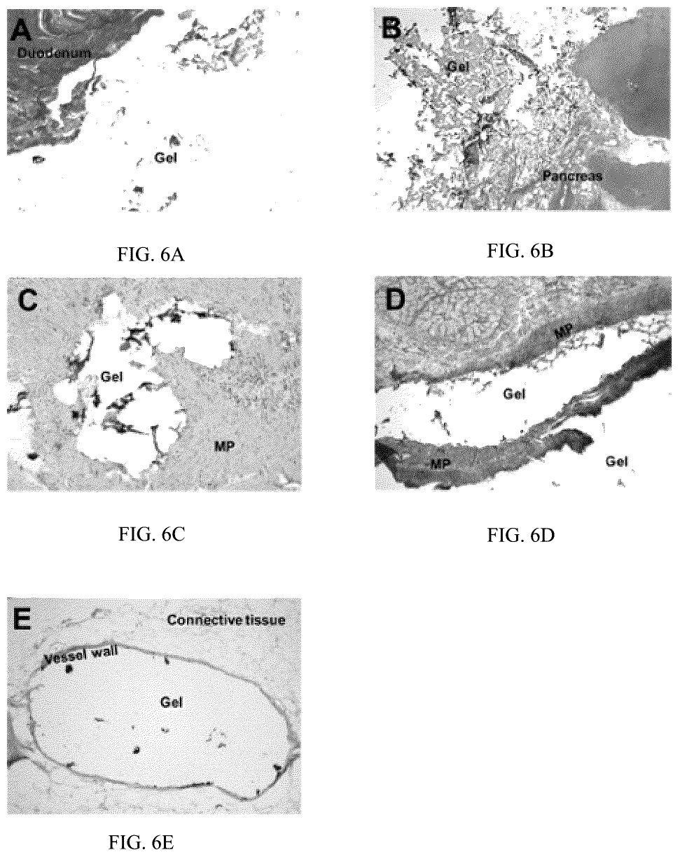

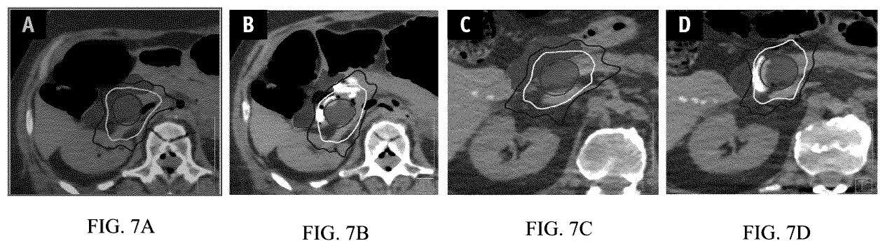

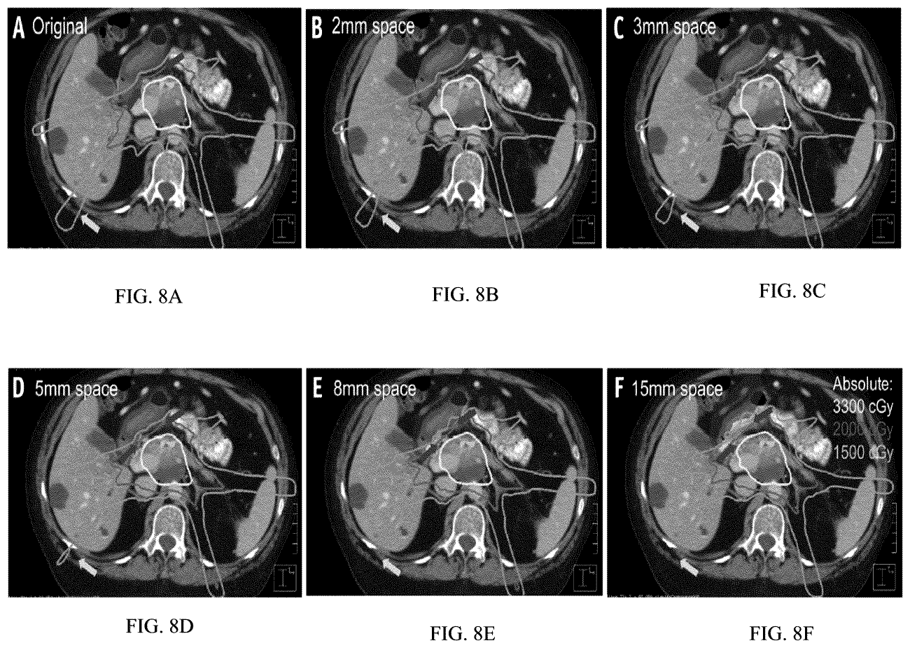

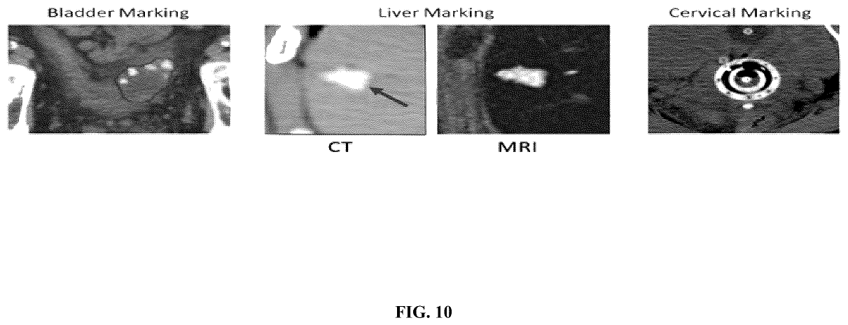

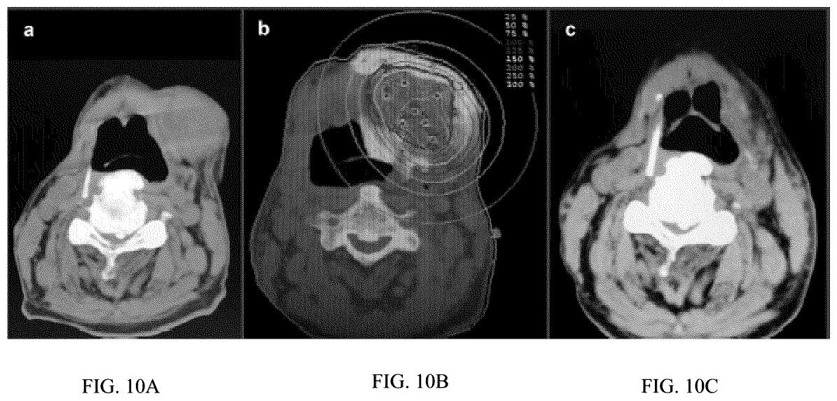

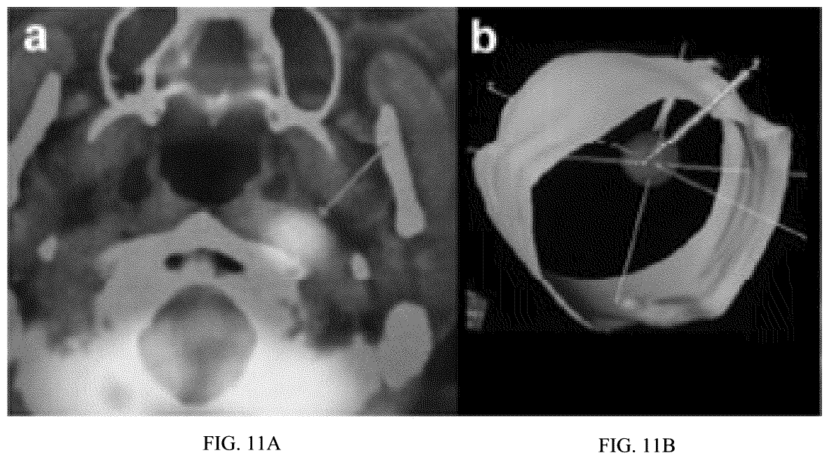

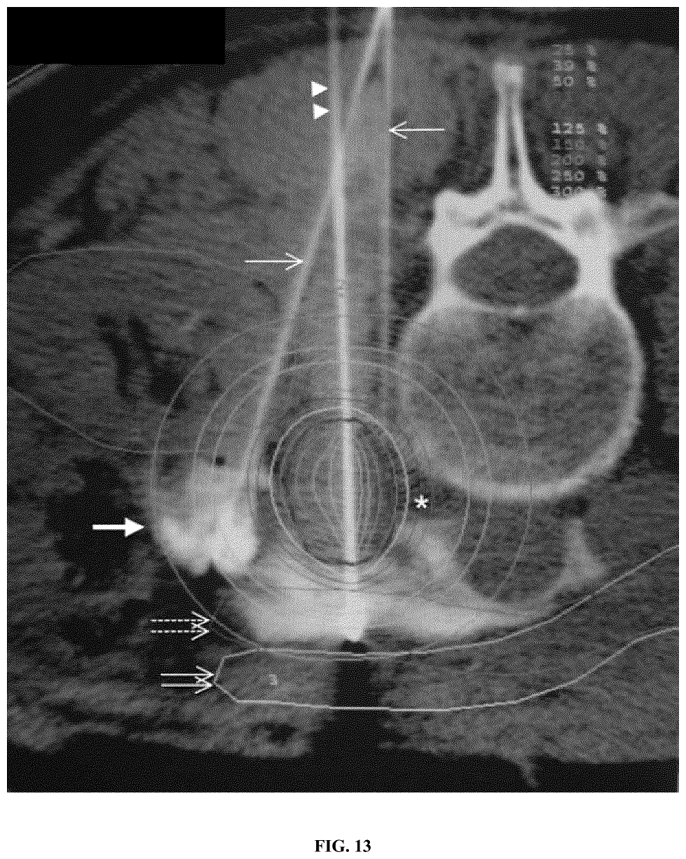

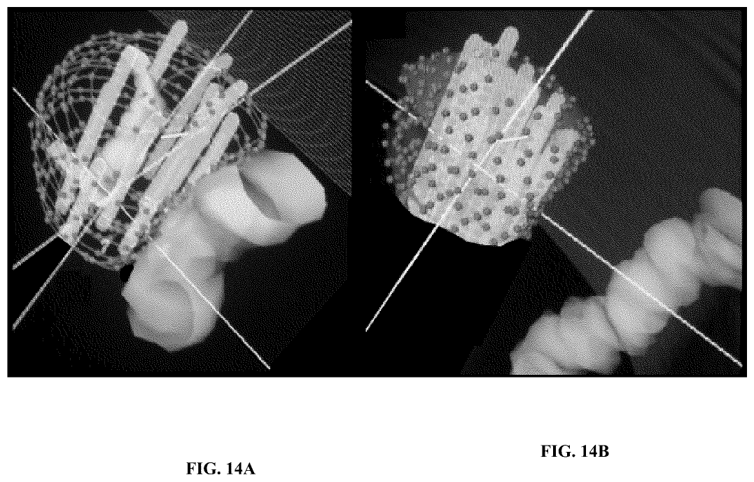

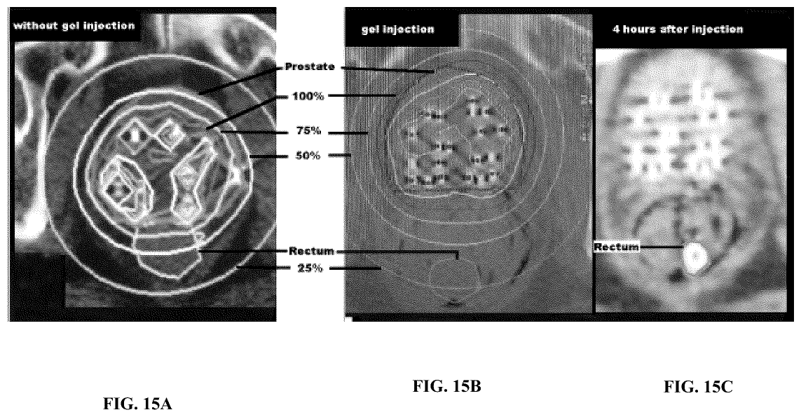

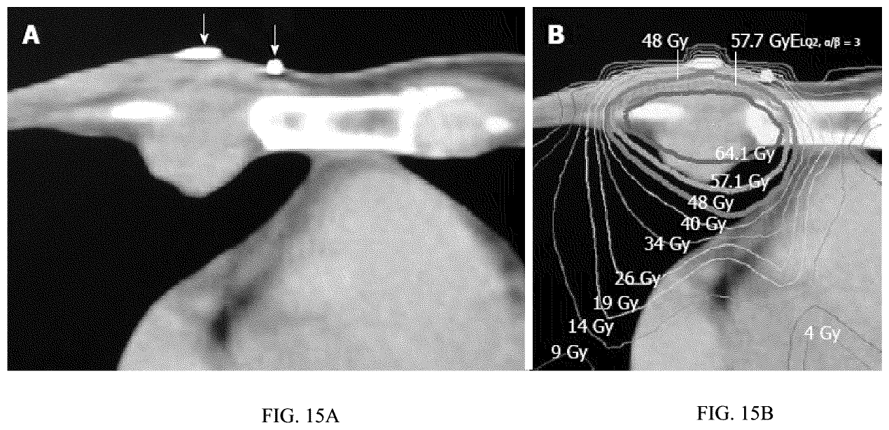

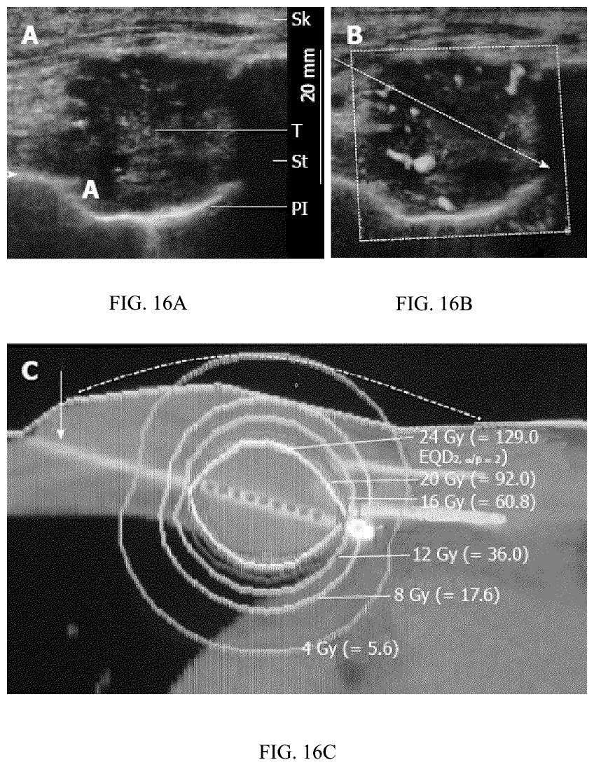

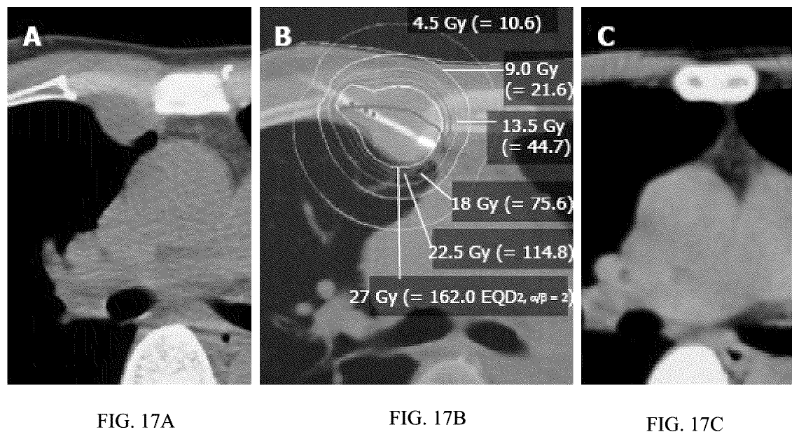

FIG. 5A is an exemplary image of a computed tomography scan before hydrogel spacer injection between the head of the pancreas and duodenum. FIG. 5B is an exemplary image of a computed tomography scan after hydrogel spacer injection between the head of the pancreas and duodenum. FIG. 5C is an exemplary image of a gross histologic specimen after hydrogel spacer injection between the head of the pancreas and duodenum. FIG. 5D is an exemplary image of a computed tomography scan before a laparotomy hydrogel spacer injection between the head of the pancreas and duodenum. FIG. 5E is an exemplary image of a computed tomography scan after a laparotomy hydrogel spacer injection between the head of the pancreas and duodenum. FIG. 5F is an exemplary image of a gross histologic specimen after a laparotomy hydrogel spacer injection between the head of the pancreas and duodenum. FIG. 5G is an exemplary image of a computed tomography scan before an endoscopically hydrogel spacer injection between the head of the pancreas and duodenum. FIG. 5H is an exemplary image of a computed tomography scan after an endoscopically hydrogel spacer injection between the head of the pancreas and duodenum. FIG. 5I is an exemplary image of a gross histologic specimen after an endoscopically hydrogel spacer injection between the head of the pancreas and duodenum. FIG. 6A is a first exemplary image of a formalin-fixed, paraffin-embedded section after hematoxylin and eosin staining. FIG. 6B is a second exemplary image of a formalin-fixed, paraffin-embedded section after hematoxylin and eosin staining. FIG. 6C is a first exemplary high magnification image of a formalin-fixed, paraffin-embedded section after hematoxylin and eosin staining. FIG. 6D is a third exemplary image of a formalin-fixed, paraffin-embedded section after hematoxylin and eosin staining. FIG. 6E is a second exemplary high magnification image of a formalin-fixed, paraffin-embedded section after hematoxylin and eosin staining. FIG. 7A is a first exemplary stereotactic body radiation therapy plan before hydrogel spacer placement. FIG. 7B is a first exemplary stereotactic body radiation therapy plan after hydrogel spacer placement. FIG. 7C is a second exemplary stereotactic body radiation therapy plan before hydrogel spacer placement. FIG. 7D is a second exemplary stereotactic body radiation therapy plan after hydrogel spacer placement. FIG. 8A is an exemplary baseline image of a computed tomography scan and stereotactic body radiation therapy plan of the duodenum. FIG. 8B is an exemplary image of a computed tomography scan and stereotactic body radiation therapy plan of the duodenum with a 2 mm spacing. FIG. 8C is an exemplary image of a computed tomography scan and stereotactic body radiation therapy plan of the duodenum with a 3 mm spacing. FIG. 8D is an exemplary image of a computed tomography scan and stereotactic body radiation therapy plan of the duodenum with a 5 mm spacing. FIG. 8E is an exemplary image of a computed tomography scan and stereotactic body radiation therapy plan of the duodenum with a 8 mm spacing. FIG. 8F is an exemplary image of a computed tomography scan and stereotactic body radiation therapy plan of the duodenum with a 15 mm spacing.

### Example 2—Subcutaneous Breast Spacer Injection

In one example, ultrasound-guided spacer injections of iodined polyethylene glycol (PEG) were performed to form a spacer thickness of greater than 5 mm. Pre and post-injection CT scans were used after defining a clinical target volume. Maximum dose to small skin volumes (D_{0.2 cc}) and existence of hotspots (isodose ≥90% on 1 cm2 of skin) were calculated as skin toxicity indicators. After removal of the breast, the spacer was injected directly under the skin to create a 5 mm extra space between skin and the superficial fascial layer of the breast by hydrodissection.

Intervention success was 90.9%. Hydrodissection was feasible in 63.6% of cases. Median system usability scale score was 82.5 for PEG (p<0.001). Mean Dwas 80.8 Gy without spacer and 53.7 Gy with spacer (p<0.001). Skin hotspots were present in 40.9% without spacer but none with spacer (p<0.001).C

Success percentages of the spacer injection were high (91%) with a marker.

### Example 3—Subcutaneous Esophageal Spacer Injection

In one example, a gel was prepared as a viscous mixture of 10 ml of 1 mg/ml hyaluronic acid (HA), 0.8 ml of contrast media consisting of 300 mg iodine/ml. Following local anesthesia of subcutaneous tissue with lidocaine and under ultrasound and CT guidance, a 21-gauge needle was inserted at a puncture point lateral to the trachea at the level of the upper edge of the sternum and advanced to the location for gel injection. The needle penetrated the skin first; second, subcutaneous tissue (superficial cervical fascia, SCF) between both edges of the platysma in SCF; third, the relatively hard investing layer of deep cervical fascia (DCF) containing the suprasternal space (space of Burns) filled with adipose connective tissue and the transverse cervical vein, and adipose tissue below this fascia; and fourth, the pretracheal layer of DCF over the peritracheal space continuous with paraoesophageal adipose tissue. To advance the needle safely, the trachea was shifted by about 5-10 mm manually to the right or left when necessary. Using this route, the needle passes medial to the sternohyoid and stemothyroid strap muscles to avoid the influence of muscle contraction on the needle. When the needle tip reached the predetermined injection point, the gel was injected to create a space, forcing the esophagus away from the target. The created space was confirmed by CT.

### Example 4—Reduction of Dose of Radiotherapy to Tissue Proximate to the Site of Radiotherapy to Pancreatic Cancer

Two patients are treated for pancreatic cancer by radiotherapy. One patient receives a PEG-based spacer injected using the technique described in Example 1. The injection technique from Example 1 is utilized to create a space between the site of radiotherapy and the duodenum between the range of 0.9 cm-1.2 cm. The dose of radiation in the tissue proximate to the site of radiotherapy is reduced by 40% in the patient whom received the spacer relative to the patient whom did not receive the spacer.

### Example 5—Reduction of Dose of Radiotherapy to Tissue Proximate to the Site of Radiotherapy to Esophageal Cancer

Two patients are treated for esophageal cancer by radiotherapy. One patient receives a PEG-based spacer injected using the technique described in Example 1. The injection technique from Example 4 is utilized to create a space between the site of radiotherapy and the duodenum between the range of 0.9 cm-1.2 cm. The dose of radiation in the tissue proximate to the site of radiotherapy is reduced by 20% in the patient whom received the spacer relative to the patient whom did not receive the spacer.

### Example 6—Erasable Hyaluronic Acid Spacer

The techniques described in Examples 4 and 5 are utilized. The patient is in rapid need of a reduction in the size of the spacer following radiotherapy due to the pressure exerted by the spacer onto the patient's esophagus. The caring physician injects a volume of about 1 ml to about 10 ml of hyaluronidase at about 1 U to about 100 U until the desired volume of the spacer is achieved.

### Example 7—Erasable PEG-Based Spacer

The techniques described in Examples 4 and 5 are utilized. The patient is in rapid need of a reduction in the size of the spacer following radiotherapy due to the pressure exerted by the spacer onto the patient's esophagus. The caring physician injects a volume of about 1 ml to about 50 ml of water until the desired volume of the spacer is achieved.

### Example 8—Shrinking Hyaluronic Acid Super Spacer

The injection technique of Example 4 is adapted to accommodate for a dual syringe. One syringe contains the desired volume of cross-linked hyaluronic acid particles while the opposite syringe contains a degradable nanoparticles containing hyaluronidase that are soluble in hyaluronic acid. The concentration of degradable nanoparticles contained in the syringe is determined by the determined length of radiotherapy. A larger spacing distance relative to the use of spacers without degradable nanoparticles containing hyaluronidase is achieved because the spacer shrinks before tissue damage due to great displacement of said tissue occurs or before undesired cosmetic changes to the anatomy of the subject occurs. This outcome is only made possible by the shrinking super spacer.

### Example 9—Use of Shrinking Hyaluronic Acid Super Spacer on a Subject Suffering from Melanoma on the Scalp

A subject has a melanoma tumor located his scalp. The tumor measures 0.4 cm×0.8 cm×0.2 cm. Radiotherapy is selected for as the desired treatment. The treating physician determines that the radiotherapy will require about 10 minutes. The treating physician desires to use a dose of radiotherapy that is greater than what is conventional due to the state of the tumor. The injection technique described in Examples 4 and 7 are adapted to a subcutaneous injection on the scalp. A volume between about 3 ml to about 6 ml is injected to space the tumor from the subject's brain. A specific proportion of this volume consists of the degradable nanoparticles containing hyaluronidase. This spaces the brain from the tumor by a distance that damages the connective tissue proximate to the skull and creates undesired cosmetic appearance. However, as soon as the spacer is injected in the space between the brain and the tumor, the spacer begins to shrink. Radiotherapy is performed and the subject's brain is exposed to about 20% less dose of radiotherapy compared to a subject that did not receive the spacer. The radiotherapy session concludes and the subject's scalp looks like the scalp of the subject that did not receive the spacer because the degradable nanoparticles have degraded the spacer.

### Example 10—Shrinking PEG-Based Super Spacer

The injection technique of Example 4 is adapted to accommodate for a dual syringe. One syringe contains the desired volume of PEG particles while the opposite syringe contains a degradable nanoparticles containing water that are soluble in hyaluronic acid. The concentration of degradable nanoparticles contained in the syringe is determined by the determined length of radiotherapy. A larger spacing distance relative to the use of spacers without degradable nanoparticles containing water is achieved because the spacer shrinks before tissue damage due to great displacement of said tissue occurs or before undesired cosmetic changes to the anatomy of the subject occurs. This outcome is only made possible by the shrinking super spacer.

### Example 11—Use of Shrinking PEG-Based Super Spacer on a Subject Suffering from Melanoma on the Scalp

A subject has a melanoma tumor located his scalp. The tumor measures 0.4 cm×0.8 cm×0.2 cm. Radiotherapy is selected for as the desired treatment. The treating physician determines that the radiotherapy will require about 10 minutes. The treating physician desires to use a dose of radiotherapy that is greater than what is conventional due to the state of the tumor. The injection technique described in Examples 4 and 7 are adapted to a subcutaneous injection on the scalp. A volume between about 3 ml to about 6 ml is injected to space the tumor from the subject's brain. A specific proportion of this volume consists of the degradable nanoparticles containing water. This spaces the brain from the tumor by a distance that damages the connective tissue proximate to the skull and creates a undesired cosmetic appearance. However, as soon as the spacer is injected in the space between the brain and the tumor, the spacer begins to shrink. Radiotherapy is performed and the subject's brain is exposed to about 20% less dose of radiotherapy compared to a subject that did not receive the spacer. The radiotherapy session concludes and the subject's scalp looks like the scalp of the subject that did not receive the spacer because the degradable nanoparticles have degraded the spacer.

### Example 12—Use of Improved Tissue Spacers for Interventional Oncology

7.4 g of poloxamer 407 and 0.2 g of polyethylene glycol-modified polylactic acid PLA-PEG, are added to 20 ml and 10 ml of pure water, and placed in a 25° C. for 3 days to completely dissolve the polymer. Then mixing the two and vortexing, results in a gel dispersion that is distilled off under reduced pressure and water. The spacer is then dried and sealed, and stored at 4° C. storage.

The hydrogel described above is inserted directly into a blood vessel that is directly couple to a tumor. The cancer is treated by the hydrogel blocking tumor blood supply, the tumor becomes ischemic, hypoxic and necrotic. Further, the cancer tissue necrosis continues to stimulate the body's immune system, it is possible to remove distant metastases (preferably in melanoma); delivering embolic agent(s) into the tumor with a chemotherapeutic agent mixed target artery feeding, both to block the blood supply, but also slows the release of chemotherapy drugs play a role in local chemotherapy, therefore, short-term efficacy of oncolytic outcomes.

### Example 13—Iodine Incorporation into PEG

A PEG SG (with an SG count of 2.3 per molecule) containing an iodine core was synthesized. The PEG-I molecule was 6400 Daltons, of which iodine was 381 Daltons (5.9%). Thus, for example, with this iodine content, the percent solids of PEG-I in hydrogel that resulted in 0.10% and 0.2% iodine concentration in the resultant matrix was 1.68 and 3.36%. Table II of WO2011084465, incorporated herein in its entirety, shows how PEG-I concentrations can be manipulated to obtain a percentage iodine content, which in turn can be related to a CT number.

### Example 14—Preparing a Gadopentetate Dimeglumine with NASHA-Gel

A gel containing 5 mg/ml gadopentetate dimeglumine was prepared by weighing the gadopentetate dimeglumine (Magnevist 469 mg/ml (0.5 mmol Gd/ml, Shering)) and thoroughly mixing the gadopentetate dimeglumine with the NASHA-gel (20 mg HA/ml, Q-Med) by manual stirring. The resulting gel was centrifuged to remove air bubbles. The particles of the resulting gel had an average size of 1 mm.

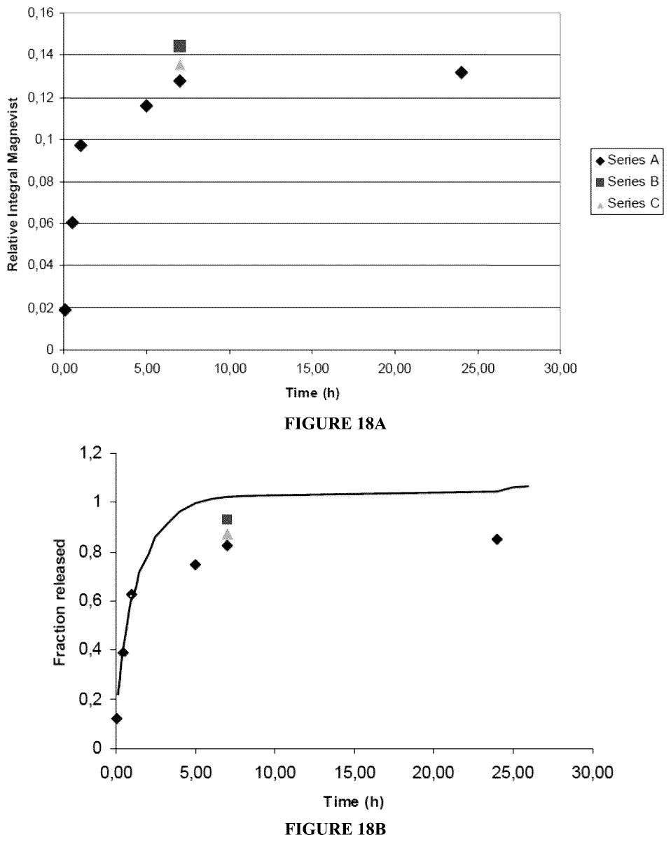

The release of the gadolinium complexes from the gels was measured using a USP-paddle system. The release of the gadolinium complex was followed using NMR and ICP-MS. A 2D spin echo (SE) sequence and a 3D gradient Echo (FFE) sequence were made, both sequences were made with and without fat saturation (FS). MRI was also made on gels that have released the gadolinium complexes for different periods of time. The initial release of the gadolinium complex was relatively fast and the rate corresponds quite well with the diffusion rate of small drug molecules (FIG. 18A). The ICP-MS analysis after 7 and 24 hours release showed that 82 and 85% of the gadolinium has been released (FIG. 18B), indicating that a small amount of gadolinium interacts and was released at a much slower rate. In the MRI measurements on the gel, at the first time points (30 minutes and 90 minutes) the NASHA-gel gave a strong contrast compared to water and oil. At the later times (8 hours up to 4 days) the contrast was much weaker but still visible compared to NASHA-gel without the gadolinium complexes showing that a small amount of gadolinium seems to interact with the NASHA-gel. However, the amount is not enough to give a contrast useful for in vivo MRI. The strength of the signal in the MRI measurements is summarized in Table 1 below.

TABLE 1
Strength of the signal in the MRI measurements

| | | T1W/SE | T1W/FFE/ | T1W/FFE/ |
|---|---|---|---|---|
| | T1W/SE | FS | 3D | 3D FS |
| Air | 18.00 | 17.00 | 3.00 | 3.00 |
| Water at left | 337.00 | 283.00 | 116.00 | 133.00 |
| reference | | | | |
| Water at right | 366.00 | 316.00 | 133.00 | 153.00 |
| reference | | | | |
| Oil | 880.00 | 290.00 | 355.00 | 91.00 |
| Positive reference | 1648.00 | 1588.00 | 1951.00 | 1901.00 |
| 30 min | 1746.00 | 1726.00 | 1790.00 | 1926.00 |
| 90 min | 1980.00 | 1990.00 | 1439.00 | 1761.00 |
| 8 hours | 858.00 | 830.00 | 319.00 | 449.00 |
| 1 day | 609.00 | 636.00 | 217.00 | 348.00 |
| 2 days | 665.00 | 655.00 | 247.00 | 351.00 |
| 4 days | 865.00 | 798.00 | 344.00 | 401.00 |
| Negative | 367.00 | 326.00 | 137.00 | 152.00 |
| reference | | | | |

### Example 15—Creating an Omni-Opaque Spacer Using any of the Materials Described Herein

A viscoelastic medium comprising any one or a combination of the materials described herein is developed into a particle size configured to be drawn into syringe. A fine (20 gauge or greater) needle is attached to the syringe wherein a portion of the interior surface of the needle contains a mesh structure. This mesh structure is configured to generate fairly homogenous microbubbles within the viscoelastic medium that is pushed through the mesh.

MRI is made on gels comprising the viscoelastic medium with microbubbles for different periods of time. The initial visualization of the gels with microbubbles corresponds relatively well with the other gels combined with radiopaque agents as described herein. At the time points 30 minutes and 90 minutes, the gel with microbubbles gives a strong contrast compared to water and oil, comparable to the gels with radiopaque agents described herein.

At the later times (8 hours up to 4 days) the contrast of the gel with the microbubbles retains enough to give a contrast useful for in vivo MRI, CT, ultrasound, or any other imaging modality known in the art are administered to the various gels described herein comprising any one of the radiopaque agents described herein, including microbubbles, or a combination thereof. These gels retain contrast sufficient for in vivo imaging at real-time, 30 minutes, 90 minutes, and up to 8 hours and up to 4 days.

### Example 16—Visualizing a Cavity

In another example, a dermal incision was made with a scalpel at the desired site and a viscoelastic medium from Example 15 was administered by injection under the epidermis, wherein microbubbles are formed within the spacing material as it passes through the mesh structure of the needle into the injection site. The mesh structure comprises spaces of about 0.001 mm² to about 1.5 mm². The mesh structure comprises spaces of about 0.001 mm² to about 0.005 mm², about 0.001 mm² to about 0.01 mm², about 0.001 mm² to about 0.05 mm², about 0.001 mm² to about 0.06 mm², about 0.001 mm² to about 0.07 mm², about 0.001 mm² to about 0.08 mm², about 0.001 mm² to about 0.09 mm², about 0.001 mm² to about 0.1 mm², about 0.001 mm² to about 0.5 mm², about 0.001 mm² to about 1 mm², about 0.001 mm² to about 1.5 mm², about 0.005 mm² to about 0.01 mm², about 0.005 mm² to about 0.05 mm², about 0.005 mm² to about 0.06 mm², about 0.005 mm² to about 0.07 mm², about 0.005 mm² to about 0.08 mm², about 0.005 mm² to about 0.09 mm², about 0.005 mm² to about 0.1 mm², about 0.005 mm² to about 0.5 mm², about 0.005 mm² to about 1 mm², about 0.005 mm² to about 1.5 mm², about 0.01 mm² to about 0.05 mm², about 0.01 mm² to about 0.06 mm², about 0.01 mm² to about 0.07 mm², about 0.01 mm² to about 0.08 mm², about 0.01 mm² to about 0.09 mm², about 0.01 mm² to about 0.1 mm², about 0.01 mm² to about 0.5 mm², about 0.01 mm² to about 1 mm², about 0.01 mm² to about 1.5 mm², about 0.05 mm² to about 0.06 mm², about 0.05 mm² to about 0.07 mm², about 0.05 mm² to about 0.08 mm², about 0.05 mm² to about 0.09 mm², about 0.05 mm² to about 0.1 mm², about 0.05 mm² to about 0.5 mm², about 0.05 mm² to about 1 mm², about 0.05 mm² to about 1.5 mm², about 0.06 mm² to about 0.07 mm², about 0.06 mm² to about 0.08 mm², about 0.06 mm² to about 0.09 mm², about 0.06 mm² to about 0.1 mm², about 0.06 mm² to about 0.5 mm², about 0.06 mm² to about 1 mm², about 0.06 mm² to about 1.5 mm², about 0.07 mm² to about 0.08 mm², about 0.07 mm² to about 0.09 mm², about 0.07 mm² to about 0.1 mm², about 0.07 mm² to about 0.5 mm², about 0.07 mm² to about 1 mm², about 0.07 mm² to about 1.5 mm², about 0.08 mm² to about 0.09 mm², about 0.08 mm² to about 0.1 mm², about 0.08 mm² to about 0.5 mm², about 0.08 mm² to about 1 mm², about 0.08 mm² to about 1.5 mm², about 0.09 mm² to about 0.1 mm², about 0.09 mm² to about 0.5 mm², about 0.09 mm² to about 1 mm², about 0.09 mm² to about 1.5 mm², about 0.1 mm² to about 0.5 mm², about 0.1 mm² to about 1 mm², about 0.1 mm² to about 1.5 mm², about 0.5 mm² to about 1 mm², about 0.5 mm² to about 1.5 mm², or about 1 mm² to about 1.5 mm². The mesh structure comprises spaces of about 0.001 mm², about 0.005 mm², about 0.01 mm², about 0.05 mm², about 0.06 mm², about 0.07 mm², about 0.08 mm², about 0.09 mm², about 0.1 mm², about 0.5 mm², about 1 mm², or about 1.5 mm². The mesh structure comprises spaces of at least about 0.001 mm², about 0.005 mm², about 0.01 mm², about 0.05 mm², about 0.06 mm², about 0.07 mm², about 0.08 mm², about 0.09 mm², about 0.1 mm², about 0.5 mm², or about 1 mm². The mesh structure comprises spaces of at most about 0.005 mm², about 0.01 mm², about 0.05 mm², about 0.06 mm², about 0.07 mm² about 0.08 mm², about 0.09 mm², about 0.1 mm², about 0.5 mm², about 1 mm², or about 1.5 mm². The viscoelastic medium is injected into a treatment site of a patient wherein the viscoelastic medium comprises homogenous microbubbles. Thereafter, an MRI, CT, ultrasound, or any combination thereof is performed on the treatment site to determine that a cavity is formed by the spacer material between a treatment organ and a proximal tissue location. The gel is configured to be visualized at real-time, 30 minutes, 90 minutes, and up to 8 hours and up to 4 days post-injection. The microbubbles configure the gel to be visible enough for in vivo imaging among all modalities, for up to 4 days.

### Example 17—Preparing a Gadopentetate Dimeglumine with NASHA-Gel

A gel containing more than 5 mg/ml, more than 6 mg/ml, more than 7 mg/ml, more than 8 mg/ml, more than 9 mg/ml, or more than 10 mg/ml gadopentetate dimeglumine is prepared by weighing the gadopentetate dimeglumine and thoroughly mixing the gadopentetate dimeglumine with a NASHA-gel (20 mg HA/ml, Q-Med) by manual stirring. The resulting gel is centrifuged to remove air bubbles.

The release of the gadolinium complexes from the gels is measured using a USP-paddle system. The release of the gadolinium complex is followed using NMR and ICP-MS. A 2D spin echo (SE) sequence and a 3D gradient Echo (FFE) sequence are made, both sequences are made with, and without, fat saturation (FS). MRI is also made on gels that release the gadolinium complexes for different periods of time. The initial release of the gadolinium complex should be relatively fast and the rate should correspond well with the diffusion rate of small drug molecules. The ICP-MS analysis after 7 and 24 hours release will show that about 80% of the gadolinium will be released by 1 hour after adding, indicating that a small amount of gadolinium interacts and was released at a much slower rate. In the MRI measurements on the gel, at the first time points (30 minutes and 90 minutes) the NASHA-gel shows a strong contrast compared to water and oil. At the later times (8 hours up to 4 days) the contrast is much weaker but still visible compared to NASHA-gel without the gadolinium complexes. Further, the gadolinium complexes are retained in the gels in a dose-dependent manner (more gadolinium results in more contrast on MRI) showing that a small amount of gadolinium seems to interact with the NASHA-gel. Gels containing more than 5 mg/ml, more than 6 mg/ml, more than 7 mg/ml, more than 8 mg/ml, more than 9 mg/ml, or more than 10 mg/ml gadopentetate dimeglumine give a contrast useful for in vivo MRI at the later time points.

### Example 18: Preparing and Injecting a Viscoelastic Media Gel with 45 mg of Gold Particle Additives

In one example, 3 ml of gel was prepared as a viscous mixture of 1 mg/ml hyaluronic acid (HA) and 45 mg of gold microparticles as a visual additive, the gel having gel particles of an average size of 1 mm and where the gold microparticles had a size of 80 micrometers. Following local anesthesia of subcutaneous tissue with lidocaine and under ultrasound and CT guidance, a 21-gauge needle was inserted at a puncture point lateral to the rectum and advanced to the location for gel injection. To advance the needle safely, the rectum was shifted by about 5-10 mm manually to the right or left when necessary. Using this route, the needle passes medially to the sternohyoid and stemothyroid strap muscles to avoid the influence of muscle contraction on the needle. When the needle tip reached the predetermined injection point, the gel was injected to create a space, forcing the esophagus away from the target. The created space was confirmed by CT.

The gel was at least partially inserted in fat associated with the rectum and scanned using CT and CBCT, with the scans being obtained after the injection. The contrast to noise ratio of the CT scan of the injected tissue and surrounding tissue was at least 10.6±5.4. The contrast to noise ratio of the CBCT scan of the injected tissue and surrounding tissue was at least 14.3±1.5.

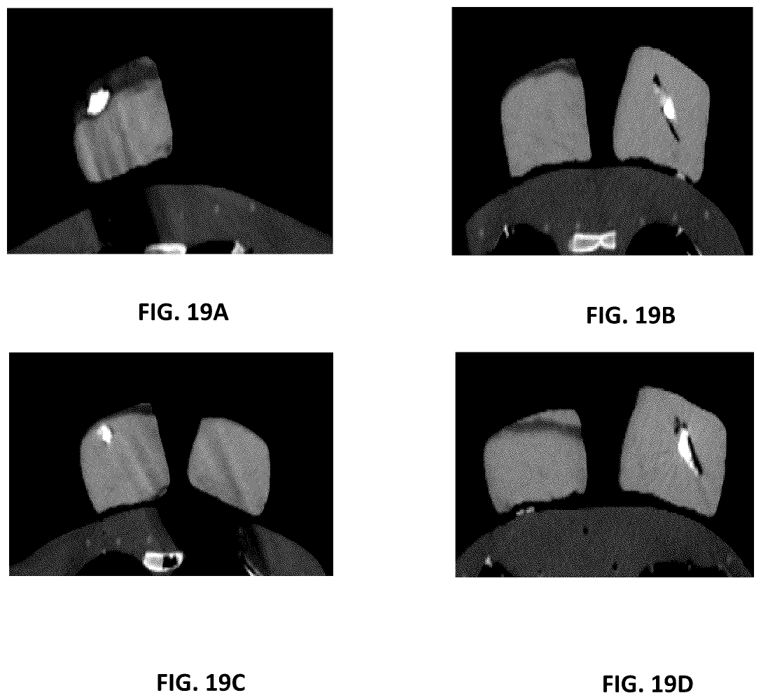

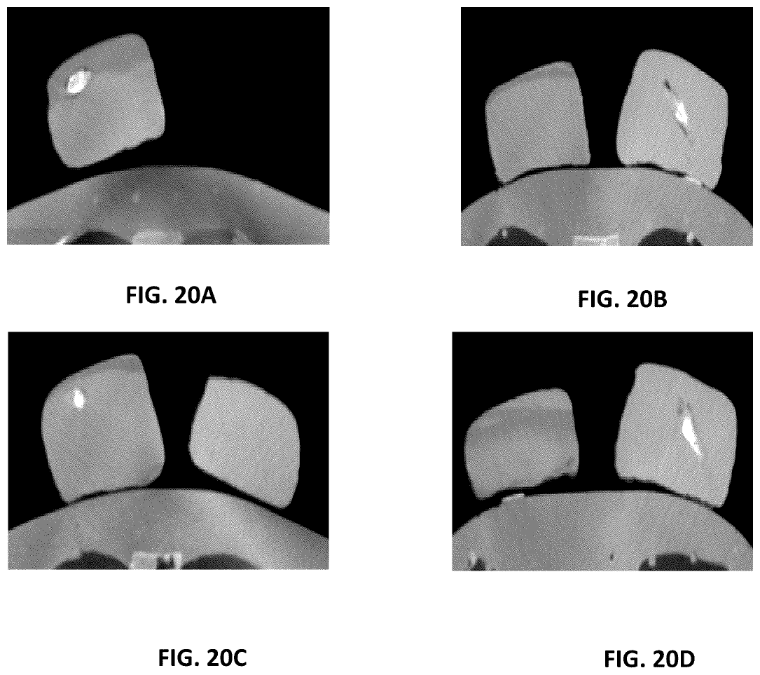

The gel was at least partially inserted in muscle associated with the rectum and scanned using CBCT, with scans (as shown in FIGS. 19B, 19C, 20B, and 20C) being obtained after the injection. The contrast to noise ratio of the CT scan (as shown in FIGS. 19B and 19C) of the injected tissue and surrounding tissue was at least 10.0±2.0, which is both adequate for practitioner use and improved relative to contrast to noise ratios of CT scans of gels not containing the visual additive. The contrast to noise ratio of the CBCT scan (as shown in FIGS. 20B and 20C) of the injected tissue and surrounding tissue was at least 20.2±4.2, which is both adequate for practitioner use and improved relative to contrast to noise ratios of CBCT scans of gels not containing the visual additive.

The gel was also inserted in polyacrylamide and scanned using CT and CBCT, with scans being obtained after the injection. The contrast to noise ratio of the CT scan of the injected tissue and the surrounding tissue was 10.0±2.0, which is both adequate for practitioner use and improved relative to contrast to noise ratios of CT scans of gels not containing the visual additive. The contrast to noise ratio of the CBCT scan of the injected tissue and the surrounding tissue was 15.9±3.2, which is both adequate for practitioner use and improved relative to contrast to noise ratios of CBCT scans of gels not containing the visual additive.

### Example 19: Preparing and Injecting a Viscoelastic Media Gel with 90 mg of Gold Particle Additives

In one example, 3 ml of gel, was prepared as a viscous mixture of 1 mg/ml hyaluronic acid (HA) and 90 mg of gold microparticles as a visual additive, where the gel had gel particles with an average size of 2 mm and where the gold particles had an average size of 120 micrometers. Following local anesthesia of subcutaneous tissue with lidocaine and under ultrasound and CT guidance, a 21-gauge needle was inserted at a puncture point lateral to the rectum and advanced to the location for gel injection. To advance the needle safely, the rectum was shifted by about 5-10 mm manually to the right or left when necessary. Using this route, the needle passes medial to the stemohyoid and sternothyroid strap muscles to avoid the influence of muscle contraction on the needle. When the needle tip reached the predetermined injection point, the gel was injected to create a space, forcing the esophagus away from the target. The created space was confirmed by CT.

The gel was at least partially inserted in fat associated with the rectum and scanned using CT and CBCT, with the scans (as shown in FIGS. 19A and 20A) being obtained after the injection. The contrast to noise ratio of the CT scan (as shown in FIG. 19A) of the injected tissue and surrounding tissue was at least 10.6±5.4, which is both adequate for practitioner use and improved relative to contrast to noise ratios of CT scans of gels not containing the visual additive. The contrast to noise ratio of the CBCT scan (as shown in FIG. 20A) of the injected tissue and surrounding tissue was at least 14.3±1.5, which is both adequate for practitioner use and improved relative to contrast to noise ratios of CBCT scans of gels not containing the visual additive.

The gel was at least partially inserted in muscle associated with the rectum and scanned using CBCT, with scans (as shown in FIGS. 19D and 20D) being obtained after the injection. The contrast to noise ratio of the CT scan (as shown in FIG. 19D) of the injected tissue and surrounding tissue was at least 10.0±2.0, which is both adequate for practitioner use and improved relative to contrast to noise ratios of CT scans of gels not containing the visual additive. The contrast to noise ratio of the CBCT scan (as shown in FIG. 20D) of the injected tissue and surrounding tissue was at least 20.2±4.2, which is both adequate for practitioner use and improved relative to contrast to noise ratios of CBCT scans of gels not containing the visual additive.

The gel was also inserted in polyacrylamide and scanned using CT and CBCT, with scans being obtained after the injection. The contrast to noise ratio of the CT scan of the injected tissue and the surrounding tissue was 10.0±2.0, which is both adequate for practitioner use and improved relative to contrast to noise ratios of CT scans of gels not containing the visual additive. The contrast to noise ratio of the CBCT scan of the injected tissue and the surrounding tissue was 15.9±3.2, which is both adequate for practitioner use and improved relative to contrast to noise ratios of CBCT scans of gels not containing the visual additive.

### Example 20: Dispersion of Viscoelastic Gel within Tissue Over a Period of One Year

In one example, viscoelastic gel with gold particles is injected into human tissue, and the viscoelastic gel with gold particles' dispersion was measured over a period of one year. In this example, three different amounts are injected into three different specimens of human tissue: a first amount of 16.51 cubic centimeters (cc), a second amount of 9.34 cc, and a third amount 6.90 cc.

As shown in scan 2102a, the first amount 2104 of viscoelastic media gel with gold particles was first injected into the first specimen and scanned immediately after injection. As shown in scan 2102b, the first amount 2104 of viscoelastic media gel with gold particles was scanned again in the tissue after a period of 3 months after injection. As shown in scan 2102c, the first amount 2104 of viscoelastic media gel with gold particles was scanned again in the tissue after a period of 1 year.

As shown in scan 2112a, the second amount 2114 of viscoelastic media gel with gold particles was first injected into the second specimen and scanned immediately after injection. As shown in scan 2112b, the second amount 2114 of viscoelastic media gel with gold particles was scanned again in the tissue after a period of 3 months after injection. As shown in scan 2112c, the second amount 2114 of viscoelastic media gel with gold particles was scanned again in the tissue after a period of 1 year.

As shown in scan 2122a, the third amount 2124 of viscoelastic media gel with gold particles was first injected into the third specimen and scanned immediately after injection. As shown in scan 2122b, the third amount 2124 of viscoelastic media gel with gold particles was scanned again in the tissue after a period of 3 months after injection. As shown in scan 2122c, the third amount 2124 of viscoelastic media gel with gold particles was scanned again in the tissue after a period of 1 year.

Thus, as shown in scans 2102a-2102b, 2112a-2112b, and 2122a-2122b, the placement of the first amount 2104, second amount 2114, and third amount 2124, respectively, of viscoelastic media gel with gold particles did not substantially migrate within or resorb into the tissue from immediately after injection to 3 months after injection. However, as shown in scans 2102b-2102c, 2112b-2112c, and 2122b-2122c, the first amount 2104, second amount 2114, and third amount 2124 was almost entirely resorbed into the tissue between 3 months after injection and 1 year after injection.

### Example 21: Erasable Hyaluronic Acid Spacer

The techniques described in Examples 4 and 5 are utilized. A patient is in rapid need of a reduction in the size of the spacer following radiotherapy due to the pressure exerted by the spacer onto the patient's esophagus. Upon routine review of patient post-implant images, it is determined that the implant must be partially reduced before proceeding to radiation therapy in order to improve the probability of optimal therapeutic outcome. Hyaluronidase is administered in approximately an amount that is two times larger than the volume of the HA that is to be removed. The undesired portion of the implant then dissolves over the next week.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A method of visualizing a tissue, space, or location of a radiographic subject, comprising disposing an imaging contrast composition, said composition comprising:
a viscoelastic medium; and
a first visual additive, wherein said first visual additive is a metal having a particle diameter greater than about 80 micrometers.

2. The method of claim 1, further comprising:
(a) disposing the composition within a first tissue site, and
(b) imaging said composition within said first tissue site.

3. The method of claim 1, wherein said metal has a particle diameter of greater than 100 micrometers.

4. The method of claim 1, wherein a concentration of said first visual additive in said viscoelastic medium results in a contrast to noise ratio of about 0.1 to about 40 when imaged.

5. The method of claim 1, wherein said metal is a precious metal.

6. The method of claim 5, wherein said precious metal is chosen from the group consisting of gold (Au), iodine (I), gadolinium (Gd), iron (Fe), barium (Ba), calcium (Ca), magnesium (Mg), and combinations thereof.

7. The method of claim 1, wherein said first visual additive further comprises one or more microbubbles.

8. The method of claim 1, wherein said composition comprises a second visual additive different than said first visual additive.

9. The method of claim 8, wherein said second visual additive comprises one or more microbubbles.

10. The method of claim 1, wherein a concentration of said first visual additive in said viscoelastic medium is less than 90 mg/ml.

11. The method of claim 10, wherein said concentration of said first visual additive in said viscoelastic medium is between 15 mg/ml and 30 mg/ml.

12. The method of claim 1, wherein said composition is configured to not substantially migrate prior to or during imaging, wherein the imaging occurs about 3 months to about 9 months after the composition is disposed.

13. The method of claim 1, wherein said composition is configured to be disposed through injection.

14. The method of claim 1, wherein said viscoelastic medium comprises hyaluronic acid, polyethylene glycol, or dextranomers.

15. The method of claim 14, wherein a concentration of said hyaluronic acid, polyethylene glycol, or dextranomers in said viscoelastic medium is between about 5 mg/ml to about 100 mg/ml.

16. The method of claim 1, wherein said viscoelastic medium comprises gel particles, wherein said gel particles comprise said metal particles.

17. The method of claim 1, wherein said viscoelastic medium comprises non-animal stabilized hyaluronic acid (“NASHA”).

18. The method of claim 1, wherein said viscoelastic medium is injected one time every six months over a period of at least 12 months.

19. The method of claim 1, wherein said viscoelastic medium is completely resorbed within 20 months.

20. The method of claim 1, wherein said composition is configured to be detectable on an imaging modality for at least 9 months.
